(19) United States
(12) Patent Application Publication
(10) Pub. No.: US 2023/0245721 A1
(43) Pub. Date: Aug. 3, 2023

(54) GENERATION OF OPTIMIZED NUCLEOTIDE SEQUENCES

(71) Applicant: TRANSLATE BIO, INC., Waltham, MA (US)

(72) Inventors: Khang Anh TRAN, Cambridge, MA (US); Anusha DIAS, Cambridge, MA (US); Frank DEROSA, Cambridge, MA (US)

(21) Appl. No.: 17/923,844

(22) PCT Filed: May 7, 2021
(86) PCT No.: PCT/US2021/031302

§ 371 Date: Nov. 7, 2022

Related U.S. Application Data
(60) Provisional application No. 63/021,345, filed on May 7, 2020.

(51) Int. Cl. G16B 30/20; G16B 25/10; G16B 45/00

(52) U.S. Cl. CPC G16B 30/20; G16B 25/10; G16B 45/00

(57) ABSTRACT

A method for generated an optimized nucleotide sequence is provided. The method comprises at least normalizing a codon usage table and selection of codons for a given amino acid sequence based on the usage frequency of the codons in the normalized codon usage table. The method may comprise generating a list of a plurality of optimized nucleotide sequences encoding the amino acid sequence, filtering the list of optimized nucleotide sequences, synthesizing one or more optimized nucleotide sequence, and/or administering one or more synthesized optimized nucleotide sequence.

### RELATED APPLICATION

This application claims priority to U.S. Provisional Application Serial No. 63/021,345, filed May 7, 2020, the disclosure of which is hereby incorporated by reference in its entirety. U.S. Provisional Application Serial No. 62/978,180, filed Feb. 18, 2020, is incorporated herein by reference in its entirety.

### SEQUENCE LISTING

The present specification makes reference to a Sequence Listing (submitted electronically as a .txt file named MRT-2131WO_SL on May 7, 2021). The .txt file was generated on Apr. 27, 2021 and is 63.5 KB in size. The entire contents of the sequence listing are herein incorporated by reference.

### FIELD OF THE INVENTION

The present invention relates to methods for generating an optimized nucleotide sequence. In particular, the present invention relates to methods wherein a nucleotide sequence is optimized for in vitro synthesis and for expression of a functional protein, polypeptide or peptide encoded by the optimized nucleotide sequence in a cell.

### BACKGROUND OF THE INVENTION

mRNA therapy is increasingly important for treating various diseases, especially those caused by dysfunction of proteins or genes. Genetic mutations in the DNA sequence of an organism can lead to aberrant gene expression, resulting in defects in protein production or function. For example, mutations in an underlying DNA sequence can lead to insufficient expression or over-expression of a protein, or production of dysfunctional proteins. Restoration of normal or healthy levels of the protein can be achieved through mRNA therapy, which is widely applicable to a range of diseases caused by gene or protein dysfunction.

In mRNA therapy, mRNA encoding a functional protein that can replace a defective or missing protein is delivered to a target cell or tissue. Administration of an mRNA encoding a therapeutic protein efficacious in treating or preventing a disease or disorder can also provide a cost-effective alternative to therapy with a recombinantly produced peptide, polypeptide or protein. mRNA therapy can restore the normal levels of an endogenous protein or provide an exogenous therapeutic protein without permanently altering the genome sequence or entering the nucleus of the cell. mRNA therapy takes advantage of the cell’s own protein production and processing machinery to treat diseases or disorders, is flexible to tailored dosing and formulation, and is broadly applicable to any disease or condition caused by an underlying gene or protein defect or treatable through the provision of an exogenous protein.

Expression levels of an mRNA-encoded protein can significantly impact the efficacy and therapeutic benefits of mRNA therapy. Effective expression or production of a protein from an mRNA within a cell depends on a variety of factors. Optimization of the composition and order of codons within a protein-coding nucleotide sequence (“codon optimization”) can lead to higher expression of the mRNA-encoded protein. Various methods of performing codon optimization are known in the art, however, each has significant drawbacks and limitations from a computational and/or therapeutic point of view. In particular, known methods of codon optimization often involve, for each amino acid, replacing every codon with the codon having the highest usage for that amino acid, such that the “optimized” sequence contains only one codon encoding each amino acid (so may be referred to as a one-to-one sequence).

Accordingly, a need exists for improved codon optimization methods that generate an optimized nucleotide sequence for increased expression of protein in mRNA therapy.

### SUMMARY OF THE INVENTION

The present invention addresses the need for improved nucleic acid optimization methods for effective mRNA therapy by providing a method for analyzing an amino acid sequence to produce at least one optimized nucleotide sequence. The optimized nucleotide sequence is designed to increase the expression of a protein compared to the expression of the protein associated with a naturally occurring nucleotide sequence. The nucleic acid optimization methods of the invention provide the ability to synthesize full-length mRNA transcripts in vitro and increase the expression of a protein of interest in settings where it is desirable to achieve higher protein yield.

For example, codon optimization can be used to increase expression of a protein of interest in mRNA therapy, immunology and vaccination, cancer immunotherapy, biotechnology, and manufacturing. Codon optimization produces a protein-coding nucleotide sequence based on various criteria without altering the sequence of translated amino acids of the encoded protein, due to the redundancy in the genetic code.

To avoid imbalance between mRNA codon usage and abundance of cognate tRNAs, codon optimization can provide a composition of codons within a nucleotide sequence that better matches the naturally occurring abundance of transfer RNAs (tRNAs) in a host cell and avoid depletion of a specific tRNA. As tRNA abundance influences the rate of protein translation, codon optimization of a nucleotide sequence can increase the efficiency of protein translation and yield for the encoded protein. For example, by not using rare codons, which are characterized by a low codon usage, efficiency of protein translation and protein yield can be increased, as the shortage of rare tRNAs can stall or terminate protein translation. However, codon optimization can come at the cost of reduced functional activity of the encoded protein and an associated loss in efficacy as the process may remove information encoded in the nucleotide sequence that is important for controlling translation of the protein and ensuring proper folding of the nascent polypeptide chain (Mauro & Chappell, Trends Mol Med. 2014; 20(11):604-13). The inventors have found that optimized sequences which retain some variety, i.e. do not necessarily include only one codon encoding each amino acid, can achieve increased protein yield over both naturally occurring sequences and one-to-one sequences.

In a first aspect, the present invention relates to a computer-implemented method for generating an optimized nucleotide sequence, comprising: (i) receiving an amino acid sequence, wherein the amino acid sequence encodes a peptide, polypeptide, or protein; (ii) receiving a first codon usage table, wherein the first codon usage table comprises a list of amino acids, wherein each amino acid in the table is associated with at least one codon and each codon is associated with a usage frequency; (iii) removing from the codon usage table any codons associated with a usage frequency which is less than a threshold frequency; (iv) generating a normalized codon usage table by normalizing the usage frequencies of the codons not removed in step (iii); and (v) generating an optimized nucleotide sequence encoding the amino acid sequence by selecting a codon for each amino acid in the amino acid sequence based on the usage frequency of the one or more codons associated with the amino acid in the normalized codon usage table. In some embodiments, the threshold frequency is selectable by a user. In some embodiments, the threshold frequency is in the range of 5% - 30%, in particular 5%, or 15%, or 20%, or 25%, or 30%, or, in particular, 10%. The inventors have found that threshold frequencies having values as described herein may generate optimized sequences that may achieve increased protein yield.

In some embodiments, the step of generating a normalized codon usage table comprises: (a) distributing the usage frequency of each codon associated with a first amino acid and removed in step (iii) to the remaining codons associated with the first amino acid; and (b) repeating step (a) for each amino acid to produce a normalized codon usage table. In some embodiments, the usage frequency of the removed codons is distributed equally amongst the remaining codons. In some embodiments, the usage frequency of the removed codons is distributed amongst the remaining codons proportionally based on the usage frequency of each remaining codon.

In some embodiments, selecting a codon for each amino acid comprises: (a) identifying, in the normalized codon usage table, the one or more codons associated with a first amino acid of the amino acid sequence; (b) selecting a codon associated with the first amino acid, wherein the probability of selecting a certain codon is equal to the usage frequency associated with the codon associated with the first amino acid in the normalized codon usage table; and (c) repeating steps (a) and (b) until a codon has been selected for each amino acid in the amino acid sequence.

In some embodiments, the step of generating an optimized nucleotide sequence by selecting a codon for each amino acid in the amino acid sequence (step (v) in the above method) is performed n times to generate a list of optimized nucleotide sequences.

In some embodiments, the method further comprises: screening the list of optimized nucleotide sequences to identify and remove optimized nucleotide sequences failing to meet one or more criteria. In this way, the method allows a significant number of candidate optimized nucleotide sequences to be removed from consideration if the chance that they are effective is reduced by failing to meet one or more criteria. In other words, the criteria are indicative of practical effectiveness of the optimized nucleotide sequence, so nucleotide sequences failing to meet one or more criteria can be excluded from further consideration. The one or more criteria may comprise: the sequence not containing one or more termination signals; the sequence having a guanine-cytosine content falling within a predetermined range; the sequence having a codon adaptation index greater than a threshold value; the sequence not containing one or more CIS elements; the sequence not containing one or more repeat elements; and other criteria of interest.

In this way, the method provides a shorter, or filtered, list of optimized nucleotide sequences. By reducing the number of optimized nucleotide sequences in the list, further steps performed on the sequences in the list, for example further algorithmic steps or physical synthesis steps, are advantageously reduced in number and complexity.

In some embodiments, screening the list of optimized nucleotide sequences comprises, for a certain criterion: determining whether each optimized nucleotide sequence in the list, or most recently updated list, of optimized nucleotide sequences meets the criterion; and updating the list of optimized nucleotide sequences by removing any nucleotide sequence from the list, or most recently updated list, if the nucleotide sequence does not meet the criterion.

In some embodiments, determining whether each optimized nucleotide sequence in the list, or most recently updated list, of optimized nucleotide sequences meets the criterion comprises, for each nucleotide sequence: determining whether a first portion of the nucleotide sequence meets the criterion, and wherein updating the list of optimized nucleotide sequences comprises: removing the nucleotide sequence if the first portion does not meet the criterion. In some embodiments, determining whether each optimized nucleotide sequence in the list, or most recently updated list, of optimized nucleotide sequences meets the criterion further comprises, for each nucleotide sequence: determining whether one or more additional portions of the nucleotide sequence meets the criterion, wherein the additional portions are non-overlapping with each other and with the first portion, and updating the list of optimized sequences comprises: removing the nucleotide sequence if any portion does not meet the criterion, optionally wherein determining whether an optimized nucleotide sequence meets the criterion is halted when any portion is determined not to meet the criterion.

By filtering the optimized nucleotide sequences in this way, the method is computationally advantageous, because sequences may be discarded from the list before computing and time resources have been spent on analyzing the entire sequence. Thus, the method is advantageously more efficient. Furthermore, for some criteria, analyzing by portion provides a more detailed and selective screening process. Using guanine-cytosine content as an example, the method not only removes sequences for which an average guanine-cytosine content falls outside the predetermined range, but also advantageously removes any sequence having a spike or trough of guanine-cytosine content in a particular portion which could hinder efficient transcription or translation. Such peaks or troughs could be missed if the entire sequence were only analyzed all at once, because the portions of the sequence outside the analyzed portion could bring the average guanine-cytosine content within the allowable range. By analyzing portion by portion, not only can computationally efficiency be improved, but issues in candidate sequences which are otherwise masked in the average can be identified.

Although guanine-cytosine content has been used as an example here, it will be appreciated that any criterion described herein may be analyzed portion by portion as above. For some criteria, for example the sequence containing a termination signal, computational efficiency will be increased, but the outcome of screening by portion will not have an effect on the contents of the resulting list, i.e. assessing termination signals in portions will remove the same nucleotide sequences from the list as would assessing the entire sequence. For others, for example guanine-cytosine content or codon adaptation index, the outcome of the screening may be different, for example certain sequences may be removed using a potion analysis that would not have been removed when assessing sequences in their entirety.

The first portion and/or the one or more additional portions of the nucleotide sequence may comprise a predetermined number of nucleotides, optionally the predetermined number of nucleotides is in the range of: 5 to 300 nucleotides, or 10 to 200 nucleotides, or 15 to 100 nucleotides, or 20 to 50 nucleotides, e.g., 30 nucleotides, e.g., 100 nucleotides. It has been found that portions of this length provide an optimal balance between

In some embodiments, a first criterion comprises the nucleotide sequence not containing a termination signal, such that the method comprises: determining whether each optimized nucleotide sequence in the list, or most recently updated list, of optimized nucleotide sequences contains a termination signal; and updating the list of optimized nucleotide sequences by removing any nucleotide sequence from the list, or most recently updated list, if the nucleotide sequence contains one or more termination signals.

In this way, the method provides a shorter, or filtered, list of optimized nucleotide sequences. By reducing the number of optimized nucleotide sequences in the list, further steps performed on the sequences in the list, for example further algorithmic steps or physical synthesis steps, are advantageously reduced in number and complexity. In some embodiments, the termination signal has the following nucleotide sequence: 5′-X₁ATCTX₂TX₃-3′, wherein X₁, X₂ and X₃ are independently selected from A, C, T or G. In some embodiments, the termination signal has one of the following nucleotide sequences: TATCTGTT; and/or TTTTTT; and/or AAGCTT; and/or GAAGAGC; and/or TCTAGA. In some embodiments, the termination signal has the following nucleotide sequence: 5′-X₁AUCUX₂UX₃-3′, wherein X₁, X₂ and X₃ are independently selected from A, C, U or G. In some embodiments, the termination signal has one of the following nucleotide sequences: UAUCUGUU; and/or UUUUUU; and/or AAGCUU; and/or GAAGAGC; and/or UCUAGA.

In some embodiments, a second criterion comprises the nucleotide sequence having a guanine-cytosine content within a predetermined guanine-cytosine content range, such that the method comprises: determining a guanine-cytosine content of each of the optimized nucleotide sequences in the list, or most recently updated list, of optimized nucleotide sequences, wherein the guanine-cytosine content of a sequence is the percentage of bases in the nucleotide sequence that are guanine or cytosine; updating the list of optimized nucleotide sequences by removing any nucleotide sequence from the list, or most recently updated list, if its guanine-cytosine content falls outside a predetermined guanine-cytosine content range. By reducing the number of optimized nucleotide sequences in the list, further steps performed on the sequences in the list, for example further algorithmic steps or physical synthesis steps, are advantageously reduced in number and complexity. In some embodiments, the predetermined guanine-cytosine content range is 15% - 75%, or 40% - 60%, or, in particular, 30% - 70%.

In some embodiments, a third criterion comprises the nucleotide sequence having a codon adaptation index greater than a predetermined codon adaptation index threshold, such that the method comprises: determining a codon adaptation index of each of the optimized nucleotide sequences in the list, or most recently updated list, of optimized nucleotide sequences, wherein the codon adaptation index of a sequence is a measure of codon usage bias and can be a value between 0 and 1; updating the list, or most recently updated list, of optimized nucleotide sequences by removing any nucleotide sequence if its codon adaptation index is less than or equal to a predetermined codon adaptation index threshold. In this way, the method provides a shorter, or filtered, list of optimized nucleotide sequences. In some embodiments, the codon adaptation index threshold is selectable by a user. In some embodiments, the codon adaptation index threshold is 0.7, or 0.75, or 0.85, or 0.9, or, in particular, 0.8. By reducing the number of optimized nucleotide sequences in the list, further steps performed on the sequences in the list, for example further algorithmic steps or physical synthesis steps, are advantageously reduced in number and complexity.

In some embodiments, a fourth criterion comprises the nucleotide sequence not containing at least 2, for example 3, adjacent identical codons, such that the method further comprises: determining whether any optimized nucleotide sequence in the list, or most recently updated list, of optimized nucleotide sequences, containing at least 2, for example 3, adjacent identical codons; and updating the list, or most recently updated list, of optimized nucleotide sequences by removing any nucleotide sequence if it contains at least 2, for example 3, adjacent identical codons. It has been found that repeated identical codons, in other words adjacent identical codons, can stall transcription. Therefore, by removing from the list any optimized nucleotide sequence containing 2 or more, 4 or more, 5 or more, 6 or more, 7 or more, 8 or more, 9 or more, or, in particular, 3 or more, identical adjacent codons, sequences providing less effective transcription can be disregarded and removed.

In any aspect of the invention, the generation of an updated list of optimized nucleotide sequences may be performed by removing optimized sequences from the list based on any one of, any two of, or any three of the following steps:
(I) determining a determining the presence of a termination signal in one or more optimized nucleotide sequences and removing nucleotide sequences from the list, or most recently updated list, of optimized nucleotide sequences if they contain the termination signal;
(II) determining a guanine-cytosine content of one or more optimized nucleotide sequences and removing nucleotide sequences from the list, or most recently updated list, of optimized nucleotide sequences if their guanine-cytosine content falls outside a predetermined range;
(III) determining a codon adaptation index of one or more optimized nucleotide sequences and removing nucleotide sequences from the list, or most recently updated list, of optimized nucleotide sequences if their guanine-cytosine content falls outside a predetermined range.

In a second aspect of the invention, after the generation of one or more optimized nucleotide sequences, the method further comprises performing step (I).

In a third aspect of the invention, after the generation of one or more optimized nucleotide sequences, the method further comprises performing step (II).

In a fourth aspect of the invention, after the generation of one or more optimized nucleotide sequences, the method further comprises performing step (III).

In a fifth aspect of the invention, after the generation of one or more optimized nucleotide sequences, the method further comprises performing step (I), then step (II).

In a sixth aspect of the invention, after the generation of one or more optimized nucleotide sequences, the method further comprises performing step (I), then step (III).

In a seventh aspect of the invention, after the generation of one or more optimized nucleotide sequences, the method further comprises performing step (II), then step (I).

In an eighth aspect of the invention, after the generation of one or more optimized nucleotide sequences, the method further comprises performing step (II), then step (III).

More typically, a method in accordance with the invention comprises termination signal based steps (I), guanine-cytosine content based steps (II), and codon adaptation index based steps (III) in order to produce a shortlist of optimized nucleotide sequences that are all expected to provide a full-length mRNA transcript when synthesized by in vitro transcription and to yield high levels of expression of the mRNA-encoded protein in vivo. The termination signal based steps (I), guanine-cytosine content based steps (II), and codon adaptation index based steps (III) may be performed in any order. Advantageously, the steps may be performed in a specific order for the purpose of optimizing computation time when determining the shortlist of optimized nucleotide sequences.

In a ninth, particular, aspect of the invention, after the generation of one or more optimized nucleotide sequences, the method further comprises performing step (I), then step (II), then step (III). By filtering in this order, the computational efficiency of the filtering steps may be advantageously maximized. The inventors have found that, for a typical list of optimized nucleotide sequences and typical input parameters, the motif screen filter removes the most sequences from the list, followed by the GC content analysis filter, followed by the CAI analysis filter. Since the computational efficiency of the filtering process is in part determined by the total number of sequences analyzed, i.e. the sum of the sequences analyzed in each filtering step, the more sequences can be removed early in the filtering process, the fewer sequences require analysis later in the filtering process, thus increasing the overall computational efficiency of the method. Furthermore, the CAI analysis filter requires analysis of the whole sequence, whereas in embodiments of the invention the motif screen and GC content analysis filters may only analyze parts, or portions, of a sequence. Thus, a method which emphasizes reducing the number of sequences in the list input to the CAI analysis step will likely be more computationally efficient than other methods.

In a tenth aspect of the invention, after the generation of one or more optimized nucleotide sequences, the method further comprises performing step (I), then step (III), then step (II).

In an eleventh aspect of the invention, after the generation of one or more optimized nucleotide sequences, the method further comprises performing step (II), then step (I), then step (III).

In a twelfth aspect of the invention, after the generation of one or more optimized nucleotide sequences, the method further comprises performing step (II), then step (III), then step (I).

In a thirteenth aspect of the invention, after the generation of one or more optimized nucleotide sequences, the method further comprises performing step (III), then step (I), then step (II).

In a fourteenth aspect of the invention, after the generation of one or more optimized nucleotide sequences, the method further comprises performing step (III), then step (II), then step (I).

In some embodiments, the amino acid sequence is received from a database of amino acid sequences. In some embodiments, the method further comprises requesting the amino acid sequence from the database of amino acid sequences, wherein the amino acid sequence is received in response to the request.

In some embodiments, the first codon usage table is received from a database of codon usage tables. In some embodiments, the method further comprises requesting the first codon usage table from the database of codon usage tables, wherein the first codon usage table is received in response to the request.

In a fifteenth aspect, the present invention relates to a computer program comprising instructions which, when the program is executed by a computer, cause the computer to carry out a method according to any embodiment of the first aspect.

In a sixteenth aspect, the present invention relates to a data processing system comprising means for carrying out a method according to any embodiment of the first aspect.

In a seventeenth aspect, the present invention relates to a computer-readable data carrier having stored thereon the computer program of the third aspect.

In a eighteenth aspect, the present invention relates to a data carrier signal carrying the computer program of the third aspect.

In a nineteenth aspect, the present invention relates to a method for synthesizing a nucleotide sequence, comprising: performing a method according to any embodiment of the first aspect to generate at least one optimized nucleotide sequence; and synthesizing at least one of the generated optimized nucleotide sequences. In some embodiments, the method further comprises inserting at least one of the synthesized optimized sequences in a nucleic acid vector for use in vitro transcription.

In some embodiments, the method further comprises inserting one or more termination signals at the 3′ end of the synthesized optimized nucleotide sequences. In some embodiments, more than one termination signal is inserted, and said termination signals are separated by 10 base pairs or fewer, e.g. separated by 5-10 base pairs. In some embodiments, the one or more termination signals have the following nucleotide sequence: 5′-X₁ATCTX₂TX₃-3′, wherein X₁, X₂ and X₃ are independently selected from A, C, T or G. In some embodiments, the one or more termination signals have one of the following nucleotide sequences: TATCTGTT; TTTTTT; AAGCTT; GAAGAGC; and/or TCTAGA. In some embodiments, the more than one termination signals are encoded by the following nucleotide sequence: (a) 5′-X₁ATCTX₂TX₃-(Z_{N})- X₄ATCTX₅TX₆-3′ or (b) 5′-X₁ATCTX₂TX₃-(Z_{N})- X₄ATCTX₅TX₆-(Z_{M})- X₇ATCTX₈TX₉-3′, wherein X₁, X₂, X₃, X₄, X₅, X₆, X₇, X₈ and X₉ are independently selected from A, C, T or G, Z_{N} represents a spacer sequence of N nucleotides, and Z_{M} represents a spacer sequence of M nucleotides, each of which are independently selected from A, C, T of G, and wherein N and/or M are independently 10 or fewer.

In some embodiments, the nucleic acid vector comprises an RNA polymerase promoter operably linked to the optimized nucleotide sequence, optionally wherein the RNA polymerase promoter is a SP6 RNA polymerase promoter or a T7 RNA polymerase promoter. In some embodiments, the nucleic acid vector comprises a nucleotide sequence encoding a 5′ UTR operably linked to the optimized nucleotide sequence. In some embodiments, the 5′ UTR is different to the 5′ UTR of a naturally occurring mRNA encoding the amino acid sequence. In some embodiments, the 5′ UTR has the nucleotide sequence of SEQ ID NO: 16. In some embodiments, the nucleic acid vector comprises a nucleotide sequence encoding a 3′ UTR operably linked to the optimized nucleotide sequence. In some embodiments, the 3′ UTR is different to the 3′ UTR of a naturally occurring mRNA encoding the amino acid sequence. In some embodiments, the 3′ UTR has the nucleotide sequence of SEQ ID NO: 17 or SEQ ID NO: 18. In some embodiments, the nucleic acid vector is a plasmid. In some embodiments, the plasmid is linearized before in vitro transcription. In some embodiments, the plasmid is not linearized before in vitro transcription. In some embodiments, the plasmid is supercoiled.

In some embodiments, the method further comprises using at least one of the synthesized optimized nucleotide sequences in in vitro transcription to synthesize mRNA. In some embodiments, the mRNA is synthesized by a SP6 RNA polymerase. In some embodiments, the SP6 RNA polymerase is a naturally occurring SP6 RNA polymerase. In some embodiments, the SP6 RNA polymerase is a recombinant SP6 RNA polymerase. In some embodiments, the SP6 RNA polymerase comprises a tag. In some embodiments, the tag is a his-tag. In some embodiments, the mRNA is synthesized by a T7 RNA polymerase.

In some embodiments, the method further comprises a separate step of capping and/or tailing the synthesized mRNA. In some embodiments, capping and tailing occurs during in vitro transcription.

In some embodiments, the mRNA is synthesized in a reaction mixture comprising NTPs at a concentration ranging from 1-10 mM each NTP, the DNA template at a concentration ranging from 0.01-0.5 mg/ml, and the SP6 RNA polymerase at a concentration ranging from 0.01-0.1 mg/ml. In some embodiments, the reaction mixture comprises NTPs at a concentration of 5 mM each NTP, the DNA template at a concentration of 0.1 mg/ml, and the SP6 RNA polymerase at a concentration of 0.05 mg/ml.

In some embodiments, the mRNA is synthesized at a temperature ranging from 37-56° C.

In some embodiments, the NTPs are naturally-occurring NTPs. In some embodiments, the NTPs comprise modified NTPs.

In some embodiments, the method further comprises synthesizing a reference nucleotide sequence encoding the amino acid sequence and the at least one synthesized optimized nucleotide sequence in accordance with a method of the invention, and contacting the reference nucleotide sequence and the at least one optimized nucleotide sequence with a separate cell or organism. In a typical embodiment, the cell or organism contacted with the at least one synthesized optimized nucleotide sequence produces an increased yield of the protein encoded by the optimized nucleotide sequence compared to the yield of the protein encoded by the reference nucleotide sequence produced by the cell or organism contacted with the synthesized reference nucleotide sequence. In any aspect of the invention, at least one optimized nucleotide sequence, when synthesized, may be configured to increase the expression of a protein compared to the expression of the protein encoded by the reference nucleotide sequence, when synthesized. The reference nucleotide sequence may be: (a) a naturally occurring nucleotide sequence encoding the amino acid sequence; or (b) a nucleotide sequence encoding the amino acid sequence generated by a method other than a method according to the first aspect of the invention.

In some embodiments, the method further comprises transfecting the synthesized optimized nucleotide sequence into a cell either in vitro or in vivo. In some embodiments, the expression level of the protein encoded by the synthesized optimized nucleotide sequence in the transfected cell is determined. In some embodiments, the functional activity of the protein encoded by the synthesized optimized nucleotide sequence in the transfected cell is determined.

In a twentieth aspect, the invention provides a synthesized optimized nucleotide sequence generated according to a method of the invention for use in therapy. Included in this aspect of the invention are methods of treatment comprising administering the synthesized optimized nucleotide sequence generated according to a method of the invention to a human subject in need of such treatment. In some embodiments, the methods described herein provide a therapeutic composition comprising an mRNA encoding a therapeutic peptide, polypeptide, or protein for use in the delivery to or treatment of a subject. In some embodiments, the mRNA encodes cystic fibrosis transmembrane conductance regulator (CFTR) protein.

In a twenty-first aspect, the invention provides an in vitro synthesized nucleic acid comprising an optimized nucleotide sequence consisting of codons associated with a usage frequency which is greater than or equal to 10%; wherein the optimized nucleotide sequence:
(i) does not contain a termination signal having one of the following nucleotide sequences:
   5′-X₁AUCUX₂UX₃-3′, wherein X₁, X₂ and X₃ are independently selected from A, C, U or G; and 5′-X₁AUCUX₂UX₃-3′, wherein X₁, X₂ and X₃ are independently selected from A, C, U or G;
(ii) does not contain any negative cis-regulatory elements and negative repeat elements; and
(iii) has a codon adaptation index greater than 0.8;
wherein, when divided into non-overlapping 30 nucleotide-long portions, each portion of the optimized nucleotide sequence has a guanine cytosine content range of 30% - 70%. In some embodiments, the optimized nucleotide sequence does not contain a termination signal having one of the following sequences: TATCTGTT; TTTTTT; AAGCTT; GAAGAGC; TCTAGA; UAUCUGUU; UUUUUU; AAGCUU; GAAGAGC; UCUAGA. In some embodiments, the nucleic acid is mRNA. In some embodiments, the in vitro synthesized nucleic acid is for use in therapy

### BRIEF DESCRIPTION OF THE DRAWINGS

Embodiments of the invention will be described, by way of example, with reference to the following drawings, in which:

FIG. 1 illustrates a codon optimization method according to an embodiment of the present invention.

FIG. 2A illustrates an exemplary codon usage table for humans (Homo sapiens), generated from one or more experimentally derived codon usage frequencies. The values in the table were derived from data accessed through the Codon Usage Database, which is based on codon usage data publically available from the NCBI GenBank database (Flat File Release 160.0).

FIG. 2B illustrates a normalized codon usage table generated by normalizing the codon usage frequencies of the exemplary codon usage table of FIG. 2A.

FIG. 3 illustrates a constructed section of a codon usage table for use with an exemplary method for codon usage table normalization.

FIG. 4A illustrates the exemplary table of FIG. 3, normalized with an equal usage frequency distribution.

FIG. 4B illustrates the exemplary table of FIG. 3, normalized with a proportional usage frequency distribution.

FIG. 5 illustrates a constructed section of an amino acid sequence for use with an exemplary method for codon optimization.

FIG. 6 illustrates an example repository of nucleotide sequence motifs which includes a termination signal, suitable for use in removing nucleotide sequences containing one more termination signal.

FIG. 7 illustrates a method for applying further algorithmic steps, or filtering steps, to a list of optimized nucleotide sequences. In a particular embodiment, the list of optimized nucleotide sequences for filtering has been generated according to a method as shown in FIG. 1.

FIG. 8 illustrates an embodiment of the invention in which a guanine-cytosine (GC) content analysis filter is applied to the list of optimized nucleotide sequences. In a particular embodiment, the list of optimized nucleotide sequences for filtering has been generated according to a method as shown in FIG. 1.

FIG. 9 illustrates an embodiment of the invention in which a motif screen filter and codon adaptation index (CAI) analysis filter are applied to the list of optimized nucleotide sequences. In a particular embodiment, the list of optimized nucleotide sequences for filtering has been generated according to a method as shown in FIG. 1.

FIG. 10 illustrates a particular embodiment of the invention in which a motif screen filter, guanine-cytosine (GC) content analysis filter, and codon adaptation index (CAI) analysis filter have been applied, in that order, to the list of optimized nucleotide sequences. In a particular embodiment, the list of optimized nucleotide sequences for filtering has been generated according to a method as shown in FIG. 1.

FIG. 11 illustrates an example analysis of the guanine-cytosine (GC) content of non-optimized and optimized nucleotide sequences, wherein the guanine-cytosine (GC) content of portions of the nucleotide sequence encoding EPO is determined for adjacent non-overlapping portions 30 nucleotides in length.

FIG. 12 illustrates an example bar chart depicting the yield of protein produced from various codon optimized nucleotide sequences, determined by an ELISA assay for EPO.

FIG. 13A illustrates an example western blot used to determine the protein expression yield of the CFTR protein encoded by optimized nucleotide sequences generated according to a method of the invention in a time course experiment, after the optimized nucleotide sequences were transfected into human cells.

FIG. 13B illustrates an example line plot depicting the quantification of the western blot data depicted in FIG. 13A.

FIG. 14A illustrates an example plot of data obtained from a bioassay for testing mRNAs comprising an optimized nucleotide sequence encoding hCFTR. It depicts the short circuit current (Isc) output within an Ussing epithelial voltage clamp apparatus for each tested mRNA.

FIG. 14B illustrates an example bar plot illustrating the change in hCFTR activity as depicted in FIG. 14A, expressed as a percentage of the activity of a reference mRNA encoding hCFTR.

FIG. 15A illustrates an exemplary Western blot which demonstrates the translation and expression of codon-optimized DNAI1 mRNA in HEK293T cells. The Western blot was performed with an anti-DNAI1 antibody and an anti-Vinculin antibody (loading control).

FIG. 15B illustrates an exemplary bar graph depicting the level of DNAI1 protein expression normalized to vinculin protein (loading control), quantified from the exemplary Western blot of FIG. 15A. The DNAI1 protein expression yields are graphed as fold increase relative to a reference level achieved with an mRNA encoding a DNAL1 sequence which had not been codon-optimized.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Unless specifically stated or evident from context, as used herein, the term “about” is understood as within a range of normal tolerance in the art, for example within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflects normal fluctuations that can be appreciated by a skilled artisan.

As used herein, term “abortive transcript” or “pre-aborted transcript” or the like is any transcript that is shorter than a full-length mRNA molecule encoded by the DNA template that results from the premature release of RNA polymerase from the template DNA in a sequence-independent manner. In some embodiments, an abortive transcript may be less than 90% of the length of the full-length mRNA molecule that is transcribed from the target DNA molecule, e.g., less than 80%, 70%, 60%, 50%, 40%, 30%, 20%, 10%, 5%, 1% of the length of the full-length mRNA molecule.

As used herein, the terms “codon” and “codons” refer to a sequence of three nucleotides which together form a unit of the genetic code. Each codon corresponds to a specific amino acid or stop signal in the process of translation or protein synthesis. The genetic code is degenerate, and more than one codon can encode a specific amino acid residue. For example, codons can comprise DNA or RNA nucleotides.

As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by removing with filters less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

As used herein, “full-length mRNA” is as characterized when using a specific assay, e.g., gel electrophoresis and detection using UV and UV absorption spectroscopy with separation by capillary electrophoresis. The length of an mRNA molecule that encodes a full-length polypeptide is at least 50% of the length of a full-length mRNA molecule that is transcribed from the target DNA, e.g., at least 60%, 70%, 80%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, 99.9% of the length of a full-length mRNA molecule that is transcribed from the target DNA.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “messenger RNA (mRNA)” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. A nucleic acid sequence is presented in the 5′ to 3′ direction unless otherwise indicated.

As used herein, the term “nucleotide sequence”, in its broadest sense, refers to the order of nucleobases within a nucleic acid. In some embodiments, “nucleotide sequence” refers to the order of individual nucleobases within a gene. In some embodiments, “nucleotide sequence” refers to the order of individual nucleobases within a protein-coding gene. In some embodiments, “nucleotide sequence” refers to the order of individual nucleobases within single and/or double stranded DNA and/or cDNA. In some embodiments, “nucleotide sequence” refers to the order of individual nucleobases within RNA. In some embodiments, “nucleotide sequence” refers to the order of individual nucleobases within mRNA. In a particular embodiment, “nucleotide sequence” refers to the order of individual nucleobases within the protein-coding sequence of RNA or DNA. A nucleotide sequence is normally presented in the 5′ to 3′ direction unless otherwise indicated.

As used herein, the term “premature termination” refers to the termination of transcription before the full length of the DNA template has been transcribed. As used herein, premature termination can be caused by the presence of a nucleotide sequence motif (also referred to herein simply as “motif”), e.g., a termination signal, within the DNA template and results in mRNA transcripts that are shorter than the full length mRNA (“prematurely terminated transcripts” or “truncated mRNA transcripts”). Examples of a termination signal include the E. coli rrnB terminator t1 signal (consensus sequence: ATCTGTT) and variants thereof, as described herein.

As used herein, the term “template DNA” (or “DNA template”) relates to a DNA molecule comprising a nucleic acid sequence encoding an mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 and SP6 RNA polymerases, which is operably linked to the DNA sequence encoding a desired mRNA transcript. Furthermore the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### DETAILED DESCRIPTION OF THE INVENTION

### Functions of Codon Optimization

In the process of gene expression, the nucleotide sequence encoded in the DNA sequence is transcribed into RNA molecules, and subsequently translated into proteins comprising polypeptide chains. The sequence information specifying the precise order of amino acid residues to be incorporated into the protein product is encoded in “codons” within the DNA and/or mRNA sequence. Codons comprise a sequence of three nucleotides which together form a unit of the genetic code, and each codon corresponds to a specific amino acid or stop codon signal. The genetic code is degenerate, and more than one codon can encode a specific amino acid residue.

mRNA is typically thought of as the type of RNA that carries information from DNA to the ribosome. The existence of mRNA is usually very brief and includes processing and translation, followed by degradation. Typically, in eukaryotic organisms, mRNA processing comprises the addition of a “cap” on the N-terminal (5′) end, and a “tail” on the C-terminal (3′) end. A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. The tail is typically a polyadenylation event whereby poly A moiety is added to the 3′ end of the mRNA molecule. The presence of this “tail” serves to protect the mRNA from exonuclease degradation. Messenger RNA typically is translated by the ribosomes into a series of amino acids that make up a protein.

At various steps throughout gene expression, numerous factors can affect the level to which a specific protein is expressed, or produced. For example, the presence of certain nucleotide sequence motifs can cause premature termination of transcription as the DNA sequence is transcribed into mRNA by an RNA polymerase enzyme. The specific composition and order of the codons within the protein-coding region (“coding sequence”) of a gene can also positively or negatively affect the efficiency and yield of protein expression. For example, the presence of rare codons characterized by a low codon usage frequency can negatively affect the yield of protein expression, due to the low abundance of cognate transfer RNAs encoding a specific amino acid. In biotechnological and therapeutic applications, it is often desirable to increase or maximize a protein yield when expressing said protein from the nucleotide sequence encoding it, e.g. in therapeutic applications including mRNA therapy. Codon optimization produces protein-coding nucleotide sequences based on various criteria without altering the encoded amino acid sequence, due to the redundancy in the genetic code. In other words, because multiple codons encode a single amino acid, a large number of nucleotide sequences can encode the same amino acid sequence. Codon optimization aims at producing one or more nucleotide sequences that will achieve increased protein yield.

### Amino Acid Sequences for the Generation of Optimized Nucleotide Sequences

. Naturally-occurring nucleotide sequences may be used to provide an amino acid sequence encoding a protein, polypeptide or peptide of interest. Nucleotide sequences can be obtained by isolating a nucleic acid molecule from an organism of interest and identifying the precise order of nucleobases (e.g. guanine, thymine, uracil, adenine, and cytosine) within it. There are multiple methods known in the art suitable for obtaining naturally occurring nucleotide sequences. The nucleotide sequence of protein-coding genes can be obtained by various DNA or RNA well-known sequencing methods.

For example, the DNA from a human cell can be extracted, isolated, and subsequently fragmented. The fragmented DNA can be cloned into DNA vectors and amplified in bacterial hosts, generating “libraries” of short DNA fragments. Alternatively, the fragmented DNA can be amplified using polymerase chain reaction (PCR) and incorporated into libraries suitable for high-throughput sequencing methods. The short DNA fragments derived from the original DNA material of the source organism can be sequenced individually, and subsequently assembled into a long contiguous sequence or sequences by sequence assembly. Sequence assembly is a bioinformatic approach that aligns and merges short fragments of nucleotide sequences derived from a longer nucleotide sequence, to reconstruct the original or consensus nucleotide sequence.

Nucleotide sequences generated in this manner, i.e., sequences that are experimentally derived and are known to accurately describe naturally occurring sequences, are typically stored in publically accessible repositories, or databases. For example, nucleotide sequences that can be processed according to the method of the present invention can be obtained from the GenBank database of the National Center for Biotechnology Information (NCBI). Genbank is an open access, annotated collection of publicly available nucleotide sequences and their translated protein sequences.

### Generation of Codon Usage Tables

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome. These calculations can be performed on nucleotide sequences found, for example, in the publically accessible repositories and/or databases, and also therefore represent experimentally derived data.

A codon usage table specifies the usage frequency of each codon in a given organism. Each amino acid in the table is associated with at least one codon, and each codon is associated with a usage frequency. Codon usage tables are stored in publically available databases, such as the Codon Usage Database (Nakamura et al. (2000) Nucleic Acids Research 28(1), 292; available online at https://www.kazusa.or.jp/codon/), and the High-performance Integrated Virtual Environment-Codon Usage Tables (HIVE-CUTs) database (Athey et al., (2017), BMC Bioinformatics 18(1), 391; available online at http://hive.biochemistry.gwu.edu/review/codon).

### Codon Optimization

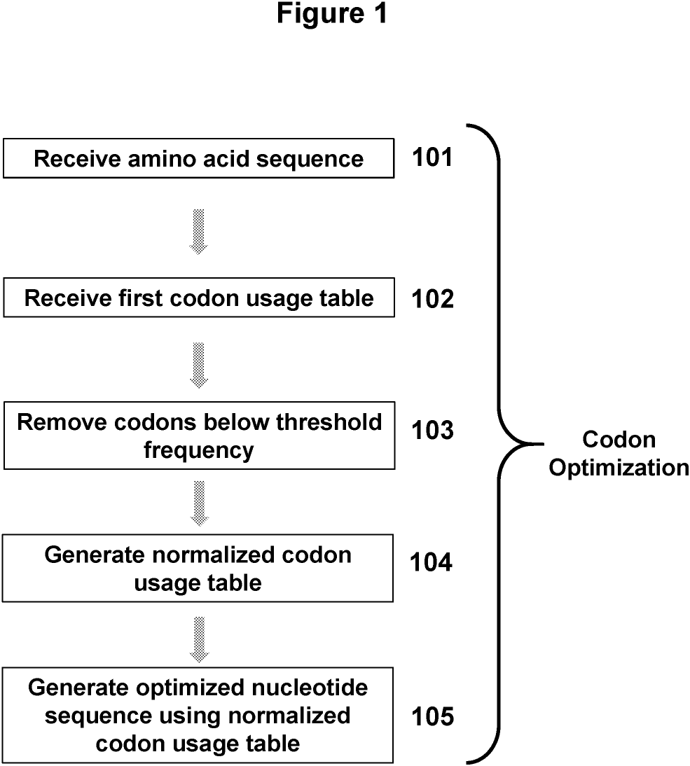

FIG. 1 illustrates a codon optimization method according to the present invention. In a first step 101, an amino acid sequence is received. The amino acid sequence may be received from a remote system, server, and/or publically accessible database and may be received wirelessly, e.g. via the internet. Alternatively, the amino acid sequence may be received from a local system, e.g., via a wired connection. The amino acid sequence comprises a plurality of amino acids.

In a second step 102, a first codon usage table is received. The first codon usage table may be received from a remote system, server and/or publically accessible database, and may be received wirelessly, e.g. via the internet. Alternatively, the first codon usage table may be received from a local system, e.g. via a wired connection. The first codon usage table comprises a list of amino acids, wherein each amino acid in the table is associated with at least one codon and each codon is associated with a usage frequency.

In a third step 103, codons are removed from the first codon usage table if they are associated with a codon usage frequency which is less than a threshold frequency.

In a fourth step 104, the codon usage frequencies of the codons not removed in the third step 103 are normalized to generate a normalized codon usage table.

In a fifth step 105, an optimized nucleotide sequence is generated by selecting a codon, for each amino acid in the amino acid sequence, based on the usage frequency of the one or more codons associated with the amino acid in the normalized codon usage table.

### Normalising the Codon Usage Table

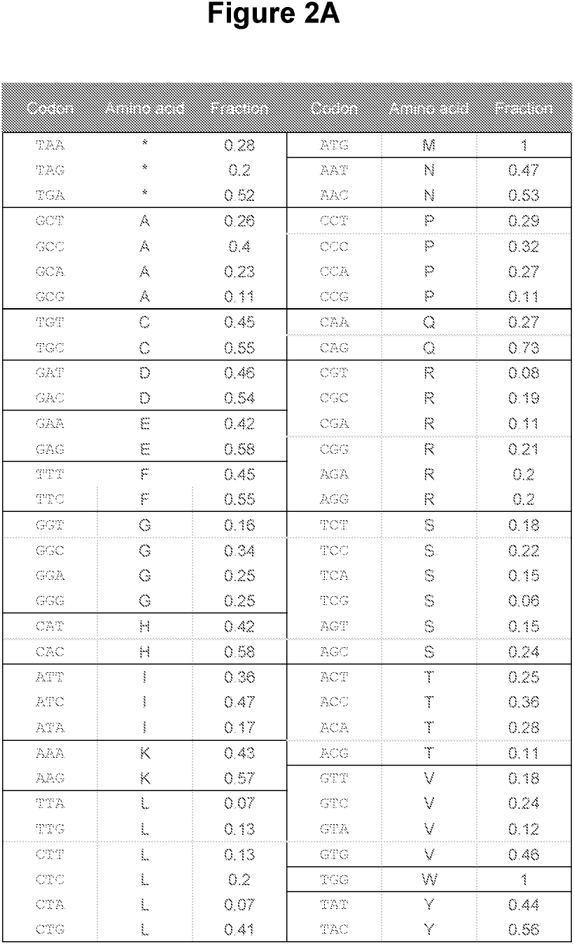

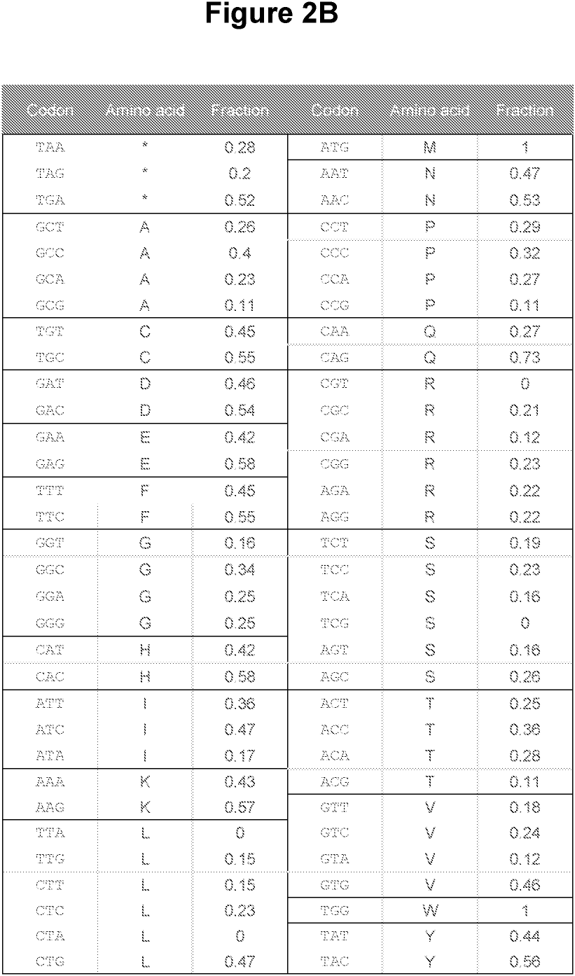

Referring to FIG. 2A, there is illustrated a codon usage table that may be found in a database of codon usage tables. The illustrated codon usage table is an example only, and it will be appreciated that any codon usage table, for example any codon usage table available on a database, may be used by the present invention to produce an optimized nucleotide sequence. The data used to produce FIG. 2A were derived from data accessed through the Codon Usage Database, based on the codon usage data publically available through the NCBI GenBank database (Flat File Release 160.0).

The codon usage table contains experimentally derived data regarding how often, for the particular biological source from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid.

FIG. 2B illustrates a normalized codon usage table that was generated from the table of FIG. 2A in accordance with a method of the invention. In the example of FIG. 2B, a threshold frequency of 10% was to perform the normalization. It will be appreciated that this is by way of example only, and that embodiments of the invention may use any other suitable threshold frequency as described herein.

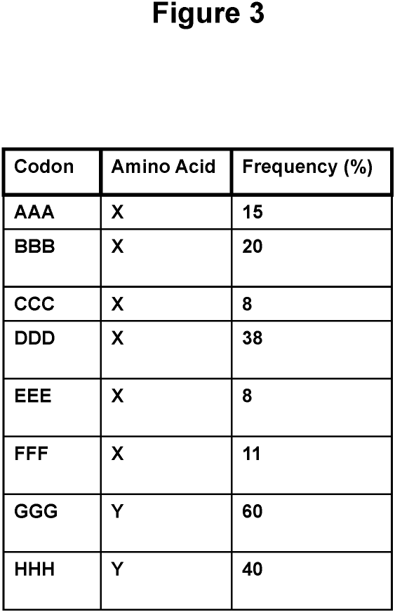

The method by which a normalized codon usage table may be provided, and was provided in the case of FIG. 2B, is illustrated in FIG. 3, which uses exemplary amino acids “X” and “Y”. It will be appreciated that, when generating the normalized codon usage table, any number of amino acids may be normalized, from one amino acid to every amino acid in the codon usage table. In the example of FIG. 3, amino acid X is encoded by codons A, B, C, D, E, and F (each codon being represented by a nucleotide triplet and thus denoted in the Figure by AAA, BBB, etc.) at the frequencies defined in the figure. Amino acid Y is encoded by codons G and H at the frequencies defined in the figure. In a first step, any codons having a usage frequency below a threshold frequency are removed from the table. It will be appreciated that, although the method illustrated in FIG. 3 uses a threshold frequency of 10%, this is by way of example only and is not intended to be limiting on the scope of the invention. The threshold frequency may be in the range of 5% - 30%, e.g., 5%, or 15%, or 20%, or 25%, or 30%, or, in particular, 10%. These values of threshold frequency have been found to provide an effective balance between increased protein yield and retaining information important for controlling translation and ensuring proper folding of the nascent polypeptide chain. It will be appreciated that the codon usage table of FIG. 3 does not accurately describe actual, naturally-occurring, codon usage, not least because it consists of only two amino acids. The table of FIG. 3 is intended to be merely illustrative of the method of codon usage table normalization.

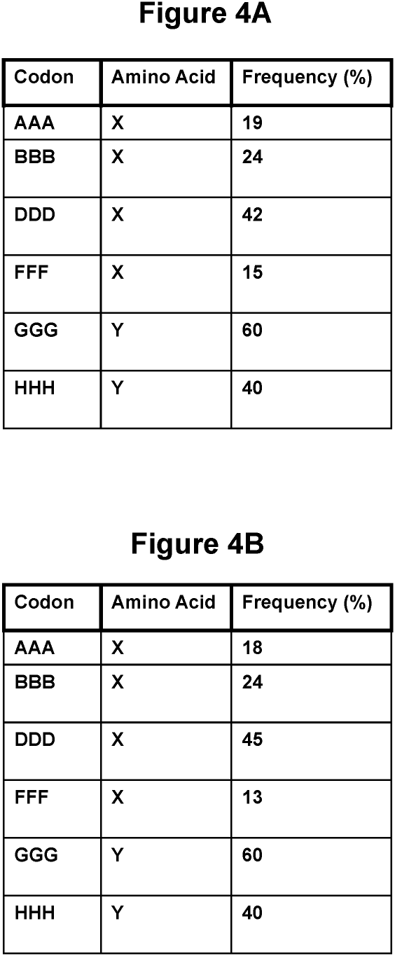

In the example of FIG. 3, codons C and E have a usage frequency below the threshold frequency of 10%, and are thus removed from the table. The combined usage frequency of the removed codons, C and E, is 16%. This combined usage frequency is then distributed amongst the remaining codons that encode for amino acid X. It is important to note that the combined usage frequency removed from amino acid X is distributed only to remaining codons that also encode for amino acid X, i.e., in the example of FIGS. 4A and 4B, the usage frequencies of codons G and H which encode amino acid Y remain unchanged.

In some embodiments, the removed combined usage frequency is distributed equally amongst the remaining codons that encode for amino acid X. Such an embodiment is illustrated in FIG. 4A. The removed combined usage frequency, 16%, has been distributed equally amongst remaining codons A, B, D, and F, so that each remaining codon has received an additional 4% usage frequency. The codon usage frequencies of amino acid X have now been normalized.

In some embodiments, the removed combined usage frequency is distributed proportionally amongst the remaining codons that encode for amino acid X. Such an embodiment is illustrated in FIG. 4B. The removed combined usage frequency, 16%, has been distributed amongst remaining codons A, B, D, and F proportional to the usage frequency of remaining codons A, B, D, and F. In this example, the usage frequency ratio of codons A, B, D, and F is 15: 20 : 38 : 11, or, 0.18 : 0.24 : 0.45 : 0.13. Codon A receives 0.18 of 16% (3%), B receives 0.24 of 16% (4%), D receives 0.45 of 16% (7%), and F receives 0.13 of 16% (2%). The codon usage frequencies of amino acid X have now been normalized.

In this way, the structure and content of the received codon usage table, or first codon usage table, instruct the generation of a normalized codon usage table. The number of codons associated with each amino acid instructs the re-distribution of removed codon usage frequencies, and the codon usage frequencies themselves instruct which codons are removed and, in some embodiments, the proportionality of the distribution.

### Generating an Optimized Nucleotide Sequence

An optimized nucleotide sequence is generated by selecting a codon, for each amino acid in the amino acid sequence, based on the usage frequency of the one or more codons associated with the amino acid in the normalized codon usage table. The optimized nucleotide sequence is generated by arranging the selected codons in the order in which their associated amino acid appears in the amino acid sequence.

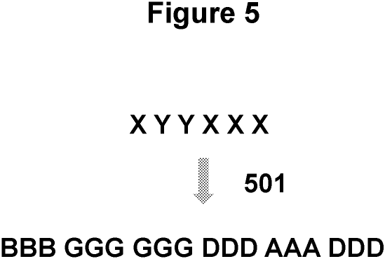

Referring to FIG. 5, there is an illustration of the generation of an optimized nucleotide sequence, using codons A, B, C, D, E, and F from FIGS. 3, 4A, and 4B. Each codon may be represented by three nucleotides, in the illustration of FIG. 5 codon A is represented by nucleotides AAA, codon B by nucleotides BBB, and so on.

An exemplary amino acid sequence, X Y Y X X X, is received. For this example, we assume that amino acids X and Y are associated with codons A, B, C, D, E, F, G, and H, as defined in relation to FIGS. 3, 4A, and 4B. In this example, the codon usage table of FIG. 3 has been normalized probabilistically, leading to the normalized codon usage table of FIG. 4B.In a step 501, for each amino acid, a codon is selected with a probability equal to the usage frequency associated with the codon in the normalized codon usage table. For example, for the first amino acid in the sequence, X, there is an 18% chance that codon A will be selected, a 24% chance that codon B will be selected, a 45% chance that codon D will be selected, and a 13% chance that codon F will be selected. This is because amino acid X is encoded by codons A, B, D, and F, and is thus associated with these codons in the normalized codon usage table, so the codon selected for amino acid X will be one of codons A, B, D, and F.

This process is repeated for each amino acid, using the normalized codon usage table to instruct the probability of selection of a certain codon. Thus, for the second amino acid in the sequence, Y, codon G is selected with a probability of 60% and codon H is selected with a probability of 40%. Once a codon has been selected for each amino acid, the resulting sequence of codons, made up of nucleotides, may be referred to as an optimized nucleotide sequence.

FIG. 5 is illustrative and intended only to aid in understanding the generation of an optimized sequence of nucleotides. FIG. 5 may not show the length, content, or structure of an actual received amino acid sequence or optimized nucleotide sequence, it merely diagrammatically illustrates the method.

### Generating a Plurality of Optimized Nucleotide Sequences

The generation of an optimized nucleotide sequence using the amino acid sequence and the normalized codon usage table may be performed more than once, in order to generate a list of optimized nucleotide sequences.

The list may include any number of different optimized nucleotide sequences, because the generation of an optimized nucleotide sequence is based on a probabilistic selection of codons. The list may include any number of duplicate optimized nucleotide sequences, i.e. identical optimized nucleotide sequences, again, because the generation of an optimized nucleotide sequence is based on a probabilistic selection of replacement codons. Identical optimized sequences are typically removed when generating the list of optimized nucleotide sequences.

In some embodiments, one or more, or all, of the optimized nucleotide sequences in the list of optimized nucleotide sequences are synthesized for testing by transfection, use in therapy, or for any other use of a synthesized optimized nucleotide sequence described herein.

### Filtering the List of Optimized Nucleotide Sequences

The number of optimized nucleotide sequences in the list of optimized nucleotide sequences depends at least upon the length and content of the amino acid sequence, the value of the threshold codon usage frequency, the content of the first codon usage table, and the number of times the codon optimization algorithm is run, i.e., the number of times an optimized nucleotide sequence is generated. For example, a list of optimized nucleotide sequences may comprise 10,000 or more optimized nucleotide sequences. Synthesizing and testing each optimized nucleotide sequence in the list in a cell, tissue or organism may be advantageous in some scenarios, for example, for certain algorithmic input parameters such as a relatively short amino acid sequence. Equally it may not be advantageous in certain scenarios, for example if it is desirable to reduce the complexity of the computer process or the number of sequences that are synthesized and tested in a cell, tissue, or organism. It may, therefore, be desirable to reduce the number of optimized nucleotide sequences in the list of nucleotide sequences before, e.g., synthesis. This may advantageously reduce the time it takes to synthesize every sequence in the list and the resources necessary to do so.

Accordingly, in a typical embodiment, one or more further algorithmic step(s) are performed on the list of optimized nucleotide sequences in order to filter the list, or remove optimized nucleotide sequences from the list. The one or more further algorithmic step(s) may be referred to as motif screen, GC content analysis, and codon adaptation index (CAI) analysis. It will be appreciated that although specific further algorithmic steps are described in detail herein, these may not be the only filtering steps performed, and additional steps may be performed to further filter the list of optimized nucleotide sequences within the scope of the present claims.

The inventors have found that these further algorithmic steps, and the associated motifs, ranges, and thresholds, advantageously filter the list of optimized nucleotide sequences by removing from the list sequences which are likely to be less effective than the sequences left in the list. In this way, the filtering of the list is not merely arbitrary. In other words, filtering the list down to a certain number of sequences using the methods described herein will produce an updated list of sequences containing more effective sequences than if that same certain number of sequences were randomly selected from the list. The efficiency and reduction in complexity achieved in the synthesizing process does not, therefore, come at the cost of sacrificing a large number of effective optimized nucleotide sequences. For example, the optimized nucleotide sequences generated by the methods of the invention do not contain termination signals. The absence of termination signals facilitates synthesis of full length mRNA molecules from the encoded optimized nucleotide sequences using in vitro transcription. The presence of termination signals leads to premature termination of in vitro transcription, therefore filtering the list using the methods described herein produces an updated list of sequences containing more effective sequences.

Filtering the list of optimized nucleotide sequences may be referred to as screening the list of optimized nucleotide sequences to identify and remove optimized nucleotide sequences failing to meet one or more criteria. The criteria may each relate to a certain further algorithmic step as described in detailed herein. In other words, the criteria may comprise: the optimized nucleotide sequence not containing a termination signal (a first criterion), the optimized nucleotide sequence having a guanine-cytosine content within a predetermined guanine-cytosine content range (a second criterion), the optimized nucleotide sequence having a codon adaptation index greater than a predetermined codon adaptation index threshold (a third criterion), and the optimized nucleotide sequence not having . It will be appreciated that the numbering of the criteria used is for the sake of clarity only, and is not intended to be limiting on the order of the steps, which is described in greater detail elsewhere herein.

It will be appreciated that although specific criteria are described in detail herein, these may not be the only criteria for which the optimized nucleotide sequences are screened, and additional criteria may be screened for to further filter the list of optimized nucleotide sequences within the scope of the present claims.

When screening each optimized nucleotide sequence, the entirety of the optimized nucleotide sequence may be analyzed before a determination is made as to whether it fulfils the criteria. Alternatively, each optimized nucleotide sequence may be analyzed portion by portion. A portion may be referred to as a window.

As an example, for an optimized nucleotide sequence, in the list of optimized nucleotide sequences, having a length of 600 nucleotides, a portion length may be selected at 30 nucleotides. The first 30 nucleotides of the optimized nucleotide sequence may first be analyzed for compliance with a certain criterion, i.e., nucleotides 1 to 30 of the optimized nucleotide sequence. If the first portion fails to meet the criterion, the optimized nucleotide sequence may be removed from the list of optimized nucleotide sequences.

If first portion meets the criterion, the filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found failing to meet the criterion, in which case the optimized nucleotide sequence may be removed from the list, or the whole optimized nucleotide sequence has been analyzed and no such portion has been found, in which case the filter retains the optimized nucleotide sequence in the list and may move on to the next optimized nucleotide sequence in the list. In this example, if the filter reaches the final portion of the optimized nucleotide sequence, i.e., nucleotides 571 to 600, and this final portion fulfils the criterion, the filter retains the optimized nucleotide sequence in the list and may move on to the next optimized nucleotide sequence in the list. Alternatively, and in particular, each portion may be 100 nucleotides in length.

Although the above example describes a portion by portion filter starting at the first nucleotide and proceeding to the final nucleotide, it will be appreciated that this is an example only, and the order in which the portions of the optimized nucleotide sequence are analyzed may be any order apparent to a person skilled in the art. The filter may, for example, start with a portion including the final nucleotide (in the worked example, nucleotide 600), and work back towards the first nucleotide, nucleotide 1, or may start with a portion at any position in between the first and final nucleotides.

There may be a first, final, or intermediate portion of the optimized nucleotide sequence having a different length to the other portions. This may occur, for example, if the nucleotide length of the optimized nucleotide sequence does not divide exactly by the nucleotide length of the portions.

As detailed elsewhere herein, a portion-by-portion analysis may be advantageous at least for computational efficiency, but also for a more effective identification of less desirable sequences, which may fulfil a criterion in the average, but which contain sections which do not fulfil the criterion, for example peaks or troughs of GC content or CAI score.

The optimized nucleotide sequences in the list may screened for compliance with of the one or more criteria in one of two ways: each sequence may be screened for all relevant criteria, and removed from the list if failing any one of them; or, in particular, all sequences in the list may be screened for a certain criteria, and a reduced, filtered list, screened for further criteria of interest.

### Motif Screen

In some embodiments, a motif screen filter may be applied to the list of optimized nucleotide sequences. In such embodiments, the list of optimized nucleotide sequences is analyzed to determine whether each optimized nucleotide sequence in the list contains a termination signal. The list of optimized nucleotide sequences may be the list of optimized nucleotide sequences originally generated by the codon optimization algorithm or may be a list of optimized nucleotide sequences that has already been filtered by one or more further algorithmic step(s). A list of optimized nucleotide sequences that has already been filtered, or updated, by one or more additional algorithmic step(s) may be referred to as an updated list, or most recently updated list, of optimized nucleotide sequences. Any optimized nucleotide sequence that contains one or more termination signal may be removed from the list to produce an updated list.

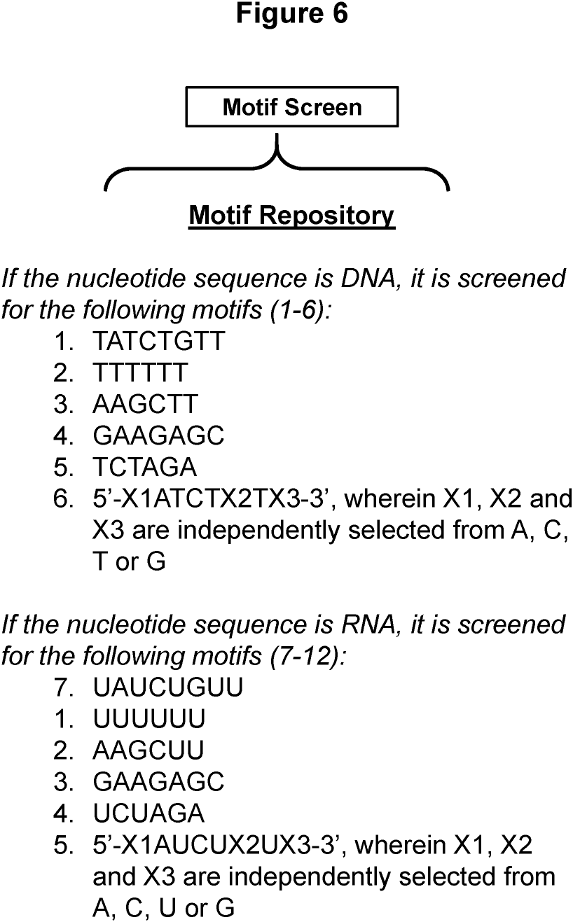

Referring to FIG. 6, the termination signal may have the following nucleotide sequences: 5′-X₁ATCTX₂TX₃-3′, wherein X₁, X₂ and X₃ are independently selected from A, C, T or G; TATCTGTT; TTTTTT; AAGCTT; GAAGAGC; TCTAGA; UAUCUGUU; UUUUUU; AAGCUU; GAAGAGC; UCUAGA; and/or 5′-X₁AUCUX₂UX₃-3′, wherein X₁, X₂ and X₃ are independently selected from A, C, U or G. The motif screen filter may determine whether each optimized nucleotide sequence contains one, some, or all of these termination signals.

Each optimized nucleotide sequence may be analyzed in its entirety, i.e., from the first nucleotide in the sequence to the last nucleotide in the sequence. In a particular embodiment, the analysis of a certain optimized nucleotide sequence may stop when the presence of a termination signal is determined in that sequence; that sequence may then be removed from the list without analyzing every one of its nucleotides. In the particular embodiment, this form of analysis may be applied to each optimized nucleotide sequence in the list. Analysis in this way can be advantageous because it is computationally efficient not to analyze an entire sequence if the presence of a termination signal in that sequence has already been determined.

Each optimized nucleotide sequence may be analyzed portion by portion, as will be described in greater detail in relation to the GC content analysis. The analysis of an optimized nucleotide sequence may halt upon a determination that a portion contains a termination signal. This may be advantageous because it is computationally efficient not to analyze an entire sequence if the presence of a termination signal in that sequence has already been determined. As for the GC content analysis to follow, the portions may or may not be overlapping, and may be of any length, for example, 5 to 300 nucleotides, or 10 to 200 nucleotides, or 15 to 100 nucleotides, or 20 to 50 nucleotides, or, in particular, 30 nucleotides or 100 nucleotdies. Each of the portions of the optimized nucleotide sequence may be the same length, or, for example, a first, final, or intermediate portion of the optimized nucleotide sequence may be of a different length to the other portions, for example if the nucleotide length of the optimized nucleotide sequence does not divide exactly by the nucleotide length of the portions.

### GC Content Analysis

In some embodiments, a guanine-cytosine (GC) content filter may be applied to the list of optimized nucleotide sequences. In such embodiments, the list of optimized nucleotide sequences is analyzed to determine a GC content of each of the optimized nucleotide sequences in the list of optimized nucleotide sequences, wherein the GC content of a sequence is the percentage of bases in the nucleotide sequence that are guanine (G) or cytosine (C). The list of optimized nucleotide sequences may be the list of optimized nucleotide sequences originally generated by the codon optimization algorithm or may be a list of optimized nucleotide sequences that has already been filtered by one or more further algorithmic step(s). A list of optimized nucleotide sequences that has already been filtered, or updated, by one or more additional algorithmic step(s) may be referred to as an updated list, or most recently updated list, of optimized nucleotide sequences. Any optimized nucleotide sequence that has a GC content falling outside a predetermined GC content range may be removed from the list to produce an updated list.

Each optimized nucleotide sequence may be analyzed in its entirety, i.e., from the first nucleotide in the sequence to the last nucleotide in the sequence. The GC content of the entire optimized nucleotide sequence may then be determined and sequences removed accordingly.

In some embodiments, only a portion of each optimized nucleotide sequence is analyzed, and the GC content of that portion determined. In such embodiments, if the GC content of the analyzed portion falls outside the predetermined GC content range, the optimized nucleotide sequence having that portion is removed from the list.

In a particular embodiment, the GC content filter is applied to each optimized nucleotide sequence portion by portion, with the filter halting and the sequence being removed if a portion is determined to have a GC content falling outside the predetermined range. Analysis in this way can be advantageous because it is computationally efficient not to analyze an entire sequence if the presence of a portion in that sequence having a GC content falling outside the predetermined GC content range has already been found.

In a particular embodiment, the portions are non-overlapping, however, in other embodiments, the portions may overlap. It will be appreciated that this particular embodiment can be performed with any length of portion, for example, 5 to 300 nucleotides, or 10 to 200 nucleotides, or 15 to 100 nucleotides, or 20 to 50 nucleotides, or, in particular, 30 nucleotides or 100 nucleotdies. In some embodiments, the predetermined GC content range may be selectable by a user. It will also be appreciated that this particular embodiment can be performed with any length of optimized nucleotide sequence.

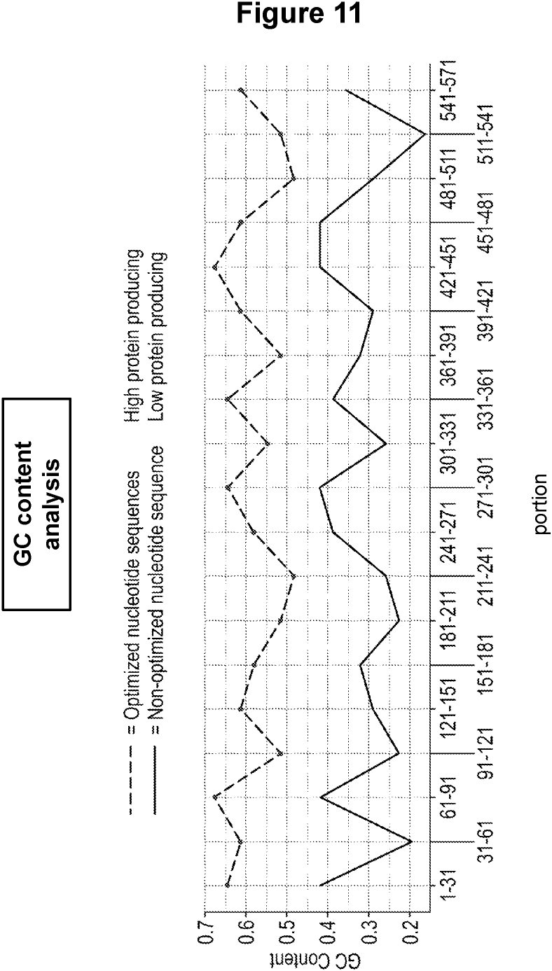

For example, analysis of the guanine-cytosine (GC) content of non-optimized and optimized nucleotide sequences can be performed on portions of the nucleotide sequence encoding EPO, wherein the guanine-cytosine (GC) content of portions of the nucleotide sequence encoding EPO is determined for adjacent non-overlapping portions 30 nucleotides in length. This exemplary analysis is illustrated in FIG. 11.

An exemplary GC content filter is described herein. It will be apparent to any person skilled in the art that this is an example only, and that the methods described herein may be performed with any length of optimized nucleotide sequence and/or portion. As an example, for an optimized nucleotide sequence, in the list of optimized nucleotide sequences, having a length of 600 nucleotides, a portion length may be selected at 30 nucleotides. The GC content filter may first analyze the first 30 nucleotides of the optimized nucleotide sequence, i.e., nucleotides 1 to 30 of the optimized nucleotide sequence. Analysis may comprise determining the number of nucleotides in the portion with are either G or C, and determining the GC content of the portion may comprise dividing the number of G or C nucleotides in the portion by the total number of nucleotides in the portion. The result of this analysis will provide a value describing the proportion of nucleotides in the portion that are G or C, and may be a percentage, for example 50%, or a decimal, for example 0.5. If the GC content of the first portion falls outside a predetermined GC content range, the optimized nucleotide sequence may be removed from the list of optimized nucleotide sequences.

If the GC content of the first portion falls inside the predetermined GC content range, the GC content filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found having a GC content falling outside the predetermined GC content range, in which case the optimized nucleotide sequence may be removed from the list, or the whole optimized nucleotide sequence has been analyzed and no such portion has been found, in which case the GC content filter retains the optimized nucleotide sequence in the list and may move on to the next optimized nucleotide sequence in the list. In this example, if the GC content filter reaches the final portion of the optimized nucleotide sequence, i.e., nucleotides 571 to 600, and this final portion has a GC content falling inside the predetermined GC content range, the GC content filter retains the optimized nucleotide sequence in the list and may move on to the next optimized nucleotide sequence in the list. Alternatively, and in particular, each portion may be 100 nucleotides in length.

Although the above example describes a portion by portion GC content filter starting at the first nucleotide and proceeding to the final nucleotide, it will be appreciated that this is an example only, and the order in which the portions of the optimized nucleotide sequence are analyzed may be any order apparent to a person skilled in the art. The GC content filter may, for example, start with a portion including the final nucleotide (in the worked example, nucleotide 600), and work back towards the first nucleotide, nucleotide 1, or may start with a portion at any position in between the first and final nucleotides.

There may be a first, final, or intermediate portion of the optimized nucleotide sequence having a different length to the other portions. This may occur, for example, if the nucleotide length of the optimized nucleotide sequence does not divide exactly by the nucleotide length of the portions.

### Codon Adaptation Index (CAI) Analysis

In some embodiments, a codon adaptation index (CAI) analysis may be performed on some or all of the optimized nucleotide sequences in the list of optimized nucleotide sequences. In such embodiments, one or more optimized nucleotide sequence in the list of optimized nucleotide sequences is analyzed to determine the CAI of each sequence, wherein CAI is a measure of codon usage bias and can take a value between 0 and 1. The list of optimized nucleotide sequences may be the list of optimized nucleotide sequences originally generated by the codon optimization algorithm or may be a list of optimized nucleotide sequences that has already been filtered by one or more further algorithmic step(s). A list of optimized nucleotide sequences that has already been filtered, or updated, by one or more additional algorithmic step(s) may be referred to as an updated list, or most recently updated list, of optimized nucleotide sequences. Any optimized nucleotide sequence having a CAI less than or equal to a predetermined CAI threshold may be removed from the list to produce an updated list.

In some embodiments, the CAI threshold is selectable by a user. In some embodiments, the CAI threshold is 0.7, 0.75, 0.85, or 0.9. In a particular embodiment, the CAI threshold is 0.8.

A CAI may be calculated, for each optimized nucleotide sequence, in any way that would be apparent to a person skilled in the art, for example as described in “The codon adaptation index--a measure of directional synonymous codon usage bias, and its potential applications” (Sharp and Li, 1987. Nucleic Acids Research 15(3), p.1281-1295); available online at https://www.ncbi.nlm.nih.gov/pmc/articles/PMC340524/.

Implementing a codon adaptation index calculation may include a method according to, or similar to, the following. For each amino acid in a sequence, a weight of each codon in a sequence may be represented by a parameter termed relative adaptiveness (wi). Relative adaptiveness may be computed from a reference sequence set, as the ratio between the observed frequency of the codon fᵢ and the frequency of the most frequent synonymous codon fⱼ for that amino acid. The codon adaptation index of a sequence may then be calculated as the geometric mean of the weight associated to each codon over the length of the sequence (measured in codons). The reference sequence set used to calculate codon adaptation index may be the same reference sequence set from which a codon usage table used with methods of the invention is derived.

As previously noted, the CAI analysis filter may be applied as a portion-by-portion analysis as detailed herein. In other words, the CAI measure of portions of each optimized nucleotide sequence may be determined, and the sequence removed from consideration (i.e. removed from the list) if any portion has a CAI less than or equal to the predetermined CAI threshold. Performing the method in this way achieves both increased computational efficiency and a more selective filter.

### Combining Further Algorithmic Steps

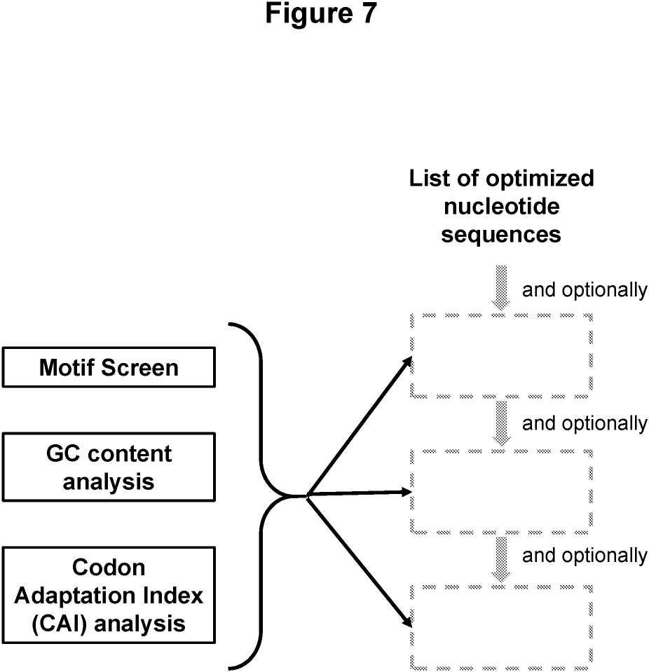

FIG. 7 illustrates that none, one, two, or three of a motif screen filter, a GC content analysis filter, and a CAI analysis filter can be applied to the list of optimized nucleotide sequences and in any order. Since each filter, if applied to the same list of optimized nucleotide sequences and with the same input parameters, has the same effect on the list, each filter may only be used once. For example, if a motif screen filter and a GC content analysis filter have been applied to the list of optimized nucleotide sequences, applying an additional motif screen filter or additional GC content analysis filter to the updated list of optimized nucleotide sequences would have no effect. This is because any sequences in the list falling foul of either filter would already have been removed. Also illustrated by FIG. 7 is that there are embodiments of the invention in which no filter is applied to the list of optimized nucleotide sequences.

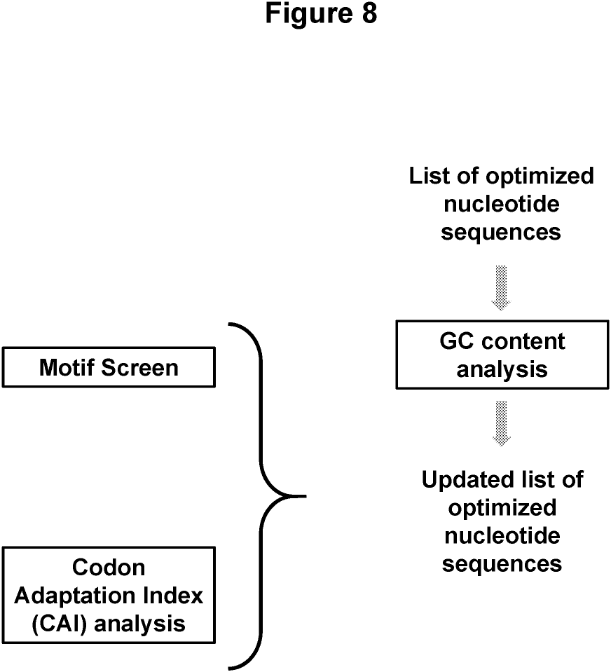

FIG. 8 illustrates an embodiment of the invention in which only one filter is applied to the list of optimized nucleotide sequences. In this embodiment, a GC content analysis filter has been selected, however it will be apparent that this is exemplary, and that, if only one filter is desired, a motif screen filter or CAI filter could alternatively be selected.

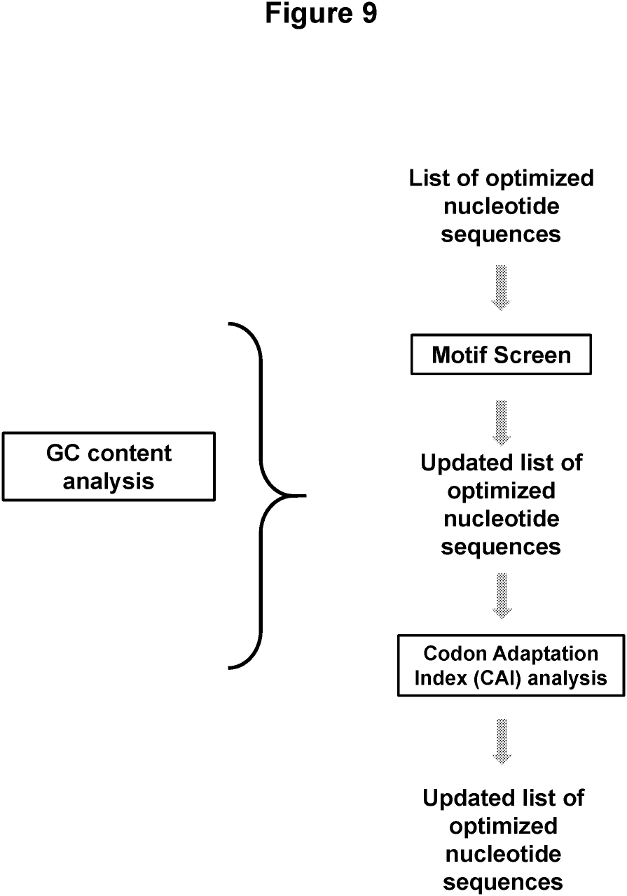

FIG. 9 illustrates an embodiment of the invention in which only two filters are applied to the list of optimized nucleotide sequences. In this embodiment, a motif screen filter and CAI analysis filter have been applied, in that order, however it will be apparent that this is exemplary, and that, if only two filters are desired, any two of a motif screen filter, GC content analysis filter, and CAI analysis filter could be applied, and in any order. In the example of FIG. 9, a motif screen filter is applied to the list of optimized nucleotide sequences to produce an updated list of optimized nucleotide sequences. Before the updated list of optimized nucleotide sequences is further filtered by a CAI analysis filter, the list may be referred to as a most recently updated list of optimized nucleotide sequences. A CAI analysis filter is then applied to the most recently updated list of optimized nucleotide sequences to produce an updated, or further updated, list of optimized nucleotide sequences.

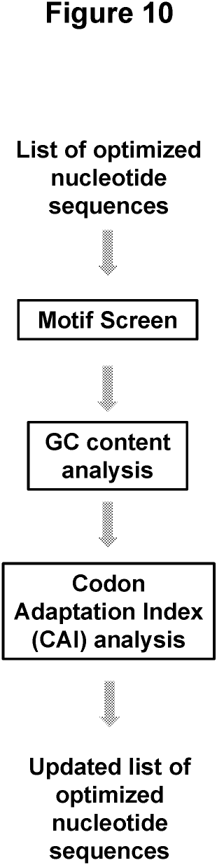

FIG. 10 illustrates a particular embodiment of the invention in which three filters are applied to the list of optimized nucleotide sequences. In this particular embodiment, a motif screen filter, GC content analysis filter, and CAI analysis filter have been applied, in that order, to produce an updated list of optimized nucleotide sequences. It will be apparent that, in alternative embodiments using three filters, the motif screen filter, GC content analysis filter, and CAI analysis filter may be applied in any order. Similarly to FIG. 9, between each filter step, i.e., between the motif screen and GC content analysis filters, and between the GC content analysis and CAI analysis filters, the list of optimized nucleotide sequences may be referred to as a most recently updated list of optimized nucleotide sequences (not shown in FIG. 10). As with the exemplary embodiments of FIGS. 8 and 9, the sequences in the updated list of optimized nucleotide sequences produced at the end of any and all filtering steps may then be synthesized according to any of the methods of synthesis described herein.

There may be a synergistic advantageous effect to filtering with more than one of the further algorithmic steps. This is achieved because the input to each further algorithmic step is the most recently updated list of optimized nucleotide sequences, i.e., may be a list of sequences which has already been filtered. This reduces the processing and time requirements to perform a further filtering step, because there are not as many sequences in the list to analyze, thereby increasing the efficiency of the method.

### Adjacent Identical Codons

In some embodiments, some or all of the optimized nucleotide sequences in the list of optimized nucleotide sequences may be analyzed to determine optimized nucleotide sequences having at least 2, for example 3 or more, adjacent identical codons. This further algorithmic step may be the only further algorithmic step, or may be performed before or after one or more of: a motif screen, a GC content analysis, and a CAI analysis. The analysis may be performed on each optimized nucleotide sequence portion-by-portion, as described in detail herein.

For example, a certain optimized nucleotide sequence may be analysed and determined to contain a section comprising: CAGCAGCAG. Such a section containing a certain repeated codon can stall transcription, so the sequence is removed from the list.

In some embodiments, an adjacency rarity threshold is used to determine rare codons, wherein codons below the adjacency rarity threshold are considered to be rare codons. Rare codons may be identified by comparing the usage frequencies in the normalized codon usage table to the adjacency rarity threshold. In this way, the adjacency rarity threshold identifies codons which had a usage greater than the threshold frequency, so as to be included in the normalized codon usage table, but are nevertheless relatively rare amongst the codons in the normalized codon usage table. In some embodiments, only rare adjacent identical codons cause the optimized nucleotide sequence to be removed from the list of optimized nucleotide sequences.

The adjacency rarity threshold may be between 10 and 50%, for example between 15 and 40 %, for example between 20 and 30%, and will depend on the threshold frequency used to normalize the codon usage table. The adjacency rarity threshold must be greater than the threshold frequency in order to have an effect, since any codon with a usage frequency below the threshold frequency will not appear in the normalized codon usage table.

Using the same example as above, but filtering only for rare adjacent identical codons, if CAG appears in the normalized codon usage table with a frequency equal to or greater than the adjacency rarity threshold, the sequence containing CAGCAGCAG will not be removed from the list. If, instead, CAG appears in the normalized codon usage table with a frequency less than the adjacency rarity threshold, the sequence containing CAGCAGCAG will be removed from the list.

A filter for adjacent identical codons, including optionally for rare adjacent identical codons, can be applied at any stage after the list of optimized nucleotide sequences has been created. In other words, a filter for adjacent identical codons, including optionally for rare adjacent identical codons, can be applied with any other further algorithmic step, with the steps being performed in any order.

### Synthesis and Expression of Optimized Nucleotide Sequences

In a further aspect, the present invention provides a method for synthesizing a nucleotide sequence, comprising: performing a computer-implemented method of the invention to generate at least one optimized nucleotide sequence; and synthesizing the at least one of the generated optimized nucleotide sequences. In vitro synthesis (also referred to commonly as “in vitro transcription”) is typically performed with a nucleic acid vector such as a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application.

In some embodiments, a synthesized optimized nucleotide sequence generated by a method of the invention is inserted in a nucleic acid vector for use in in vitro transcription. In some embodiments, the nucleic acid vector is a plasmid. The term ‘plasmid’ or ‘plasmid nucleic acid vector’ refers to a circular nucleic acid molecule, e.g., to an artificial nucleic acid molecule. A plasmid DNA in the context of the present invention is suitable for incorporating or harboring a desired nucleic acid sequence, such as a nucleic acid sequence comprising a sequence encoding an mRNA transcript and/or an open reading frame encoding at least one protein, polypeptide or peptide. Such plasmid DNA constructs/vectors may be expression vectors, cloning vectors, transfer vectors etc.

The nucleic acid vector typically comprises a sequence corresponding to (coding for) a desired mRNA transcript, or a part thereof, such as a sequence corresponding to the open reading frame and the 5′- and/or 3′UTR of an mRNA. In some embodiments, the sequence corresponding to the desired mRNA transcript may also encode a polyA-tail after the 3′ UTR so that the polyA-tail is included with the mRNA transcript. More typically in the context of the present invention, the sequence corresponding to the desired mRNA transcript consists of the 5′/3′ UTRs and the open reading frame. In some embodiments of the invention, the mRNA transcript synthesized from the nucleic acid vector during in vitro transcription does not contain a polyA tail. A polyA tail may be added to the mRNA transcript in a post-synthesis processing step.

In some embodiments, the nucleic acid vector comprises a nucleotide sequence encoding a 5′ UTR operably linked to the optimized nucleotide sequence. In particular embodiments, the 5′ UTR is different to the 5′ UTR of a naturally occurring mRNA encoding the amino acid sequence. In a specific embodiment, the 5′ UTR has the nucleotide sequence of SEQ ID NO: 19.

In some embodiments, the nucleic acid vector comprises a nucleotide sequence encoding a 3′ UTR operably linked to the optimized nucleotide sequence. In particular embodiments, the 3′ UTR is different to the 3′ UTR of a naturally occurring mRNA encoding the amino acid sequence. In a specific embodiment, the 3′ UTR has the nucleotide sequence of SEQ ID NO: 20 or SEQ ID NO: 21.

For example, the nucleotide sequence of the invention may be synthesized from a nucleic acid vector comprising an 5′ UTR, an optimized nucleotide sequence, and a 3′ UTR (and optionally one or more termination signals at the 3′ end of the optimized nucleotide sequence), to generate an mRNA comprising a 5′ UTR, an optimized nucleotide sequence, and a 3′ UTR.

In some embodiments, the nucleic acid vector comprises a promoter sequence, e.g., an RNA polymerase promoter sequence, such as a T3, T7 or SP6 RNA polymerase promoter sequence.

In some embodiments, the nucleic acid vector comprises one or more termination signals (e.g., two or three termination signals) downstream of the 3′ end of a synthesized optimized nucleotide sequence. In some embodiments, the method further comprises inserting one or more termination signals at the 3′ end of the synthesized optimized nucleotide sequences. In some embodiments, more than one termination signal is inserted, and said termination signals are separated by 10 base pairs or fewer, e.g. separated by 5-10 base pairs. The addition of one or more termination signals downstream of the optimized nucleotide sequence facilitates efficient termination of transcription as RNA is transcribed from the plasmid DNA comprising the optimized nucleotide sequence, resulting in targeted termination of in vitro transcription at the one or more termination signals and thus limiting aberrant run-on transcription. In some embodiments, the nucleic acid vector comprises more than one termination signal, e.g. two or more, three or more, or four or more. The presence of multiple termination signals enhances the efficiency of termination of in vitro transcription at the targeted site.

In some embodiments, the one or more termination signals have the following nucleotide sequence: 5′-X₁ATCTX₂TX₃-3′, wherein X₁, X₂ and X₃ are independently selected from A, C, T or G. In some embodiments, the one or more termination signals have one of the following nucleotide sequences: TATCTGTT; and/or TTTTTT; and/or AAGCTT; and/or GAAGAGC; and/or TCTAGA. In some embodiments, the one or more termination signals have the following nucleotide sequence: 5′-X₁AUCUX₂UX₃-3′, wherein X₁, X₂ and X₃ are independently selected from A, C, U or G. In some embodiments, the one or more termination signals have one of the following nucleotide sequences: UAUCUGUU; and/or UUUUUU; and/or AAGCUU; and/or GAAGAGC; and/or UCUAGA. In some embodiments, the more than one termination signals are encoded by the following nucleotide sequence: (a) 5′-X₁ATCTX₂TX₃-(Z_{N})-X₄ATCTX₅TX₆-3′ or (b) 5′-X₁ATCTX₂TX₃-(Z_{N})- X₄ATCTX₅TX₆-(Z_{M})- X₇ATCTX₈TX₉-3′, wherein X₁, X₂, X₃, X₄, X₅, X₆, X₇, X₈ and X₉ are independently selected from A, C, T or G, Z_{N} represents a spacer sequence of N nucleotides, and Z_{M} represents a spacer sequence of M nucleotides, each of which are independently selected from A, C, T of G, and wherein N and/or M are independently 10 or fewer.

Accordingly, in a particular embodiment of the invention, a plasmid DNA comprising one or more termination signals (e.g., two or three termination signals) downstream of the 3′ end of a synthesized optimized nucleotide sequence does not require linearization for in vitro transcription. Specifically, the invention makes it possible to produce mRNA transcripts from circular nucleic acid vectors such as plasmid DNA (which is typically supercoiled) using a SP6/T7 RNA polymerase for in vitro transcription.

### SP6 RNA Polymerase

In some embodiments, the mRNA is synthesized by a SP6 RNA polymerase. In some embodiments, the SP6 RNA polymerase is a naturally occurring SP6 RNA polymerase. In some embodiments, the SP6 RNA polymerase is a recombinant SP6 RNA polymerase. In some embodiments, the SP6 RNA polymerase comprises a tag. Tags can be used to facilitate protein detection or purification. In some embodiments, the tag is a his-tag, which, for example, can be used for purification with Ni-NTA affinity chromatography.

SP6 RNA Polymerase is a DNA-dependent RNA polymerase with high sequence specificity for SP6 promoter sequences. Typically, this polymerase catalyzes the 5′→3′ in vitro synthesis of RNA on either single-stranded DNA or double-stranded DNA downstream from its promoter; it incorporates native ribonucleotides and/or modified ribonucleotides into the polymerized transcript.

The sequence for bacteriophage SP6 RNA polymerase was initially described (GenBank: Y00105.1) as having the following amino acid sequence:

MQDLHAIQLQLEEEMFNGGIRRFEADQQRQIAAGSESDTAWNRRLLSELI
APMAEGIQAYKEEYEGKKGRAPRALAFLQCVENEVAAYITMKVVMDMLNT
DATLQAIAMSVAERIEDQVRFSKLEGHAAKYFEKVKKSLKASRTKSYRHA
HNVAVVAEKSVAEKDADFDRWEAWPKETQLQIGTTLLEILEGSVFYNGEP
VFMRAMRTYGGKTIYYLQTSESVGQWISAFKEHVAQLSPAYAPCVIPPRP
WRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGR
ELKEMLSPEQWQQFINWKGECARLYTAETKRGSKSAAVVRMVGQARKYSA
FESIYFVYAMDSRSRVYVQSSTLSPQSNDLGKALLRFTEGRPVNGVEALK
WFCINGANLWGWDKKTFDVRVSNVLDEEFQDMCRDIAADPLTFTQWAKAD
APYEFLAWCFEYAQYLDLVDEGRADEFRTHLPVHQDGSCSGIQHYSAMLR
DEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLE
EKEAQKAVAEGRTANKVHPFEDDRQDYLTPGAAYNYMTALIWPSISEVVK
APIVAMKMIRQLARFAAKRNEGLMYTLPTGFILEQKIMATEMLRVRTCLM
GDIKMSLQVETDIVDEAAMMGAAAPNFVHGHDASHLILTVCELVDKGVTS
IAVIHDSFGTHADNTLTLRVALKGQMVAMYIDGNALQKLLEEHEVRWMVD
TGIEVPEQGEFDLNEIMDSEYVFA (SEQ ID NO: 1)

An SP6 RNA polymerase suitable for the present invention can be any enzyme having substantially the same polymerase activity as bacteriophage SP6 RNA polymerase. Thus, in some embodiments, an SP6 RNA polymerase suitable for the present invention may be modified from SEQ ID NO: 1. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, or 60% identical or homologous to SEQ ID NO: 1. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

In some embodiments, an SP6 RNA Polymerase is encoded by a gene having the following nucleotide sequence:

ATGCAAGATTTACACGCTATCCAGCTTCAATTAGAAGAAGAGATGTTTAA
TGGTGGCATTCGTCGCTTCGAAGCAGATCAACAACGCCAGATTGCAGCAG
GTAGCGAGAGCGACACAGCATGGAACCGCCGCCTGTTGTCAGAACTTATT
GCACCTATGGCTGAAGGCATTCAGGCTTATAAAGAAGAGTACGAAGGTAA
GAAAGGTCGTGCACCTCGCGCATTGGCTTTCTTACAATGTGTAGAAAATG
AAGTTGCAGCATACATCACTATGAAAGTTGTTATGGATATGCTGAATACG
GATGCTACCCTTCAGGCTATTGCAATGAGTGTAGCAGAACGCATTGAAGA
CCAAGTGCGCTTTTCTAAGCTAGAAGGTCACGCCGCTAAATACTTTGAGA
AGGTTAAGAAGTCACTCAAGGCTAGCCGTACTAAGTCATATCGTCACGCT
CATAACGTAGCTGTAGTTGCTGAAAAATCAGTTGCAGAAAAGGACGCGGA
CTTTGACCGTTGGGAGGCGTGGCCAAAAGAAACTCAATTGCAGATTGGTA
CTACCTTGCTTGAAATCTTAGAAGGTAGCGTTTTCTATAATGGTGAACCT
GTATTTATGCGTGCTATGCGCACTTATGGCGGAAAGACTATTTACTACTT
ACAAACTTCTGAAAGTGTAGGCCAGTGGATTAGCGCATTCAAAGAGCACG
TAGCGCAATTAAGCCCAGCTTATGCCCCTTGCGTAATCCCTCCTCGTCCT
TGGAGAACTCCATTTAATGGAGGGTTCCATACTGAGAAGGTAGCTAGCCG
TATCCGTCTTGTAAAAGGTAACCGTGAGCATGTACGCAAGTTGACTCAAA
AGCAAATGCCAAAGGTTTATAAGGCTATCAACGCATTACAAAATACACAA
TGGCAAATCAACAAGGATGTATTAGCAGTTATTGAAGAAGTAATCCGCTT
AGACCTTGGTTATGGTGTACCTTCCTTCAAGCCACTGATTGACAAGGAGA
ACAAGCCAGCTAACCCGGTACCTGTTGAATTCCAACACCTGCGCGGTCGT
GAACTGAAAGAGATGCTATCACCTGAGCAGTGGCAACAATTCATTAACTG
GAAAGGCGAATGCGCGCGCCTATATACCGCAGAAACTAAGCGCGGTTCAA
AGTCCGCCGCCGTTGTTCGCATGGTAGGACAGGCCCGTAAATATAGCGCC
TTTGAATCCATTTACTTCGTGTACGCAATGGATAGCCGCAGCCGTGTCTA
TGTGCAATCTAGCACGCTCTCTCCGCAGTCTAACGACTTAGGTAAGGCAT
TACTCCGCTTTACCGAGGGACGCCCTGTGAATGGCGTAGAAGCGCTTAAA
TGGTTCTGCATCAATGGTGCTAACCTTTGGGGATGGGACAAGAAAACTTT
TGATGTGCGCGTGTCTAACGTATTAGATGAGGAATTCCAAGATATGTGTC
GAGACATCGCCGCAGACCCTCTCACATTCACCCAATGGGCTAAAGCTGAT
GCACCTTATGAATTCCTCGCTTGGTGCTTTGAGTATGCTCAATACCTTGA
TTTGGTGGATGAAGGAAGGGCCGACGAATTCCGCACTCACCTACCAGTAC
ATCAGGACGGGTCTTGTTCAGGCATTCAGCACTATAGTGCTATGCTTCGC
GACGAAGTAGGGGCCAAAGCTGTTAACCTGAAACCCTCCGATGCACCGCA
GGATATCTATGGGGCGGTGGCGCAAGTGGTTATCAAGAAGAATGCGCTAT
ATATGGATGCGGACGATGCAACCACGTTTACTTCTGGTAGCGTCACGCTG
TCCGGTACAGAACTGCGAGCAATGGCTAGCGCATGGGATAGTATTGGTAT
TACCCGTAGCTTAACCAAAAAGCCCGTGATGACCTTGCCATATGGTTCTA
CTCGCTTAACTTGCCGTGAATCTGTGATTGATTACATCGTAGACTTAGAG
GAAAAAGAGGCGCAGAAGGCAGTAGCAGAAGGGCGGACGGCAAACAAGGT
ACATCCTTTTGAAGACGATCGTCAAGATTACTTGACTCCGGGCGCAGCTT
ACAACTACATGACGGCACTAATCTGGCCTTCTATTTCTGAAGTAGTTAAG
GCACCGATAGTAGCTATGAAGATGATACGCCAGCTTGCACGCTTTGCAGC
GAAACGTAATGAAGGCCTGATGTACACCCTGCCTACTGGCTTCATCTTAG
AACAGAAGATCATGGCAACCGAGATGCTACGCGTGCGTACCTGTCTGATG
GGTGATATCAAGATGTCCCTTCAGGTTGAAACGGATATCGTAGATGAAGC
CGCTATGATGGGAGCAGCAGCACCTAATTTCGTACACGGTCATGACGCAA
GTCACCTTATCCTTACCGTATGTGAATTGGTAGACAAGGGCGTAACTAGT
ATCGCTGTAATCCACGACTCTTTTGGTACTCATGCAGACAACACCCTCAC
TCTTAGAGTGGCACTTAAAGGGCAGATGGTTGCAATGTATATTGATGGTA
ATGCGCTTCAGAAACTACTGGAGGAGCATGAAGTGCGCTGGATGGTTGAT
ACAGGTATCGAAGTACCTGAGCAAGGGGAGTTCGACCTTAACGAAATCAT
GGATTCTGAATACGTATTTGCCTAA (SEQ ID NO: 2).

A suitable gene encoding the SP6 RNA polymerase suitable in the present may be about 99%, 98%, 97%, 96%, 95%, 94%, 93%, 92%, 91%, 90%, 89%, 88%, 87%, 86%, 85%, 84%, 83%, 82%, 81%, or 80% identical or homologous to SEQ ID NO: 2.

An SP6 RNA polymerase suitable for the invention may be a commercially-available product, e.g., from Ambion, New England Biolabs (NEB), Promega, and Roche. The SP6 may be ordered and/or custom designed from a commercial source or a non-commercial source according to the amino acid sequence of SEQ ID NO: 1 or a variant of SEQ ID NO: 1 as described herein. The SP6 RNA polymerase may be a standard-fidelity polymerase or may be a high-fidelity/high-efficiency/high-capacity which has been modified to promote RNA polymerase activities, e.g., mutations in the SP6 RNA polymerase gene or post-translational modifications of the SP6 RNA polymerase itself. Examples of such modified SP6 include SP6 RNA Polymerase-Plus™ from Ambion, HiScribe SP6 from NEB, and RiboMAX™ and Riboprobe® Systems from Promega.

In some embodiments, the SP6 RNA polymerase is thermostable. In a particular embodiment, the amino acid sequence of an SP6 RNA polymerase for use with the invention contains one or more mutations relative to a wild-type SP6 polymerase that render the enzyme active at temperatures ranging from 37° C. to 56° C. In some embodiment, an SP6 RNA polymerase for use with the invention functions at an optimal temperature of 50° C. -52° C. In other embodiment, an SP6 RNA polymerase for use with the invention has a half-life of at least 60 minutes at 50° C. For example, a particularly suitable SP6 RNA polymerase for use with the invention has a half-life of between 60 minutes and 120 minutes (e.g., between 70 minutes and 100 minutes, or 80 minutes to 90 minutes) at 50° C.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., Costa et al. Frontiers in Microbiology 5 (2014): 63 and in PCT/US16/57044, the contents of which are incorporated herein by reference in their entireties. In some embodiments, a His tag is located at SP6′s N-terminus.

### SP6 Promoter

Any promoter that can be recognized by an SP6 RNA polymerase may be used in the present invention. Typically, an SP6 promoter comprises 5′ ATTTAGGTGACACTATAG-3′ (SEQ ID NO: 3). Variants of the SP6 promoter have been discovered and/or created to optimize recognition and/or binding of SP6 to its promoter. Non-limiting variants include but are not limited to : 5′-ATTTAGGGGACACTATAGAAGAG-3′; 5′-ATTTAGGGGACACTATAGAAGG-3′; 5′-ATTTAGGGGACACTATAGAAGGG-3′; 5′-ATTTAGGTGACACTATAGAA-3′; 5′-ATTTAGGTGACACTATAGAAGA-3′; 5′-ATTTAGGTGACACTATAGAAGAG-3′; 5′-ATTTAGGTGACACTATAGAAGG-3′; 5′-ATTTAGGTGACACTATAGAAGGG-3′; 5′-ATTTAGGTGACACTATAGAAGNG-3′; and 5′-CATACGATTTAGGTGACACTATAG-3′ (SEQ ID NO: 4 to SEQ ID NO: 13). Where N is used in the nucleotide sequences, N is A, C, T or G.

In addition, a suitable SP6 promoter for the present invention may be about 95%, 90%, 85%, 80%, 75%, or 70% identical or homologous to any one of SEQ ID NO: 4 to SEQ ID NO: 13. Moreover, an SP6 promoter suitable in the present invention may include one or more additional nucleotides 5′ and/or 3′ to any of the promoter sequences described herein.

### T7 RNA Polymerase

In some embodiments, the mRNA is synthesized by a T7 RNA polymerase.

T7 RNA Polymerase is a DNA-dependent RNA polymerase with high sequence specificity for T7 promoter sequences. Typically, this polymerase catalyzes the 5′→3′ in vitro synthesis of RNA on either single-stranded DNA or double-stranded DNA downstream from its promoter; it incorporates native ribonucleotides and/or modified ribonucleotides into the polymerized transcript.

In some embodiments, the T7 RNA polymerase is thermostable. In a particular embodiment, the amino acid sequence of a T7 RNA polymerase for use with the invention contains one or more mutations relative to a wild-type T7 polymerase that render the enzyme active at temperatures ranging from 37° C. to 56° C. An example for a suitable RNA polymerase is Hi-T7® RNA Polymerase from NEB. In some embodiment, a T7 RNA polymerase for use with the invention functions at an optimal temperature of 50° C. -52° C. In other embodiment, a T7 RNA polymerase for use with the invention has a half-life of at least 60 minutes at 50° C. For example, a particularly suitable T7 RNA polymerase for use with the invention has a half-life of between 60 minutes and 120 minutes (e.g., between 70 minutes and 100 minutes, or 80 minutes to 90 minutes) at 50° C.

### T7 Promoter

Any promoter that can be recognized by a T7 RNA polymerase may be used in the methods described herein. Typically, a T7 promoter comprises

5′-TAATACGACTCACTATAG-3′ (SEQ ID NO: 14).

### Post-Synthesis Processing

In some embodiments, the method of the present invention further comprises a separate step of capping and/or tailing the synthesized mRNA.

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited tom7G(5′)ppp(5′)(2′OMeG), m7G(5′)ppp(5′)(2′OMeA), m7(3′OMeG)(5′)ppp(5′)(2′OMeG), m7(3′OMeG)(5′)ppp(5′)(2′OMeA), m7G(5′)ppp (5′(A,G(5′)ppp(5′)A and G(5′)ppp(5′)G. In a specific embodiment, the cap structure is m7G(5′)ppp(5′)(2′OMeG). Additional cap structures are described in published U.S. Application No. US 2016/0032356 and U.S. Provisional Application 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly-A or poly-C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly(A) and poly(C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

In some embodiments, capping and tailing occurs during in vitro transcription.

### MRNA Synthesis Reaction Mixture Conditions

In some embodiments, the concentration of the RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the RNA polymerase is from about 10 to 50 nM, 20 to 50 nM, or 30 to 50 nM. A concentration of 100 to 10000 Units/ml of the RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

In a particular embodiment, the concentration of each rNTP in a reaction mixture is optimized based on the frequency of each nucleic acid in the nucleic acid sequence that encodes a given mRNA transcript. Specifically, such a sequence-optimized reaction mixture comprises a ratio of each of the four rNTPs (e.g., ATP, GTP, CTP and UTP) that corresponds to the ratio of these four nucleic acids (A, G, C and U) in the mRNA transcript.

In some embodiments, a start nucleotide is added to the reaction mixture before the start of the in vitro transcription. A start nucleotide is a nucleotide which corresponds to the first nucleotide of the mRNA transcript (+1 position). The start nucleotide may be especially added to increase the initiation rate of the RNA polymerase. The start nucleotide can be a nucleoside monophosphate, a nucleoside diphosphate, a nucleoside triphosphate. The start nucleotide can be a mononucleotide, a dinucleotide or a trinucleotide. In embodiments where the first nucleotide of the mRNA transcript is a G, the start nucleotide is typically GTP or GMP. In a specific embodiment, the start nucleotide is a cap analog. The cap analog may be selected from the group consisting of G[5′]ppp[5′]G, m⁷G[5′]ppp[5′]G, m₃^{2,2,7}G[5′]ppp[5′]G, m₂^{7,3′-O}G[5′]ppp[5′]G (3′-ARCA), m₂^{7,2’-O}GpppG (2′-ARCA), m₂^{7,2’-O} GppspG D1 (β-S-ARCA D1) and m₂^{7,2’-O}GppspG D2 (β-S-ARCA D2).

In specific embodiments, the first nucleotide of the RNA transcript is G, the start nucleotide is a cap analog of G and the corresponding rNTP is GTP. In such embodiments, the cap analog is present in the reaction mixture in an excess in comparison to GTP. In some embodiments, the cap analog is added with an initial concentration in the range of about 1 mM to about 20 mM, about 1 mM to about 17.5 mM, about 1 mM to about 15 mM, about 1 mM to about 12.5 mM, about 1 mM to about 10 mM, about 1 mM to about 7.5 mM, about 1 mM to about 5 mM or about 1 mM to about 2.5 mM.

More typically in the context of the present invention, a cap structure such as a cap analog is added to the mRNA transcripts obtained during in vitro transcription only after the mRNA transcripts have been synthesized, e.g., in a post-synthesis processing step. Typically, in such embodiments, the mRNA transcripts are first purified (e.g., by tangential flow filtration) before a cap structure is added.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

DNA template (e.g., as described above and in an amount/concentration sufficient to provide a desired amount of RNA), the RNA polymerase reaction buffer, and RNA polymerase are combined to form the reaction mixture. The reaction mixture is incubated at between about 37° C. and about 56° C. for thirty minutes to six hours, e.g., about sixty to about ninety minutes. In some embodiments, incubation takes place at about 37° C. to about 42° C. In other embodiment, incubation takes place at about 43° C. to about 56° C., e.g. at about 50° C. to about 52° C. As demonstrated herein, the yield of accurately terminated mRNA transcripts obtained in an in vitro transcription reaction can be increased significantly by including one or more termination signals described herein at the end of a DNA sequence encoding an mRNA transcript of interest and performing the reaction with a template including the DNA sequences at a temperature between about 50° C. to about 52° C.

In some embodiments, about 5 mM NTPs, about 0.05 mg/mL RNA polymerase, and about 0.1 mg/ml DNA template in a suitable RNA polymerase reaction buffer (final reaction mixture pH of about 7.5) is incubated at about 37° C. to about 42° C. for sixty to ninety minutes. In other embodiments, about 5 mM NTPs, about 0.05 mg/mL RNA polymerase, and about 0.1 mg/ml DNA template in a suitable RNA polymerase reaction buffer (final reaction mixture pH of about 7.5) is incubated at about 50° C. to about 52° C. for sixty to ninety minutes.

In some embodiments, a reaction mixture contains a double stranded DNA template with an RNA polymerase-specific promoter, RNA polymerase, RNase inhibitor, pyrophosphatase, 29 mM NTPs, 10 mM DTT and a reaction buffer (when at 10x is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10x is 100 mM Tris-HCl, 5 mM MgCl₂ and 25 mM CaCl₂, pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01 - 0.5 mg/ml, and RNA polymerase at a concentration ranging from 0.01 - 0.1 mg/ml, e.g., the reaction mixture comprises NTPs at a concentration of 5 mM, the DNA template at a concentration of 0.1 mg/ml, and the RNA polymerase at a concentration of 0.05 mg/ml.

### Nucleotides

Various naturally-occurring or modified nucleosides may be used to produce mRNA according to the present invention. In some embodiments, an mRNA transcript in accordance with the invention is synthesized with natural nucleosides (i.e., adenosine, guanosine, cytidine, uridine). In other embodiments, an mRNA transcript in accordance with the invention is synthesized with natural nucleosides (e.g., adenosine, guanosine, cytidine, uridine) and one or of the following: nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methylcytidine (“5mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed U.S. Pat. Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

### Transfection and Screening of Optimized Nucleotide Sequences in Cells

In some embodiments, the method of the present invention further comprises transfecting the synthesized optimized nucleotide sequence into a cell either in vivo or in vitro. In some embodiments, the expression level of the protein encoded by the synthesized optimized nucleotide sequence is determined. In some embodiments, the method further comprises synthesizing a reference nucleotide sequence and at least one synthesized optimized nucleotide sequence generated in accordance with a method of the invention, and contacting each nucleotide sequence with a separate cell or organism. In a typical embodiment, the cell or organism contacted with the at least one synthesized optimized nucleotide sequence produces an increased yield of the protein encoded by the optimized nucleotide sequence compared to the yield of the protein encoded by the reference nucleotide sequence produced by the cell or organism contacted with the synthesized reference nucleotide sequence. The reference nucleotide sequence may be: (a) a naturally occurring nucleotide sequence encoding the amino acid sequence; or (b) a nucleotide sequence encoding the amino acid sequence generated by a method other than a method of the present invention.

It may be desirable to verify that the synthesized optimized nucleotide sequences generated according to the methods of the present invention increase the expression of the encoded protein when transfected into a cell. Methods well-known in the art, such as western blotting, are suitable to experimentally verify that the codon optimization of said nucleotide sequence results in increased expression and production of the encoded protein. Furthermore, multiple synthesized optimized nucleotide sequences generated by the methods of the present invention can be screened to identify the optimized nucleotide sequence(s) which generate(s) the highest protein yield. In some embodiments, the expression level of the protein encoded by the synthesized optimized nucleotide sequence is increased at least 2-fold, e.g., at least 3-fold or 4-fold.

In some embodiments, the functional activity of the protein encoded by the synthesized optimized nucleotide sequence is determined. The functional activity of the protein encoded by the optimized nucleotide sequence can be determined using a range of well-established methods. These methods may vary depending on the properties of the encoded protein of interest. In the context of codon optimization, it may be important to experimentally verify the functional activity of the protein encoded by the synthesized optimized nucleotide sequence(s) in vitro or in vivo to ensure that expression of said encoded protein(s) produce the desired functional effect(s). For example, an enzyme activity assay may be used to determine the functional enzymatic activity of an enzyme encoded by an optimized nucleotide sequence in cells. For example, an Ussing epithelial voltage clamp assay can be used to assess the activity of human cystic fibrosis transmembrane conductance regulator (hCFTR) protein expressed from an mRNA encoding a codon-optimized hCFTR sequence generated with the methods of the invention. This assay monitors the chloride transport function of epithelial cells transfected with the hCFTR mRNA.

### Therapeutic Applications

The invention provides a synthesized optimized nucleotide sequence generated according to a method of the invention for use in therapy.

In the field of mRNA therapy, codon optimization can be used to increase expression of a functional protein encoded by mRNA in a target cell, thereby correcting protein deficiency in various disorders, including cystic fibrosis (CF), primary ciliary dyskinesia (PCD), pulmonary arterial hypertension (PAH), and idiopathic pulmonary fibrosis (IPF).

In certain aspects of the invention, the optimized nucleotide sequence encodes human cystic fibrosis transmembrane conductance regulator (hCFTR) protein:

MQRSPLEKASVVSKLFFSWTRPILRKGYRQRLELSDIYQIPSVDSADNLS
EKLEREWDRELASKKNPKLINALRRCFFWRFMFYGIFLYLGEVTKAVQPL
LLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHF
VWIAPLQVALLMGLIWELLQASAFCGLGFLIVLALFQAGLGRMMMKYRDQ
RAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAV
TRQFPWAVQTWYDSLGAINKIQDFLQKQEYKTLEYNLTTTEVVMENVTAF
WEEGFGELFEKAKQNNNNRKTSNGDDSLFFSNFSLLGTPVLKDINFKIER
GQLLAVAGSTGAGKTSLLMVIMGELEPSEGKIKHSGRISFCSQFSWIMPG
TIKENIIFGVSYDEYRYRSVIKACQLEEDISKFAEKDNIVLGEGGITLSG
GQRARISLARAVYKDADLYLLDSPFGYLDVLTEKEIFESCVCKLMANKTR
ILVTSKMEHLKKADKILILHEGSSYFYGTFSELQNLQPDFSSKLMGCDSF
DQFSAERRNSILTETLHRFSLEGDAPVSWTETKKQSFKQTGEFGEKRKNS
ILNPINSIRKFSIVQKTPLQMNGIEEDSDEPLERRLSLVPDSEQGEAILP
RISVISTGPTLQARRRQSVLNLMTHSVNQGQNIHRKTTASTRKVSLAPQA
NLTELDIYSRRLSQETGLEISEEINEEDLKECFFDDMESIPAVTTWNTYL
RYITVHKSLIFVLIWCLVIFLAEVAASLVVLWLLGNTPLQDKGNSTHSRN
NSYAVIITSTSSYYVFYIYVGVADTLLAMGFFRGLPLVHTLITVSKILHH
KMLHSVLQAPMSTLNTLKAGGILNRFSKDIAILDDLLPLTIFDFIQLLLI
VIGAIAVVAVLQPYIFVATVPVIVAFIMLRAYFLQTSQQLKQLESEGRSP
IFTHLVTSLKGLWTLRAFGRQPYFETLFHKALNLHTANWFLYLSTLRWFQ
MRIEMIFVIFFIAVTFISILTTGEGEGRVGIILTLAMNIMSTLQWAVNSS
IDVDSLMRSVSRVFKFIDMPTEGKPTKSTKPYKNGQLSKVMIIENSHVKK
DDIWPSGGQMTVKDLTAKYTEGGNAILENISFSISPGQRVGLLGRTGSGK
STLLSAFLRLLNTEGEIQIDGVSWDSITLQQWRKAFGVIPQKVFIFSGTF
RKNLDPYEQWSDQEIWKVADEVGLRSVIEQFPGKLDFVLVDGGCVLSHGH
KQLMCLARSVLSKAKILLLDEPSAHLDPVTYQIIRRTLKQAFADCTVILC
EHRIEAMLECQQFLVIEENKVRQYDSIQKLLNERSLFRQAISPSDRVKLF
PHRNSSKCKSKPQIAALKEETEEEVQDTRL(SEQ ID NO: 15)

In one particular embodiment, an optimized nucleotide sequence encoding hCFTR protein in accordance with the invention shares at least 85%, 88%, 90%, 95%, 96%, 97%, 98%, or 99% identity to SEQ ID NO: 26 and encodes a CFTR protein having an amino acid sequence of SEQ ID NO: 15. In a specific embodiment, an optimized nucleotide sequence encoding hCFTR protein in accordance with the invention is SEQ ID NO: 26. In one particular embodiment, an optimized nucleotide sequence encoding hCFTR protein in accordance with the invention shares at least 85%, 88%, 90%, 95%, 96%, 97%, 98%, or 99% identity to SEQ ID NO: 27 and encodes a hCFTR protein having an amino acid sequence of SEQ ID NO: 15. In a specific embodiment, an optimized nucleotide sequence encoding hCFTR protein in accordance with the invention is SEQ ID NO: 27. In one particular embodiment, an optimized nucleotide sequence encoding hCFTR protein in accordance with the invention shares at least 85%, 88%, 90%, 95%, 96%, 97%, 98%, or 99% identity to SEQ ID NO: 28 and encodes a hCFTR protein having an amino acid sequence of SEQ ID NO: 15. In a specific embodiment, an optimized nucleotide sequence encoding hCFTR protein in accordance with the invention is SEQ ID NO: 28.

In certain aspects, the invention provides a nucleic acid comprising an optimized nucleotide sequence encoding hCFTR protein in accordance with the invention. In particular embodiments, the invention provides an mRNA comprising an optimized nucleotide sequence encoding hCFTR protein in accordance with the invention. In some embodiments, an mRNA comprising an optimized nucleotide sequence encoding hCFTR protein in accordance with the invention also contains 5′ and 3′ UTR sequences. Exemplary 5′ and 3′ UTR sequences are shown below:

Exemplary 5′ UTR SequenceGGACAGAUCGCCUGGAGACGCCAUC
CACGCUGUUUUGACCUCCAUAGAAGACACCGGGACCGAUCCAGCCUCCGC
GGCCGGGAACGGUGCAUUGGAACGCGGAUUCCCCGUGCCAAGAGUGACUC
ACCGUCCUUGACACG (SEQ ID NO: 16)

Exemplary 3′ UTR SequenceCGGGUGGCAUCCCUGUGACCCCUCC
CCAGUGCCUCUCCUGGCCCUGGAAGUUGCCACUCCAGUGCCCACCAGCCU
UGUCCUAAUAAAAUUAAGUUGCAUCAAGCU (SEQ ID NO: 17)

or

GGGUGGCAUCCCUGUGACCCCUCCCCAGUGCCUCUCCUGGCCCUGGAAGU
UGCCACUCCAGUGCCCACCAGCCUUGUCCUAAUAAAAUUAAGUUGCAUCA
AAGCU (SEQ ID NO: 18)

Synthesized optimized nucleotide sequences generated according to a method of the invention also find use in mRNA vaccines. In the context of prophylactic mRNA vaccines, codon optimization can be used to maximize expression of a recombinant antigen encoded by mRNA delivered to a subject for optimal antigen activity, thereby generating protective immunity against a pathogen.

Similarly, in the field of cancer immunotherapy, codon optimization can be used to maximize expression of a recombinant tumor neoantigen encoded by an mRNA delivered to a subject, thereby generating an adaptive immune response against aberrant tumor cells expressing the neoantigen.

### Biotechnology Applications

In the field of biotechnology, specifically in the context of manufacturing recombinant proteins, codon optimization can be used to increase production of a protein of interest within a host cell such as a bacterial, yeast, insect, plant, or mammalian cell.

For example, the method of the present invention can be used to optimize protein expression yield of recombinant insulin protein produced in E. coli. Expression of recombinant proteins can also occur, for example, within a host cell, or in a cell-free protein extract suitable for protein expression. Codon optimization can also be used to increase production of industrially useful enzymes, suitable for use in biotechnology, manufacturing, diagnostics, and/or research.

### EXAMPLES

The following examples are included for illustrative purposes only and are not intended to limit the scope of the invention.

### Example 1 Generating Optimized Nucleotide Sequences

This example illustrates a process that results in optimized nucleotide sequences in accordance with the invention that are optimized to yield full-length transcripts during in vitro synthesis and result in high levels of expression of the encoded protein.

The process combines the codon optimization method of FIG. 1 with a sequence of filtering steps illustrated in FIG. 10 to generate a list of optimized nucleotide sequences. Specifically, as illustrated in FIG. 1, the process receives an amino acid sequence of interest and a first codon usage table which reflects the frequency of each codon in a given organism (namely human codon usage preferences in the context of the present example). The process then removes codons from the first codon usage table if they are associated with a codon usage frequency which is less than a threshold frequency (10%). The codon usage frequencies of the codons not removed in the first step are normalized to generate a normalized codon usage table.

Normalizing the codon usage table involves re-distributing the usage frequency value for each removed codon; the usage frequency for a certain removed codon is added to the usage frequencies of the other codons with which the removed codon shares an amino acid. In this example, the re-distribution is proportional to the magnitude of the usage frequencies of the codons not removed from the table, and may be performed according to the exemplary method as described in relation to FIGS. 3 and 4B. The process uses the normalized codon usage table to generate a list of optimized nucleotide sequences. Each of the optimized nucleotide sequences encode the amino acid sequence of interest.

As illustrated in FIG. 10, the list of optimized nucleotide sequences is further processed by applying a motif screen filter, guanine-cytosine (GC) content analysis filter, and codon adaptation index (CAI) analysis filter, in that order, to generate an updated list of optimized nucleotide sequences. The motif screen filter illustrated in FIG. 6 is used to remove sequences that could impede transcription or translation. The GC content analysis filter performs the process as illustrated in FIG. 11.

As illustrated in following examples, this process results in optimized nucleotide sequences encoding the amino acid sequence of interest. The nucleotide sequences yield full-length transcripts during in vitro synthesis and result in high levels of expression of the encoded protein (see Examples 2 and 3). As shown in Example 4, the expressed protein is fully functional.

### Example 2. Codon Optimization to Generate Nucleotide Sequences With a High CAI Score Improves Protein Yield.

This example demonstrates that codon-optimized protein coding sequences with a codon adaptation index (CAI) of about 0.8 or higher outperform codon-optimized protein coding sequences with a CAI below 0.8.

Codon optimization was performed on a wild-type amino acid sequence of human erythropoietin (hEPO). hEPO is a protein hormone secreted by the kidney in response to low cellular oxygen levels (hypoxia). hEPO is essential for erythropoiesis, the production of red blood cells. Recombinant hEPO is commonly used in the treatment of anemia, a condition characterized by a low red blood cell or hemoglobin count, which can occur in subjects with chronic kidney disease or in subjects undergoing cancer chemotherapy.

Using different codon optimization algorithms, a total of 5 new codon-optimized nucleotide sequences encoding hEPO (#1 through #5) were generated. Nucleotide sequences #4 and #5 were generated according to a method of the present invention as illustrated in Example 1. As a reference, a nucleotide sequence with a codon-optimized hEPO coding sequence was provided that had previously been validated experimentally both in vitro and in vivo. The reference nucleotide sequence (SEQ ID NO: 19) had been found to provide superior protein yield relative to the wild-type nucleotide sequence and other codon-optimized nucleotide sequences encoding the hEPO protein. The characteristics of each of the 5 nucleotide sequences in terms of CAI, GC content, codon frequency distribution (CFD) as well as the presence of negative CIS elements and negative repeat elements is summarized in Table 1.

TABLE 1

| Nucleotide Sequence | SEQ ID NO: | CAI | GC content % | CFD % | Negative CIS elements | Negative repeat elements |
|---|---|---|---|---|---|---|
| Reference | 19 | 0.79 | 61.06% | 3% | 0 | 0 |
| #1 | 20 | 0.69 | 54.12% | 2% | 0 | 0 |
| #2 | 21 | 0.76 | 56.23% | 1% | 0 | 0 |
| #3 | 22 | 0.90 | 57.28% | 0% | 0 | 0 |
| #4 | 23 | 0.89 | 60.95% | 0% | 0 | 0 |
| #5 | 24 | 0.86 | 59.56% | 0% | 0 | 0 |

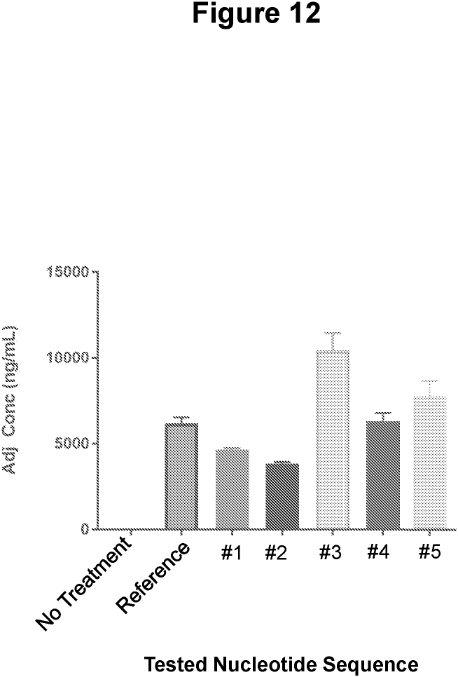

In order to test the protein yield from each of the codon-optimized sequences, 6 nucleic acid vectors were prepared each comprising an expression cassette that contained one of the 6 nucleotide sequences encoding the hEPO protein flanked by identical 3′ and 5′ untranslated sequences (3′ and 5′ UTRs) and preceded by an RNA polymerase promoter. These nucleic acid vectors served as templates for in vitro transcription reactions to provide 6 batches of mRNA containing the 6 codon-optimized nucleotide sequences (reference and nucleotide sequences #1 through #5). Capping and tailing was performed separately. Each of the capped and tailed mRNAs were separately transfected into a cell line (HEK293). Expression levels of the encoded hEPO protein was assessed by ELISA. The results of this experiment are summarized in FIG. 12.

As can be seen from FIG. 12, the highest level of expression was observed with nucleotide sequence #3 (SEQ ID NO: 22), which yielded nearly twice as much hEPO protein as the experimentally validated reference nucleotide sequence. A trend towards higher protein yield could be observed for sequences depending on their CAI (cf. Table 1). Nucleotide sequence #3 with the highest protein yield had the highest CAI. The second and third highest yielding nucleotide sequences #4 (SEQ ID NO: 23) and #5 (SEQ ID NO: 24) had the second and third highest CAI. The lowest performing nucleotide sequences #1 (SEQ ID NO: 20) and #2 (SEQ ID NO: 21) also had the lowest CAI. Incidentally, these were also the nucleotide sequences with the lowest GC content. However, GC content alone was not determinative. The reference nucleotide sequence had the highest GC content (61%) of all tested codon-optimized sequences, but did not perform as well as nucleotide sequences #3, #4 and #5, all of which had a lower GC content. Notably, the lowest performing nucleotide sequences #1 and #2 also had a higher CFD.

Taken together, the data in this example demonstrate that codon optimization of a therapeutically relevant nucleotide sequence to achieve a CAI of about 0.8 or higher results in greater protein yield than, e.g., codon optimization to achieve a nucleotide sequence with the highest possible GC content.

### Example 3. Codon Optimization of the CFTR mRNA Sequence to Increase CAI Leads To Higher Protein Expression

This example confirms that codon-optimized protein coding sequences with a codon adaptation index (CAI) of about 0.8 or higher outperform codon-optimized protein coding sequences with a CAI below 0.8.

The hEPO protein tested in Example 1 is a relatively short polypeptide whose amino acid sequence is encoded by a sequence of 495 nucleotides. To determine whether the findings in Example 1 also apply to much longer nucleotide sequences encoding a large protein, codon optimization was performed on the human cystic fibrosis transmembrane conductance regulator (hCFTR). hCFTR is encoded by a sequence of 4440 nucleotides, i.e., its sequence is about 10 times longer than the coding sequence of hEPO.

Mutations in the gene encoding the hCFTR protein cause cystic fibrosis (CF), the most common genetic disease in the Caucasian population. It is characterized by abnormal transport of chloride and sodium ions across the epithelium, leading to thick, viscous secretions that affect most critically the lungs, and also the pancreas, liver, and intestine. mRNA encoding a codon-optimized hCFTR coding sequence is being developed as a novel therapeutic to treat CF.

Codon optimization was performed on the native hCFTR amino acid sequence according to a method of the present invention as illustrated in Example 1. Three sequences designated hCFTR #1 (SEQ ID NO: 26), hCFTR #2 (SEQ ID NO: 27) and hCFTR #3 (SEQ ID NO: 28) were selected for further analysis. As a reference, a nucleotide sequence with a hCFTR coding sequence codon-optimized with a different algorithm was provided (SEQ ID NO: 25). This reference nucleotide sequence (SEQ ID NO: 25) had previously been validated experimentally both in vitro and in vivo. The reference nucleotide sequence had been found to provide superior protein yield relative to other earlier tested codon-optimized nucleotide sequences encoding the hCFTR protein. When compared to the reference nucleotide sequence, the CAI and GC content % of the codon-optimized hCFTR #2 and hCFTR #3 sequences were significantly increased. Furthermore, their codon frequency distribution (CFD) % was 0%, compared to 6% for the reference nucleotide sequence, indicating that rare codon clusters detrimental for translation efficiency were successfully removed. Additional filtering to remove negative regulatory motifs resulted in a significant reduction in the number of negative cis-regulatory (CIS) elements in hCFTR #2 and hCFTR #3 (cf. Table 2).

TABLE 2

| Nucleotide Sequence | SEQ ID NO: | CAI | GC content % | CFD % | Negative CIS elements | Negative repeat elements |
|---|---|---|---|---|---|---|
| hCFTR Reference | 25 | 0.70 | 49.52 | 6% | 7 | 0 |
| hCFTR #1 | 26 | 0.70 | 49.59 | 6% | 7 | 0 |
| hCFTR #2 | 27 | 0.89 | 53.78 | 0% | 4 | 0 |
| hCFTR #3 | 28 | 0.89 | 53.97 | 0% | 3 | 0 |

In order to test the protein yield from each of the codon-optimized sequences, 4 nucleic acid vectors were prepared each comprising an expression cassette that contained one of the 4 nucleotide sequences encoding the hCFTR protein flanked by identical 3′ and 5′ untranslated sequences (3′ and 5′ UTRs) and preceded by an RNA polymerase promoter. These nucleic acid vectors served as templates for in vitro transcription reactions to provide 4 batches of mRNA containing the 4 codon-optimized nucleotide sequences (reference and hCFTR #1 through #3). Capping and tailing was performed separately.

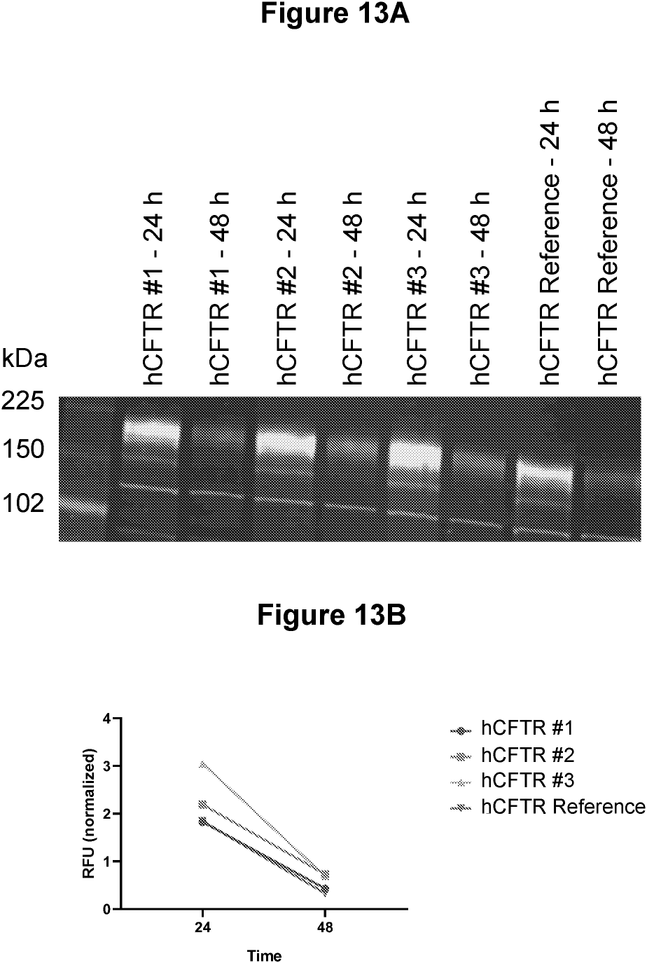

Each of the capped and tailed mRNAs were separately transfected into a cell line (HEK293). Cell lysates were collected 24 and 48 hours after transfection. Protein samples were extracted and processed for SDS-PAGE. Expression levels of the encoded hCFTR protein were assessed by Western Blot. Protein bands were developed and quantified using a LI-COR system. The protein yields were expressed as relative fluorescence units (RFU). The results of this experiment are summarized in FIG. 13. Codon optimized nucleotide sequences hCFTR #2 and hCFTR #3, which both had a CAI of 0.89, produced significantly higher yields of the encoded hCFTR protein compared to the reference nucleotide sequence and hCFTR #1, which both had a CAI of 0.7. This effect was more pronounced at the 24 hour time point (see FIG. 13B), presumably due to the relatively rapid degradation of the mRNA in HEK293cells post transfection.

The data in this example demonstrate that codon optimization of a therapeutically relevant nucleotide sequence (hCFTR) to achieve a CAI of about 0.8 or higher results in greater protein yield, in particular when also combined with optimization of its CFD and its GC content and with the removal of any negative CIS elements from the nucleic acid sequence. The data in this example also confirm that codon optimization of the hCFTR mRNA according to the methods of the present invention results in very high hCFTR protein yield in human cells in comparison to nucleotide sequences codon-optimized with a different algorithm.

### Example 4. Codon Optimization of the CFTR Nucleotide Sequence Leads to Increased Functional Activity in Cells

This example illustrates that codon optimization of the hCFTR nucleotide sequence according to a method of the present invention does not impact hCFTR functional activity in human cells.

The administration of hCFTR mRNA is intended to result in its uptake by airway epithelial cells in CF patients, followed by internalization into the cytoplasm of the target cells. Once cellular uptake is achieved, hCFTR mRNA is translated into normal hCFTR protein, which is then processed through the cell’s endogenous secretory pathway resulting in the localization of the hCFTR protein in the apical cell membrane. Through this approach, hCFTR mRNA administration produces functional hCFTR protein in the airway epithelium, thereby correcting the deficiency in functional CFTR in the lungs of the CF patients. Codon optimization of the hCFTR mRNA nucleotide sequence can increase expression of the functional hCFTR protein, which is thought to lead to a higher amount of functional hCFTR protein in the target airway epithelial cells of CF patients.

It has been reported that codon optimization can come at the cost of reduced functional activity of the encoded protein and an associated loss in efficacy as the process may remove information encoded in the nucleotide sequence that is important for controlling translation of the protein and ensuring proper folding of the nascent polypeptide chain (Mauro & Chappell, Trends Mol Med. 2014; 20(11):604-13). To test the functional activity of hCFTR protein expressed from the codon-optimized sequences generated using the codon optimization method as illustrated in Example 1, hCFTR mRNAs produced in Example 2 were tested in an Ussing chamber assay. This assay uses an epithelial voltage clamp to assess the functional activity of protein expressed from the hCFTR mRNA by monitoring the chloride transport function of epithelial cells that were transfected with said mRNA. Specifically, the functional activity of the hCFTR protein expressed from mRNAs with a control hCFTR coding sequence (SEQ ID NO: 25) or the coding sequence of hCFTR #1 (SEQ ID NO: 26), hCFTR #2 (SEQ ID NO: 27) or hCFTR #3 (SEQ ID NO: 28) was measured in Fischer rat thyroid (FRT) epithelial cells. FRT epithelial cells are commonly used as a model to study human airway epithelial cell function. FRT epithelial cells were grown in monolayers on Snapwell™ filter inserts and transfected with the 4 hCFTR mRNAs. The 4 hCFTR mRNAs were produced as described in Example 2. The control mRNA had previously been validated in this assay and was used as a reference standard.

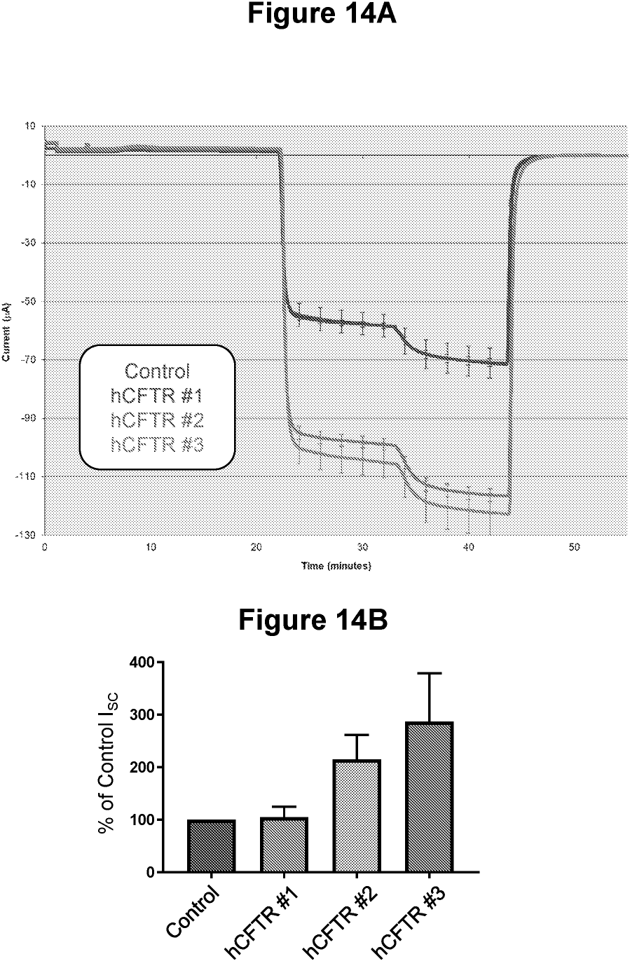

Correctly translated and localized hCFTR protein produced from a hCFTR mRNA increases the short circuit current (Isc) output within an Ussing epithelial voltage clamp apparatus when CFTR agonists (forskolin and VX-770 [Kalydeco®]) are applied. The application of CFTR antagonist CFTRinh-172 drives hCFTR into a blocked state. The Isc current polarity convention in this assay records apical-to-basolateral sodium current and basolateral-to-apical chloride current as negative values, and so if transfection with a test hCFTR mRNA generates a high negative value, it can be concluded that the encoded hCFTR protein is functional (FIG. 14A). Moreover, by transfecting equal amounts of mRNA, it can be assessed whether an mRNA produces a higher yield of hCFTR protein since protein yield and activity are correlated. Transfection of FRT epithelial cells with an mRNA having the hCFTR #1 coding sequence resulted in activity comparable to that achieved by transfection with the mRNA having the control hCFTR coding sequence (FIG. 14B). mRNAs encoding a nucleotide sequence encoding hCFTR generated by a method of the present invention resulted in significantly increased activity. Consistent with the higher protein yields observed in Example 2, hCFTR protein produced from mRNA encoding hCFTR #2 resulted in more than 2-fold higher activity relative to the control mRNA, and hCFTR protein produced from an mRNA encoding hCFTR #3 resulted in 3-fold higher activity relative to the control mRNA. This confirms that the higher protein yield resulting from hCFTR #2 and hCFTR #3 observed in Example 2 directly correlates with higher functional activity, demonstrating that codon optimization in accordance with a method of the present invention does not negatively impact the functional activity of the encoded protein.

In summary, codon optimization according to a method of the present invention results in higher expression of the encoded protein in human cells, and the expressed protein provides full functional activity in a model system that is a highly relevant model for human therapy.

### Example 5. Codon Optimization of the DNAI1 mRNA Sequence to Increase CAI Leads To Higher Parotein Expression.

The data in this example demonstrate that codon optimization of a further therapeutically relevant nucleotide sequence (DNAI1) to achieve a CAI of about 0.8 or greater results in greater protein yield in cells, in particular when also combined with optimization of its CFD and its GC content and with the removal of any negative CIS elements from the nucleic acid sequence. The data in this example also confirm that CAI values positively correlate with protein expression yield for codon-optimized mRNAs generated according to the methods of the invention.

Primary ciliary dyskinesia (PCD) is an auto recessive disorder characterized by abnormal cilia and flagella that are found in the linings of the airway, the reproductive system, and other organs and tissues. Symptoms are present as early as at birth, with breathing problems, and the affected individuals develop frequent respiratory tract infections beginning in early childhood. People with PCD also have year-round nasal congestion and chronic cough. Chronic respiratory tract infections can result in condition called bronchiectasis, which damages the passages, called bronchi, and can cause life-threatening breathing problems. Some individuals with PCD also have infertility, recurrent ear infections, abnormally placed organs within their chest and abdomen. Among several genes confirmed to be directly involved in PCD pathogenesis, a significant number of mutations are found in two genes: DNAI1 and DNAH5, encoding intermediate and heavy chains of the axonemal dynein, respectively.

mRNA encoding a codon-optimized DNAI1 coding sequence is being developed as a novel therapeutic to treat PCD.

Codon optimization was performed using the native DNAI1 amino acid sequence according to the methods of the present invention as illustrated in Example 1 to generate three sequences designated DNAI1 #1 (SEQ ID NO: 29), DNAI1 #2 (SEQ ID NO: 30), DNAI1 #3 (SEQ ID NO: 31). A codon-optimized DNAI1 sequence DNAI1 #4 (SEQ ID NO: 32) was also included as a reference. DNAI1 #4 was codon optimized but was not further processed by applying a motif screen filter, guanine-cytosine (GC) content analysis filter, and codon adaptation index (CAI) analysis filter The resulting codon-optimized nucleotide sequences generated according to the methods of the invention had CAI values of 0.8 or greater, as described in Table 3.

TABLE 3

| Nucleotide Sequence | SEQ ID NO: | CAI | GC Content % |
|---|---|---|---|
| DNAI1 #1 | 29 | 0.90 | 53.33 |
| DNAI1 #2 | 30 | 0.87 | 50.48 |
| DNAI1 #3 | 31 | 0.87 | 51.61 |
| DNAI1 #4 | 32 | 0.83 | 55.57 |

In order to test the protein yield from each of the codon-optimized sequences, 4 nucleic acid vectors were prepared each comprising an expression cassette that contained one of the 4 nucleotide sequences encoding the DNAI1 protein flanked by identical 5′ and 3′ UTRs and preceded by an RNA polymerase promoter. These nucleic acid vectors served as templates for in vitro transcription reactions to provide 4 batches of mRNA containing the 4 codon-optimized nucleotide sequences (DNAI1 #1 through #4). Capping and tailing were performed separately.

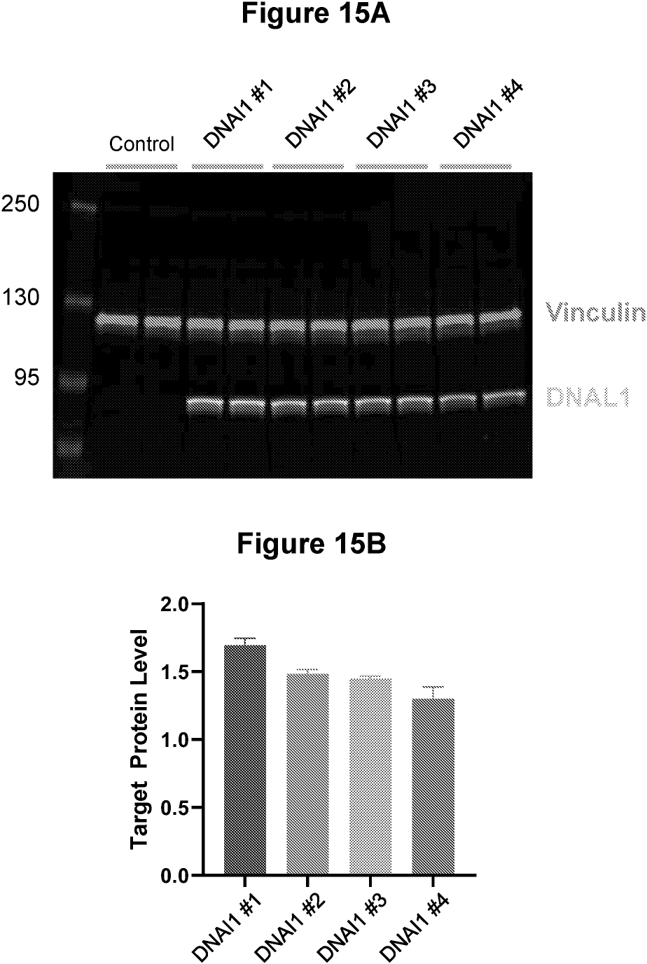

2 µg of each of the capped and tailed mRNAs was used to transfect transfected 10⁵ HEK293T cells. Untransfected HEK293T cells were also included to provide a negative control. Cell lysates were collected 24 hours after transfection, and protein samples were extracted and processed for SDS-PAGE. Two samples from each batch of cells were processed and analysed. Expression levels of the encoded DNAI1 protein were assessed by Western Blot, using an anti-DNAI1 primary antibody (αDNAI1). Expression levels of vinculin were also measured using an anti-vinculin primary antibody (αVinculin) to provide a loading control. Signals were developed and quantified using a LI-COR imaging system, and the DNAI1 protein yields normalized to vinculin were graphed in FIG. 15B as fold increase relative to a reference level achieved with an mRNA encoding a DNAL1 sequence which had not been codon-optimized. The results of this experiment are summarized in FIG. 15. Codon optimized nucleotide sequence DNAI1 #1, which had the highest CAI (0.90), produced the highest level of DNAI1 protein compared to the reference (DNAI1 #4). Codon optimized sequences DNAI1 #2 and DNAI1 #3 both had a CAI of 0.87, and produced comparable levels of DNAI1 protein despite differences in nucleotide sequence, indicating that CAI is closely associated with protein expression yield. Codon optimized sequence DNAI1 #4, with a CAI of 0.83, produced the lowest amount of protein relative to the optimized nucleotide sequences with higher CAI, but was still significantly increased relative to the reference level.

Taken together, these data indicate that for mRNAs comprising codon-optimized nucleotide sequence of the invention, a higher CAI is strongly indicative of protein expression yield, and also show that different codon-optimized nucleotide sequences with similar CAI values produce similar levels of the encoded protein, in cells.

### Numbered Embodiments of the Invention

1. A computer-implemented method for generating an optimized nucleotide sequence, comprising:
(i) receiving an amino acid sequence, wherein the amino acid sequence encodes a peptide, polypeptide, or protein;
(ii) receiving a first codon usage table, wherein the first codon usage table comprises a list of amino acids, wherein each amino acid in the table is associated with at least one codon and each codon is associated with a usage frequency;
(iii)removing from the codon usage table any codons associated with a usage frequency which is less than a threshold frequency;
(iv) generating a normalized codon usage table by normalising the usage frequencies of the codons not removed in step (iii); and
(v) generating an optimized nucleotide sequence encoding the amino acid sequence by selecting a codon for each amino acid in the amino acid sequence based on the usage frequency of the one or more codons associated with the amino acid in the normalized codon usage table.

2. The method according to embodiment 1, wherein normalising comprises:
(a) distributing the usage frequency of each codon associated with a first amino acid and removed in step (iii) to the remaining codons associated with the first amino acid; and
(b) repeating step (a) for each amino acid to produce the normalized codon usage table.

3. The method according to embodiment 2, wherein the usage frequency of the removed codons is distributed equally amongst the remaining codons.

4. The method according to embodiment 2, wherein the usage frequency of the removed codons is distributed amongst the remaining codons proportionally based on the usage frequency of each remaining codon.

5. The method according to any preceding embodiment, wherein selecting a codon for each amino acid comprises:
(a) identifying, in the normalized codon usage table, the one or more codons associated with a first amino acid of the amino acid sequence;
(b) selecting a codon associated with the first amino acid, wherein the probability of selecting a certain codon is equal to the usage frequency associated with the codon associated with the first amino acid in the normalized codon usage table; and
(c) repeating steps (a) and (b) until a codon has been selected for each amino acid in the amino acid sequence.

6. The method according to any preceding embodiment, wherein step (v) is performed a plurality of times to generate a list of optimized nucleotide sequences.

7. The method according to any preceding embodiment, wherein the threshold frequency is selectable by a user.

8. The method according to any preceding embodiment, wherein the threshold frequency is in the range of 5% - 30%, in particular 5%, 10%, or 15%, or 20%, or 25%, or 30%, or, in particular, 10%.

9. The method according to any one of embodiments 6 to 8, further comprising:
determining whether each optimized nucleotide sequence in the list, or most recently updated list, of optimized nucleotide sequences contains a termination signal; and
updating the list of optimized nucleotide sequences by removing any nucleotide sequence from the list, or most recently updated list, if the nucleotide sequence contains one or more termination signals.

10. The method according to embodiment 9, wherein the one or more termination signals has/have the following nucleotide sequence: 5’-X₁ATCTX₂TX₃-3’, wherein X₁, X₂ and X₃ are independently selected from A, C, T or G.

11. The method according to embodiment 10, wherein the one or more termination signal has/have one or more of the following nucleotide sequences: TATCTGTT; and/or TTTTTT; and/or AAGCTT; and/or GAAGAGC; and/or TCTAGA.

12. The method according to embodiment 9, wherein the one or more termination signals has/have the following nucleotide sequence: 5’-X₁AUCUX₂UX₃-3’, wherein X₁, X₂ and X₃ are independently selected from A, C, U or G.

13. The method according to embodiment 12, wherein the one or more termination signals has/have one of the following nucleotide sequences: UAUCUGUU; and/or UUUUUU; and/or AAGCUU; and/or GAAGAGC; and/or UCUAGA.

14. The method according to any one of embodiments 6 to 13, further comprising:
determining a guanine-cytosine content of each of the optimized nucleotide sequences in the list, or most recently updated list, of optimized nucleotide sequences, wherein the guanine-cytosine content of a sequence is the percentage of bases in the nucleotide sequence that are guanine or cytosine;
updating the list of optimized nucleotide sequences by removing any nucleotide sequence from the list, or most recently updated list, if its guanine-cytosine content falls outside a predetermined guanine-cytosine content range.

15. The method according to embodiment 14, wherein determining a guanine-cytosine content of each of the optimized nucleotide sequences comprises, for each nucleotide sequence:
determining the guanine-cytosine content of a first portion of the nucleotide sequence, and wherein updating the list of optimized nucleotide sequences comprises:
removing the nucleotide sequence if the guanine-cytosine content of the first portion falls outside the predetermined guanine-cytosine content range.

16. The method according to embodiment 15, wherein determining a guanine-cytosine content of each of the optimized nucleotide sequences further comprises, for each nucleotide sequence:
determining a guanine-cytosine content of one or more additional portions of the nucleotide sequence, wherein the additional portions are non-overlapping with each other and with the first portion, and wherein updating the list of optimized sequences comprises:
removing the nucleotide sequence if the guanine-cytosine content of any portion falls outside the predetermined guanine-cytosine content range, optionally wherein determining the guanine-cytosine content of the nucleotide sequence is halted when the guanine-cytosine content of any portion is determined to be outside the predetermined guanine-cytosine content range.

17. The method according to embodiment 15 or 16, wherein the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides, e.g., 30 nucleotides

18. The method according to embodiment 17, wherein the predetermined guanine-cytosine content range is selectable by a user.

19. The method according to embodiment 17 or 18, wherein the predetermined guanine-cytosine content range is 15% - 75%, or 40% - 60%, or, in particular 30% - 70%.

20. The method according to any one of embodiments 6 to 19, further comprising:
determining a codon adaptation index of each of the optimized nucleotide sequences in the list, or most recently updated list, of optimized nucleotide sequences, wherein the codon adaptation index of a sequence is a measure of codon usage bias and can be a value between 0 and 1;
updating the list, or most recently updated list, of optimized nucleotide sequences by removing any nucleotide sequence if its codon adaptation index is less than or equal to a predetermined codon adaptation index threshold.

21. The method according to embodiment 20, wherein the codon adaptation index threshold is selectable by a user.

22. The method according to embodiment 20 or 21, wherein the codon adaptation index threshold is 0.7, or 0.75, or 0.85, or 0.9, or, in particular, 0.8.

23. The method according to any preceding embodiment, wherein the amino acid sequence is received from a database of amino acid sequences.

24. The method according to embodiment 23, further comprising requesting the amino acid sequence from the database of amino acid sequences, wherein the amino acid sequence is received in response to the request.

25. The method according to any preceding embodiment, wherein the first codon usage table is received from a database of codon usage tables.

26. The method according to embodiment 24, further comprising requesting the first codon usage table from the database of codon usage tables, wherein the first codon usage table is received in response to the request.

27. The method according to any preceding embodiment, further comprising displaying at least one optimized nucleotide sequence on a screen.

28. A computer program comprising instructions which, when the program is executed by a computer, cause the computer to carry out the method of any preceding embodiment.

29. A data processing system comprising means for carrying out the method of any preceding embodiment.

30. A computer-readable data carrier having stored thereon the computer program of embodiment 28.

31. A data carrier signal carrying the computer program of embodiment 28.

32. A method for synthesizing a nucleotide sequence, comprising:
performing the computer-implemented method of any one of embodiments 1 to 27 to generate at least one optimized nucleotide sequence; and
synthesizing at least one of the generated optimized nucleotide sequences.

33. The method according to embodiment 32, wherein the method further comprises inserting the synthesized optimized sequence in a nucleic acid vector for use in vitro transcription.

34. The method according to embodiment 32 or 33, wherein the method further comprises inserting one or more termination signals at the 3′ end of the synthesized optimized nucleotide sequence.

35. The method according to embodiment 34, wherein the one or more termination signals are encoded by the following nucleotide sequence: 5’-X₁ATCTX₂TX₃-3’, wherein X₁, X₂ and X₃ are independently selected from A, C, T or G.

36. The method according to embodiment 34 or 35, wherein the one or more termination signals are encoded by one or more of the following nucleotide sequences: TATCTGTT; TTTTTT; AAGCTT; GAAGAGC; and/or TCTAGA.

37. The method according to any one of embodiments 34-36, wherein more than one termination signal is inserted, and said termination signals are separated by 10 base pairs or fewer, e.g. separated by 5-10 base pairs.

38. The method according to embodiment 36, wherein the more than one termination signals are encoded by the following nucleotide sequence: (a) 5′-X₁ATCTX₂TX₃-(Z_{N})-X₄ATCTX₅TX₆-3′ or (b) 5′-X₁ATCTX₂TX₃-(Z_{N})- X₄ATCTX₅TX₆-(Z_{M})- X₇ATCTX₈TX₉-3′, wherein X₁, X₂, X₃, X₄, X₅, X₆, X₇, X₈ and X₉ are independently selected from A, C, T or G, Z_{N} represents a spacer sequence of N nucleotides, and Z_{M} represents a spacer sequence of M nucleotides, each of which are independently selected from A, C, T of G, and wherein N and/or M are independently 10 or fewer.

39. The method according to any one of embodiments 33 to 38, wherein the nucleic acid vector comprises an RNA polymerase promoter operably linked to the optimized nucleotide sequence, optionally wherein the RNA polymerase promoter is a SP6 RNA polymerase promoter or a T7 RNA polymerase promoter.

40. The method according to any one of embodiments 33 to 39, wherein the nucleic acid vector is a plasmid.

41. The method according to embodiment 40, wherein the plasmid is linearized before in vitro transcription.

42. The method according to embodiment 40, wherein the plasmid is not linearized before in vitro transcription.

43. The method according to embodiment 42, wherein the plasmid is supercoiled.

44. The method according to any one of embodiments 32-43, wherein the method further comprises using at least one of the synthesized optimized nucleotide sequences in in vitro transcription to synthesize mRNA.

45. The method according to embodiment 44, wherein the mRNA is synthesized by a SP6 RNA polymerase.

46. The method according to embodiment 45, wherein the SP6 RNA polymerase is a naturally occurring SP6 RNA polymerase.

47. The method according to embodiment 45, wherein the SP6 RNA polymerase is a recombinant SP6 RNA polymerase.

48. The method according to embodiment 47, wherein the SP6 RNA polymerase comprises a tag.

49. The method according to embodiment 48, wherein the tag is a his-tag.

50. The method according to embodiment 44, wherein the mRNA is synthesized by a T7 RNA polymerase.

51. The method according to any one of embodiments 44-50, wherein the method further comprises a separate step of capping and/or tailing the synthesized mRNA.

52. The method according to any one of embodiments 44-50, wherein capping and tailing occurs during in vitro transcription.

53. The method according to any one of embodiments 44-52, wherein the mRNA is synthesized in a reaction mixture comprising NTPs at a concentration ranging from 1-10 mM each NTP, the DNA template at a concentration ranging from 0.01-0.5 mg/ml, and the SP6 RNA polymerase at a concentration ranging from 0.01-0.1 mg/ml.

54. The method according to embodiment 53, wherein the reaction mixture comprises NTPs at a concentration of 5 mM each NTP, the DNA template at a concentration of 0.1 mg/ml, and the SP6 RNA polymerase at a concentration of 0.05 mg/ml.

55. The method according to any one of embodiments 44-54, wherein the mRNA is synthesized at a temperature ranging from 37-56° C.

56. The method according to any one of embodiments 53-55, wherein the NTPs are naturally-occurring NTPs.

57. The method according to any one of embodiments 53-55, wherein the NTPs comprise modified NTPs.

58. The method according to any one of embodiments 32 to 57, wherein the method further comprises transfecting the synthesized optimized nucleotide sequence into a cell either in vitro or in vivo.

59. The method according to embodiment 58, wherein the expression level of the protein encoded by the synthesized optimized nucleotide sequence in transfected cell is determined.

60. The method according to embodiment 58 or 59, wherein the functional activity of the protein encoded by the synthesized optimized nucleotide sequence is determined.

61. The method according to any one of embodiments 1 to 27, further comprising synthesizing a reference nucleotide sequence encoding the amino acid sequence and the at least one optimized nucleotide sequence according to the method of any one of embodiments 32 to 60, and contacting the reference nucleotide sequence and the at least one optimized nucleotide sequence with a separate cell or organism, wherein the cell or organism contacted with the at least one synthesized optimized nucleotide sequence produces an increased yield of the protein encoded by the optimized nucleotide sequence compared to the yield of the protein encoded by the reference nucleotide sequence produced by the cell or organism contacted with the synthesized reference nucleotide sequence.

62. The method of any one of embodiments 32 to 60, wherein the method further comprises producing a therapeutic composition comprising an mRNA encoding a therapeutic peptide, polypeptide, or protein for use in the delivery to or treatment of a subject.

63. The method of embodiment 62, wherein the mRNA encodes cystic fibrosis transmembrane conductance regulator (CFTR) protein.

64. The method according to any one of embodiments 1 to 27, wherein the at least one optimized nucleotide sequence, when synthesized, is configured to increase the expression of the protein encoded by the at least one optimized nucleotide sequence compared to the expression of the protein encoded by the reference nucleotide sequence, when synthesized.

65. The method of any one of embodiments 61 to 64, wherein the reference nucleotide sequence is (a) a naturally occurring nucleotide sequence encoding the amino acid sequence or (b) a nucleotide sequence encoding the amino acid sequence generated by a method other than the method according to any one of embodiments 1 to 27.

66. A synthesized optimized nucleotide sequence generated according to the methods of any one of embodiments 32 to 57 and 62 to 65 for use in therapy.

67. A method of treatment comprising administering the synthesized optimized nucleotide sequence generated according to the method of any one of embodiments 32 to 57 and 62 to 65 to a human subject in need of such treatment.

68. An in vitro synthesized nucleic acid comprising an optimized nucleotide sequence consisting of codons associated with a usage frequency which is greater than or equal to 10%; wherein the optimized nucleotide sequence:
(i) does not contain a termination signal having one of the following nucleotide sequences:
   5′-X₁AUCUX₂UX₃-3′, wherein X₁, X₂ and X₃ are independently selected from A, C, U or G; and 5′-X₁AUCUX₂UX₃-3′, wherein X₁, X₂ and X₃ are independently selected from A, C, U or G;
(ii) does not contain any negative cis-regulatory elements and negative repeat elements; and
(iii) has a codon adaptation index greater than 0.8;
wherein, when divided into non-overlapping 30 nucleotide-long portions, each portion of the optimized nucleotide sequence has a guanine cytosine content range of 30% - 70%.

69. The in vitro synthesized nucleic acid of embodiment 67, wherein the optimized nucleotide sequence does not contain a termination signal having one of the following sequences: TATCTGTT; TTTTTT; AAGCTT; GAAGAGC; TCTAGA; UAUCUGUU; UUUUUU; AAGCUU; GAAGAGC; UCUAGA.

70. The in vitro synthesized nucleic acid of embodiment 68 or 69, wherein the nucleic acid is mRNA.

71. The in vitro synthesized nucleic acid of any one of embodiments 68 to 70 for use in therapy.

1. A computer-implemented method for generating an optimized nucleotide sequence, comprising:
(i) receiving an amino acid sequence, wherein the amino acid sequence encodes a peptide, polypeptide, or protein;
(ii) receiving a first codon usage table, wherein the first codon usage table comprises a list of amino acids, wherein each amino acid in the table is associated with at least one codon and each codon is associated with a usage frequency;
(iii) removing from the codon usage table any codons associated with a usage frequency which is less than a threshold frequency;
(iv) generating a normalized codon usage table by normalising the usage frequencies of the codons not removed in step (iii); and
(v) generating an optimized nucleotide sequence encoding the amino acid sequence by selecting a codon for each amino acid in the amino acid sequence based on the usage frequency of the one or more codons associated with the amino acid in the normalized codon usage table.

2. The method according to claim 1, wherein normalising comprises:
(a) distributing the usage frequency of each codon associated with a first amino acid and removed in step (iii) to the remaining codons associated with the first amino acid; and
(b) repeating step (a) for each amino acid to produce the normalized codon usage table.

3. The method according to claim 2, wherein the usage frequency of the removed codons is distributed equally amongst the remaining codons.

4. The method according to claim 2, wherein the usage frequency of the removed codons is distributed amongst the remaining codons proportionally based on the usage frequency of each remaining codon.

5. The method according to any preceding claim, wherein selecting a codon for each amino acid comprises:
(a) identifying, in the normalized codon usage table, the one or more codons associated with a first amino acid of the amino acid sequence;
(b) selecting a codon associated with the first amino acid, wherein the probability of selecting a certain codon is equal to the usage frequency associated with the codon associated with the first amino acid in the normalized codon usage table; and
(c) repeating steps (a) and (b) until a codon has been selected for each amino acid in the amino acid sequence.

6. The method according to any preceding claim, wherein step (v) is performed a plurality of times to generate a list of optimized nucleotide sequences.

7. The method according to any preceding claim, wherein the threshold frequency is selectable by a user.

8. The method according to any preceding claim, wherein the threshold frequency is in the range of 5% - 30%, in particular 5%, 10%, or 15%, or 20%, or 25%, or 30%, or, in particular, 10%.

9. The method according to any one of claims 6 to 8, further comprising:
screening the list of optimized nucleotide sequences to identify and remove optimized nucleotide sequences failing to meet one or more criteria.

10. The method according to claim 9, wherein screening the list of optimized nucleotide sequences comprises, for each of the one or more criteria:
determining whether each optimized nucleotide sequence in the list, or most recently updated list, of optimized nucleotide sequences meets the criterion; and
updating the list of optimized nucleotide sequences by removing any nucleotide sequence from the list, or most recently updated list, if the nucleotide sequence does not meet the criterion.

11. The method according to claim 10, wherein determining whether each optimized nucleotide sequence in the list, or most recently updated list, of optimized nucleotide sequences meets the criterion comprises, for each nucleotide sequence:
determining whether a first portion of the nucleotide sequence meets the criterion, and wherein updating the list of optimized nucleotide sequences comprises:
removing the nucleotide sequence if the first portion does not meet the criterion.

12. The method according to claim 11, wherein determining whether each optimized nucleotide sequence in the list, or most recently updated list, of optimized nucleotide sequences meets the criterion further comprises, for each nucleotide sequence:
determining whether one or more additional portions of the nucleotide sequence meets the criterion, wherein the additional portions are non-overlapping with each other and with the first portion, and wherein updating the list of optimized sequences comprises:
removing the nucleotide sequence if any portion does not meet the criterion, optionally wherein determining whether an optimized nucleotide sequence meets the criterion is halted when any portion is determined not to meet the criterion.

13. The method according to claim 11 or 12, wherein the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides, e.g., 30 nucleotides, e.g., 100 nucleotides.

14. The method according to any one of claims 9 to 13, wherein a first criterion comprises the nucleotide sequence not containing a termination signal, such that determining and updating comprise:
determining whether each optimized nucleotide sequence in the list, or most recently updated list, of optimized nucleotide sequences contains a termination signal; and
updating the list of optimized nucleotide sequences by removing any nucleotide sequence from the list, or most recently updated list, if the nucleotide sequence contains one or more termination signals.

15. The method according to claim 14, wherein the one or more termination signals has/have the following nucleotide sequence:
5′-X₁ATCTX₂TX₃-3′,
wherein X₁, X₂ and X₃ are independently selected from A, C, T or G.

16. The method according to claim 15, wherein the one or more termination signal has/have one or more of the following nucleotide sequences:
TATCTGTT; and/or
TTTTTT; and/or
AAGCTT; and/or
GAAGAGC; and/or
TCTAGA.

17. The method according to claim 16, wherein the one or more termination signals has/have the following nucleotide sequence:
5′-X₁AUCUX₂UX₃-3′,
wherein X₁, X₂ and X₃ are independently selected from A, C, U or G.

18. The method according to claim 17, wherein the one or more termination signals has/have one of the following nucleotide sequences:
UAUCUGUU; and/or
UUUUUU; and/or
AAGCUU; and/or
GAAGAGC; and/or
UCUAGA.

19. The method according to any one of claims 9 to 18, wherein a second criterion comprises the nucleotide sequence having a guanine-cytosine content within a predetermined guanine-cytosine content range, such that determining and updating comprise:
determining the guanine-cytosine content of each of the optimized nucleotide sequences in the list, or most recently updated list, of optimized nucleotide sequences, wherein the guanine-cytosine content of a sequence is the percentage of bases in the nucleotide sequence that are guanine or cytosine;
updating the list of optimized nucleotide sequences by removing any nucleotide sequence from the list, or most recently updated list, if its guanine-cytosine content falls outside the predetermined guanine-cytosine content range.

20. The method according to claim 19, wherein the predetermined guanine-cytosine content range is selectable by a user.

21. The method according to claim 19 or 20, wherein the predetermined guanine-cytosine content range is 15% - 75%, or 40% - 60%, or, in particular 30% - 70%.

22. The method according to any one of claims 9 to 21, wherein a third criterion comprises the nucleotide sequence having a codon adaptation index greater than a predetermined codon adaptation index threshold, such that determining and updating comprise:
determining the codon adaptation index of each of the optimized nucleotide sequences in the list, or most recently updated list, of optimized nucleotide sequences, wherein the codon adaptation index of a sequence is a measure of codon usage bias and can be a value between 0 and 1;
updating the list, or most recently updated list, of optimized nucleotide sequences by removing any nucleotide sequence if its codon adaptation index is less than or equal to the predetermined codon adaptation index threshold.

23. The method according to claim 22, wherein the codon adaptation index threshold is selectable by a user.

24. The method according to claim 22 or 23, wherein the codon adaptation index threshold is 0.7, or 0.75, or 0.85, or 0.9, or, in particular, 0.8.

25. The method according to any one of claims 9 to 24, wherein a fourth criterion comprises the nucleotide sequence not containing at least 2, for example 3, adjacent identical codons, such that determining and updating comprise:
determining whether any optimized nucleotide sequence in the list, or most recently updated list, of optimized nucleotide sequences, containing at least 2, for example 3 or more, adjacent identical codons; and
updating the list, or most recently updated list, of optimized nucleotide sequences by removing any nucleotide sequence if it contains at least 2, for example 3 or more, adjacent identical codons.

26. The method according to claim 25, wherein the fourth criterion is applied only in respect of codons whose frequency in the normalized codon usage table is less than an adjacency rarity threshold, wherein the adjacency rarity threshold is between 10 and 50%, for example between 15 and 40 %, for example between 20 and 30%.

27. The method according to any preceding claim, wherein the amino acid sequence is received from a database of amino acid sequences.

28. The method according to claim 26, further comprising requesting the amino acid sequence from the database of amino acid sequences, wherein the amino acid sequence is received in response to the request.

29. The method according to any precedingclaim, wherein the first codon usage table is received from a database of codon usage tables.

30. The method according to claim 29, further comprising requesting the first codon usage table from the database of codon usage tables, wherein the first codon usage table is received in response to the request.

31. The method according to any preceding claim, further comprising displaying at least one optimized nucleotide sequence on a screen.

32. A computer program comprising instructions which, when the program is executed by a computer, cause the computer to carry out the method of any preceding claim.

33. A data processing system comprising means for carrying out the method of any preceding claim.

34. A computer-readable data carrier having stored thereon the computer program of claim 32.

35. A data carrier signal carrying the computer program of claim 32.

36. A method for synthesizing a nucleotide sequence, comprising:
performing the computer-implemented method of any one of claims 1 to 31 to generate at least one optimized nucleotide sequence; and
synthesizing at least one of the generated optimized nucleotide sequences.

37. The method according to claim 36, wherein the method further comprises inserting the synthesized optimized sequence in a nucleic acid vector for use in vitro transcription.

38. The method according to claim 36 or 37, wherein the method further comprises inserting one or more termination signals at the 3′ end of the synthesized optimized nucleotide sequence.

39. The method according to claim 38, wherein the one or more termination signals are encoded by the following nucleotide sequence:
5′-X₁ATCTX₂TX₃-3′,
wherein X₁, X₂ and X₃ are independently selected from A, C, T or G.

40. The method according to claim 38 or 39, wherein the one or more termination signals are encoded by one or more of the following nucleotide sequences:
TATCTGTT;
TTTTTT;
AAGCTT;
GAAGAGC; and/or
TCTAGA.

41. The method according to any one of claims 38 to 40, wherein more than one termination signal is inserted, and said termination signals are separated by 10 base pairs or fewer, e.g. separated by 5-10 base pairs.

42. The method according to claim 40, wherein the more than one termination signals are encoded by the following nucleotide sequence: (a) 5′-X₁ATCTX₂TX₃-(Z_{N})-X₄ATCTX₅TX₆-3′ or (b) 5′-X₁ATCTX₂TX₃-(Z_{N})- X₄ATCTX₅TX₆-(Z_{M})- X₇ATCTX₈TX₉-3′, wherein X₁, X₂, X₃, X₄, X₅, X₆, X₇, X₈ and X₉ are independently selected from A, C, T or G, Z_{N} represents a spacer sequence of N nucleotides, and Z_{M} represents a spacer sequence of M nucleotides, each of which are independently selected from A, C, T of G, and wherein N and/or M are independently 10 or fewer.

43. The method according to any one of claims 37 to 42, wherein the nucleic acid vector comprises an RNA polymerase promoter operably linked to the optimized nucleotide sequence, optionally wherein the RNA polymerase promoter is a SP6 RNA polymerase promoter or a T7 RNA polymerase promoter.

44. The method according to any one of claims 37 to 43, wherein the nucleic acid vector comprises a nucleotide sequence encoding a 5′ UTR operably linked to the optimized nucleotide sequence.

45. The method according to claim 44, wherein the 5′ UTR is different to the 5′ UTR of a naturally occurring mRNA encoding the amino acid sequence.

46. The method according to claim 42, wherein the 5′ UTR has the nucleotide sequence of SEQ ID NO: 16.

47. The method according to any one of claims 37 to 46, wherein the nucleic acid vector comprises a nucleotide sequence encoding a 3′ UTR operably linked to the optimized nucleotide sequence.

48. The method according to claim 46, wherein the 3′ UTR is different to the 3′ UTR of a naturally occurring mRNA encoding the amino acid sequence.

49. The method according to claim 48, wherein the 3′ UTR has the nucleotide sequence of SEQ ID NO: 17 or SEQ ID NO: 18.

50. The method according to any one of claims 37 to 49 wherein the nucleic acid vector is a plasmid.

51. The method according to claim 50, wherein the plasmid is linearized before in vitro transcription.

52. The method according to claim 50, wherein the plasmid is not linearized before in vitro transcription.

53. The method according to claim 52, wherein the plasmid is supercoiled.

54. The method according to any one of claims 36 to 53, wherein the method further comprises using at least one of the synthesized optimized nucleotide sequences in in vitro transcription to synthesize mRNA.

55. The method according to claim 54, wherein the mRNA is synthesized by a SP6 RNA polymerase.

56. The method according to claim 55, wherein the SP6 RNA polymerase is a naturally occurring SP6 RNA polymerase.

57. The method according to claim 55, wherein the SP6 RNA polymerase is a recombinant SP6 RNA polymerase.

58. The method according to claim 57, wherein the SP6 RNA polymerase comprises a tag.

59. The method according to claim 58, wherein the tag is a his-tag.

60. The method according to claim 54, wherein the mRNA is synthesized by a T7 RNA polymerase.

61. The method according to any one of claims 54 to 60, wherein the method further comprises a separate step of capping and/or tailing the synthesized mRNA.

62. The method according to any one of claims 54 to 60, wherein capping and tailing occurs during in vitro transcription.

63. The method according to any one of claims 54 to 62, wherein the mRNA is synthesized in a reaction mixture comprising NTPs at a concentration ranging from 1-10 mM each NTP, the DNA template at a concentration ranging from 0.01-0.5 mg/ml, and the SP6 RNA polymerase at a concentration ranging from 0.01-0.1 mg/ml.

64. The method according to claim 63, wherein the reaction mixture comprises NTPs at a concentration of 5 mM each NTP, the DNA template at a concentration of 0.1 mg/ml, and the SP6 RNA polymerase at a concentration of 0.05 mg/ml.

65. The method according to any one of claims 54 to 64, wherein the mRNA is synthesized at a temperature ranging from 37-56° C.

66. The method according to any one of claims 63 to 65, wherein the NTPs are naturally-occurring NTPs.

67. The method according to any one of claims 63 to 65, wherein the NTPs comprise modified NTPs.

68. The method according to any one of claims 36 to 67, wherein the method further comprises transfecting the synthesized optimized nucleotide sequence into a cell either in vitro or in vivo.

69. The method according to claim 68, wherein the expression level of the protein encoded by the synthesized optimized nucleotide sequence in transfected cell is determined.

70. The method according to claim 68 or 69, wherein the functional activity of the protein encoded by the synthesized optimized nucleotide sequence is determined.

71. The method according to any one of claims 1 to 31, further comprising synthesizing a reference nucleotide sequence encoding the amino acid sequence and the at least one optimized nucleotide sequence according to the method of any one of claims 36 to 70, and contacting the reference nucleotide sequence and the at least one optimized nucleotide sequence with a separate cell or organism, wherein the cell or organism contacted with the at least one synthesized optimized nucleotide sequence produces an increased yield of the protein encoded by the optimized nucleotide sequence compared to the yield of the protein encoded by the reference nucleotide sequence produced by the cell or organism contacted with the synthesized reference nucleotide sequence.

72. The method of any one of claims 36 to 70, wherein the method further comprises producing a therapeutic composition comprising an mRNA encoding a therapeutic peptide, polypeptide, or protein for use in the delivery to or treatment of a subject.

73. The method of claim 72, wherein the mRNA encodes cystic fibrosis transmembrane conductance regulator (CFTR) protein.

74. The method according to any one of claims 1 to 31, wherein the at least one optimized nucleotide sequence, when synthesized, is configured to increase the expression of the protein encoded by the at least one optimized nucleotide sequence compared to the expression of the protein encoded by the reference nucleotide sequence, when synthesized.

75. The method of any one of claims 71 to 74, wherein the reference nucleotide sequence is (a) a naturally occurring nucleotide sequence encoding the amino acid sequence or (b) a nucleotide sequence encoding the amino acid sequence generated by a method other than the method according to any one of claims 1 to 31.

76. A synthesized optimized nucleotide sequence generated according to the methods of any one of claims 36 to 67 and 72 to 75 for use in therapy.

77. A method of treatment comprising administering the synthesized optimized nucleotide sequence generated according to the method of any one of claims 36 to 67 and 72 to 75 to a human subject in need of such treatment.

78. An in vitro synthesized nucleic acid comprising an optimized nucleotide sequence consisting of codons associated with a usage frequency which is greater than or equal to 10%; wherein the optimized nucleotide sequence:
(iv) does not contain a termination signal having one of the following nucleotide sequences:
5′-X₁AUCUX₂UX₃-3′, wherein X₁, X₂ and X₃ are independently selected from A, C, U or G; and 5′-X₁AUCUX₂UX₃-3′, wherein X₁, X₂ and X₃ are independently selected from A, C, U or G;
(v) does not contain any negative cis-regulatory elements and negative repeat elements; and
(vi) has a codon adaptation index greater than 0.8;
wherein, when divided into non-overlapping 30 nucleotide-long portions, each portion of the optimized nucleotide sequence has a guanine cytosine content range of 30% - 70%.

79. The in vitro synthesized nucleic acid of claim 77, wherein the optimized nucleotide sequence does not contain a termination signal having one of the following sequences:
TATCTGTT; TTTTTT; AAGCTT; GAAGAGC; TCTAGA; UAUCUGUU; UUUUUU; AAGCUU; GAAGAGC; UCUAGA.

80. The in vitro synthesized nucleic acid of claim 78 or 79, wherein the nucleic acid is mRNA.

81. The in vitro synthesized nucleic acid of any one of claims 78 to 80 for use in therapy.


SEQUENCE LISTING (32 sequences)

SEQ ID NO: 1 - protein, 874 aa, Bacteriophage SP6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 2 - DNA, 2625 bp, Bacteriophage SP6
atgcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagtgcgctggatggttgatacaggtatcgaagtacctgagcaaggggagttcgaccttaacgaaatcatggattctgaatacgtatttgcctaa

SEQ ID NO: 3 - DNA, 18 bp, Bacteriophage SP6
atttaggtgacactatag

SEQ ID NO: 4 - DNA, 23 bp, Artificial Sequence
  Synthetic oligonucleotide
atttaggggacactatagaagag

SEQ ID NO: 5 - DNA, 22 bp, Artificial Sequence
  Synthetic oligonucleotide
atttaggggacactatagaagg

SEQ ID NO: 6 - DNA, 23 bp, Artificial Sequence
  Synthetic oligonucleotide
atttaggggacactatagaaggg

SEQ ID NO: 7 - DNA, 20 bp, Artificial Sequence
  Synthetic oligonucleotide
atttaggtgacactatagaa

SEQ ID NO: 8 - DNA, 22 bp, Artificial Sequence
  Synthetic oligonucleotide
atttaggtgacactatagaaga

SEQ ID NO: 9 - DNA, 23 bp, Artificial Sequence
  Synthetic oligonucleotide
atttaggtgacactatagaagag

SEQ ID NO: 10 - DNA, 22 bp, Artificial Sequence
  Synthetic oligonucleotide
atttaggtgacactatagaagg

SEQ ID NO: 11 - DNA, 23 bp, Artificial Sequence
  Synthetic oligonucleotide
atttaggtgacactatagaaggg

SEQ ID NO: 12 - DNA, 23 bp, Artificial Sequence
  Synthetic oligonucleotide
  misc_feature (22): n is a, c, t or g
atttaggtgacactatagaagng

SEQ ID NO: 13 - DNA, 24 bp, Artificial Sequence
  Synthetic oligonucleotide
catacgatttaggtgacactatag

SEQ ID NO: 14 - DNA, 18 bp, Artificial Sequence
  Bacteriophage T7
taatacgactcactatag

SEQ ID NO: 15 - protein, 1480 aa, Artificial Sequence
  Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 16 - RNA, 140 nt, Artificial Sequence
  5' UTR sequence
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 17 - RNA, 105 nt, Artificial Sequence
  3' UTR sequence
cggguggcaucccugugaccccuccccagugccucuccuggcccuggaaguugccacuccagugcccaccagccuuguccuaauaaaauuaaguugcaucaagcu

SEQ ID NO: 18 - RNA, 105 nt, Artificial Sequence
  3' UTR sequence
ggguggcaucccugugaccccuccccagugccucuccuggcccuggaaguugccacuccagugcccaccagccuuguccuaauaaaauuaaguugcaucaaagcu

SEQ ID NO: 19 - DNA, 582 bp, Artificial Sequence
  Homo sapiens EPO sequence, codon optimized, reference
atgggtgtgcacgaatgtcctgcttggctgtggctccttctctccctgctgtccctgcctcttggactcccggtgcttggagcacccccgagactgatctgcgacagcagggtgctcgagcgctacctcctggaagccaaggaagccgaaaacatcactactggctgcgccgaacactgctccctgaacgagaacatcaccgtgccggacaccaaggtcaacttctacgcgtggaagagaatggaggtcggacagcaagccgtggaagtgtggcagggacttgcgctcctgtcggaagccgtgctgaggggacaagccctgctcgtgaacagctcacagccttgggagcccctgcagctgcatgtcgacaaggccgtgtccggactgcgctcactgaccactctgctgagggccttgggtgcccagaaagaggctatttccccaccggatgcagcctcggcagctcctctgcggaccattacggcggacacctttcggaagctgttccgcgtctacagcaatttcctccgggggaagttgaaactgtataccggcgaagcctgtcggactggcgatcgctga

SEQ ID NO: 20 - DNA, 582 bp, Artificial Sequence
  Homo sapiens EPO sequence, codon optimized, #1
atgggggttcatgagtgcccagcttggctttggctcctgctcagcttgcttagtctccctttgggcctgcccgtgctgggcgcccctccacgcttgatctgtgacagcagggtcttggaacggtatttgcttgaagctaaagaagctgagaacataacaacgggatgtgctgaacattgctccttgaacgaaaacatcacagttcccgacacaaaagtcaatttttacgcatggaagcggatggaggttggccagcaagctgtggaggtctggcaagggctggctcttctcagtgaagccgtgctgcgcggacaagcactcttggtgaactccagccagccctgggagccccttcagctccatgtcgataaagcagttagcggcctccgatcattgactaccctccttagggctttgggtgcacaaaaagaggccatttcaccaccggacgcggcaagtgctgctccgttgcgaactataactgctgacaccttccggaaactttttcgggtatattccaactttctcagggggaaactcaagctctacaccggcgaggcgtgccgaactggagaccgctga

SEQ ID NO: 21 - DNA, 582 bp, Artificial Sequence
  Homo sapiens EPO sequence, codon optimized, #2
atgggcgtacatgaatgcccggcatggctttggctgctgctgtccctgctgagtttgccgctgggcctccccgtcctcggcgctcccccgagactcatttgcgactctagggtcctcgaacgctatctgctggaagcaaaagaagctgagaacataactacaggatgcgctgagcactgttccttgaatgagaatatcacagtacctgacactaaggtgaatttttacgcatggaaacgcatggaagtgggtcagcaggccgtggaagtgtggcagggcctggcgctgctgtccgaggctgttcttagaggccaagccttgttggtcaattcctctcaaccctgggagcccctccagctgcatgttgataaagccgtctctggtctccggtcccttaccaccctgctcagggcacttggcgcacagaaggaagctatctcccccccagacgctgccagtgccgcccccctccggactattaccgccgatactttcaggaaactgtttcgagtctatagcaattttctccgcgggaaactgaagctgtatacaggtgaggcctgcaggacaggagatcgctga

SEQ ID NO: 22 - DNA, 582 bp, Artificial Sequence
  Homo sapiens EPO sequence, codon optimized, #3
atgggcgtgcacgaatgtcctgcttggctgtggctgctgctgagtctgctgtctctgcctctgggactgcctgttcttggagcccctcctagactgatctgcgacagcagagtgctggaaagatacctgctggaagccaaagaggccgagaacatcacaacaggctgtgccgagcactgcagcctgaacgagaatatcaccgtgcctgacaccaaagtgaacttctacgcctggaagcggatggaagtgggacagcaggctgtggaagtttggcaaggactggccctgctgtctgaagctgttctgagaggacaggctctgctggtcaatagctctcagccttgggaacctctccagctgcatgtggataaggccgtgtctggcctgagaagcctgacaacactgctgagagccctgggagcccagaaagaggccatttctccacctgatgctgccagcgctgcccctctgagaacaatcaccgccgacaccttcagaaagctgttccgggtgtacagcaacttcctgcggggcaagctgaaactgtacaccggcgaagcctgcagaaccggcgatagataa

SEQ ID NO: 23 - DNA, 582 bp, Artificial Sequence
  Homo sapiens EPO sequence, codon optimized, #4
atgggggtgcacgagtgccctgcctggctgtggttgctgctgtccctgctgtctctgccactgggactgccagtgctgggagctccacctaggctgatctgcgacagccgggtcctggagaggtacctgctcgaggccaaggaggccgagaacattaccacaggctgcgccgagcactgcagcctgaacgagaacattacagtgcccgatacaaaggtgaacttctacgcctggaagaggatggaggtgggccagcaggccgtggaggtgtggcaggggctggccctgctgagcgaggccgtgctgaggggccaagccctgctggtcaacagcagccagccttgggagcccctgcagctccacgtggacaaggctgtgtctggcttgaggtctctcacaacattgctgagggccctgggcgcacagaaagaagctatcagcccacctgatgccgctagtgccgctccactgcggacaattaccgccgatacctttagaaaattgttcagggtctactccaactttttgcgcgggaagctgaagctctataccggcgaggcctgccggacaggggacagatga

SEQ ID NO: 24 - DNA, 582 bp, Artificial Sequence
  Homo sapiens EPO sequence, codon optimized, #5
atgggagtgcacgaatgtcctgcatggctctggctcctgctgtctctcctgagcctgccactgggactcccagtgctgggagcaccccctaggctgatctgcgattctcgggtgctggagcgctacctgctcgaggctaaggaggccgagaatatcactactgggtgtgccgaacactgtagcctcaatgaaaacattacagtcccagataccaaggtgaacttttatgcatggaagaggatggaggtcgggcagcaggcagtggaggtgtggcagggactggctctgctgtccgaagccgtgctcagaggtcaggccctgctggttaattccagccagccttgggaacctctgcagctgcatgtggacaaggcagtgtctggcctgagatcccttactacactgctgagagcactgggggctcagaaagaagctatttccccaccagacgccgcctcagcagcacctctccggaccatcactgctgacaccttccgcaagctctttagggtgtactccaacttcctgcgcgggaagctcaagctgtacaccggcgaagcctgcaggaccggggatcgctga

SEQ ID NO: 25 - DNA, 4443 bp, Artificial Sequence
  Homo sapiens CFTR sequence, codon optimized, reference
atgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaactcgatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctgtccctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtgtccctggcacctcaagcgaatcttaccgagctcgacatctactcccggagactgtcgcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaa

SEQ ID NO: 26 - DNA, 4443 bp, Artificial Sequence
  Homo sapiens CFTR sequence, codon optimized, hCFTR #1
atgcagcggtccccgctcgaaaaggccagtgtcgtgtccaaactcttcttctcatggactcggcctatccttagaaaggggtatcggcagaggcttgagttgtctgacatctaccagatcccctcggtagattcggcggataacctctcggagaagctcgaacgggaatgggaccgcgaactcgcgtctaagaaaaacccgaagctcatcaacgcactgagaaggtgcttcttctggcggttcatgttctacggtatcttcttgtatctcggggaggtcacaaaagcagtccaacccctgttgttgggtcgcattatcgcctcgtacgaccccgataacaaagaagaacggagcatcgcgatctacctcgggatcggactgtgtttgcttttcatcgtcagaacacttttgttgcatccagcaatcttcggcctccatcacatcggtatgcagatgcgaatcgctatgtttagcttgatctacaaaaagacactgaaactctcgtcgcgggtgttggataagatttccatcggtcagttggtgtccctgcttagtaataacctcaacaaattcgatgagggactggcgctggcacatttcgtgtggattgccccgttgcaagtcgcccttttgatgggccttatttgggaactcttgcaggcatctgccttttgtggcctgggatttctgattgtgttggcattgtttcaggctgggcttgggcggatgatgatgaagtatcgcgaccagagagcgggtaaaatctcggaaagactcgtcatcacttcggaaatgatcgaaaacatccagtcggtcaaagcctattgctgggaagaagctatggagaagatgattgaaaacctccgccaaactgagctgaaactgacccgcaaggcggcgtatgtccggtatttcaattcgtcagcgttcttcttttccgggttcttcgttgtctttctctcggttttgccttatgccttgattaaggggattatcctccgcaagattttcaccacgatttcgttctgcattgtattgcgcatggcagtgacacggcaatttccgtgggccgtgcagacatggtatgactcgcttggagcgatcaacaaaatccaagacttcttgcaaaagcaagagtacaagaccctggagtacaatcttactactacggaggtagtaatggagaatgtgacggctttttgggaagagggttttggagagctcttcgagaaagcaaagcagaataacaacaaccgcaagacctcaaatggggacgattccctgtttttctcgaacttctccctgctcggaacacccgtgttgaaggacatcaatttcaagattgagaggggacagcttctcgcggtagcgggaagcactggtgcgggaaaaactagcctcttgatggtgattatgggggagcttgagcccagcgaggggaagattaaacactccgggcgtatctcattctgtagccagttttcatggatcatgcccggaaccattaaagagaacatcattttcggagtatcctatgatgagtaccgatacagatcggtcattaaggcgtgccagttggaagaggacatttctaagttcgccgagaaggataacatcgtcttgggagaagggggtattacattgtcgggagggcagcgagcgcggatcagcctcgcgagagcggtatacaaagatgcagatttgtacctgctcgattcaccgtttggatacctcgacgtattgacagaaaaagaaatcttcgagtcgtgcgtgtgtaaacttatggctaataagacgagaatcctggtgacatcaaaaatggaacaccttaagaaggcggacaagatcctgatcctccacgaaggatcgtcctacttttacggcactttctcagagttgcaaaacttgcagccggacttctcaagcaaactcatggggtgtgactcattcgaccagttcagcgcggaacggcggaactcgatcttgacggaaacgctgcaccgattctcgcttgagggtgatgccccggtatcgtggaccgagacaaagaagcagtcgtttaagcagacaggagaatttggtgagaaaagaaagaacagtatcttgaatcctattaactcaattcgcaagttctcaatcgtccagaaaactccactgcagatgaatggaattgaagaggattcggacgaacccctggagcgcaggcttagcctcgtgccggattcagagcaaggggaggccattcttccccggatttcggtgatttcaaccggacctacacttcaggcgaggcgaaggcaatccgtgctcaacctcatgacgcattcggtaaaccaggggcaaaacattcaccgcaaaacgacggcctcaacgagaaaagtgtcacttgcaccccaggcgaatttgactgaactcgacatctacagccgtaggctttcgcaagaaaccggacttgagatcagcgaagaaatcaatgaagaagatttgaaagagtgtttctttgatgacatggaatcaatcccagcggtgacaacgtggaacacatacttgcgttacatcacggtgcacaagtccttgattttcgtcctcatttggtgcctcgtgatctttctcgctgaggtcgcagcgtcacttgtggtcctctggctgcttggtaatacgcccttgcaagacaaaggcaattctacacactcaagaaacaattcctatgccgtgattatcacttctacaagctcgtattacgtgttttacatctacgtaggagtggccgacactctgctcgcgatgggtttcttccgaggactcccactcgttcacacgcttatcactgtctccaagattctccaccataagatgcttcatagcgtactgcaggctcccatgtccaccttgaatacgctcaaggcgggaggtattttgaatcgcttctcaaaagatattgcaattttggatgaccttctgcccctgacgatcttcgacttcatccagttgttgctgatcgtgattggggctattgcagtagtcgctgtcctccagccttacatttttgtcgcgaccgttccggtgatcgtggcgtttatcatgctgcgggcctatttcttgcagacgtcacagcagcttaagcaactggagtctgaagggaggtcgcctatctttacgcatcttgtgaccagtttgaagggattgtggacgttgcgcgcctttggcaggcagccctactttgaaacactgttccacaaagcgctgaatctccatacggcaaattggtttttgtatttgagtaccctccgatggtttcagatgcgcattgagatgatttttgtgatcttctttatcgcggtgacttttatctccatcttgaccacgggagagggcgagggacgggtcggtattatcctgacactcgccatgaacattatgagcactttgcagtgggcagtgaacagctcgattgatgtggatagcctgatgaggtccgtttcgagggtctttaagttcatcgacatgccgacggagggaaagcccacaaaaagtacgaaaccctataagaatgggcaattgagtaaggtaatgatcatcgagaacagtcacgtgaagaaggatgacatctggcctagcgggggtcagatgaccgtgaaggacctgacggcaaaatacaccgagggagggaacgcaatccttgaaaacatctcgttcagcattagccccggtcagcgtgtggggttgctcgggaggaccgggtcaggaaaatcgacgttgctgtcggccttcttgagacttctgaatacagagggtgagatccagatcgacggcgtttcgtgggatagcatcaccttgcagcagtggcggaaagcgtttggagtaatcccccaaaaggtctttatctttagcggaaccttccgaaagaatctcgatccttatgaacagtggtcagatcaagagatttggaaagtcgcggacgaggttggccttcggagtgtaatcgagcagtttccgggaaaactcgactttgtccttgtagatgggggatgcgtcctgtcgcatgggcacaagcagctcatgtgcctggcgcgatccgtcctctctaaagcgaaaattcttctcttggatgaaccttcggcccatctggacccggtaacgtatcagatcatcagaaggacacttaagcaggcgtttgccgactgcacggtgattctctgtgagcatcgtatcgaggccatgctcgaatgccagcaatttcttgtcatcgaagagaataaggtccgccagtacgactccatccagaagctgcttaatgagagatcattgttccggcaggcgatttcaccatccgatagggtgaaactttttccacacagaaattcgtcgaagtgcaagtccaaaccgcagatcgcggccttgaaagaagagactgaagaagaagttcaagacacgcgtctttaa

SEQ ID NO: 27 - DNA, 4443 bp, Artificial Sequence
  Homo sapiens CFTR sequence, codon optimized, hCFTR #2
atgcagcgttctcccctggagaaggcttctgtggtgagtaaactttttttctcctggaccagacctatcctgaggaaaggctacaggcagagactggagctctctgacatataccagataccttcagtcgatagcgccgacaacctgagcgagaagctggaacgcgagtgggacagagagctggcaagcaagaagaacccaaagctgattaatgccctgagaaggtgtttcttctggagattcatgttctacggaatctttctgtatctgggggaggttacaaaggctgtgcaacccctgctgctcggcagaatcatcgcctcatacgatccagacaacaaggaagaaagaagcatcgccatctacctgggcattggcctctgcctcctgtttattgtgcggactctgctgctgcacccagcaattttcgggttgcatcatattggcatgcagatgcgcattgctatgttttccctcatctacaaaaagacactgaaactcagctcccgggtgctggacaagatctccatcggccaactggtgtctctcctgagcaataacttgaataagttcgacgaagggctggccctggcacacttcgtgtggattgcccccctgcaggtggccctgctgatgggactgatttgggaactgctgcaggctagcgctttctgcggcctggggttcctgatcgtgctggcactgtttcaggcaggcctgggccgtatgatgatgaagtacagagaccagagggccgggaagatctccgaacggctcgttattacctctgagatgatcgagaacattcagtctgtgaaagcctactgctgggaggaggctatggagaagatgatcgagaatctgagacagaccgagctgaagctgaccagaaaggccgcctacgtgaggtacttcaacagcagtgccttcttcttctctgggttcttcgttgtgtttctgagcgtgctgccatacgctctcatcaaaggcatcatcctgcggaagatcttcaccaccatcagcttttgcatcgtgcttagaatggccgtgacacggcagttcccatgggccgttcaaacttggtatgattccctgggcgccatcaacaaaatccaggatttcctgcagaagcaggaatacaagacactcgaatataacctcacaactactgaggtggttatggagaacgtgactgccttctgggaggaggggttcggagagctttttgagaaggccaaacagaataataataaccgcaaaaccagcaacggcgacgacagcctgttcttctccaatttttctctcctgggaacacccgtcctcaaagacatcaactttaagatcgagaggggccagctgctcgccgtcgccggatccacaggcgccggcaagacctctctgctgatggttatcatgggcgaactggagccctccgagggcaagattaagcactcaggaagaatctccttttgtagccagttcagttggattatgcccggcactattaaggagaatatcatttttggggtgagctatgatgagtatcggtatcggagcgttatcaaagcctgtcagctggaggaggatatcagcaagttcgcagagaaggataatattgtgctgggagagggaggaatcaccctgagcggaggccagagagccagaatctcactggcccgggccgtctacaaggacgccgacctttaccttctggacagtccctttggatatctggatgtgctgactgaaaaggagatcttcgagtcttgtgtgtgcaagctgatggctaacaagacccggatcctagtgactagtaagatggagcacctgaagaaggcagacaagatcttgattctgcacgagggatcctcttacttttacggcacctttagcgagctgcagaacctccagcccgatttctcatctaagctgatgggctgtgatagcttcgaccagttctctgccgagcgcagaaacagcatcctgacagagacactgcaccggttttcactggagggcgacgcccctgtcagctggaccgagaccaaaaagcagtctttcaagcagacaggcgagttcggcgagaagcgcaaaaacagcatcctgaatccaatcaactctataaggaagtttagcatcgtgcagaagacacccctccagatgaacggcatcgaagaggacagtgacgagcccctggagcggcgcctgagcctcgtgcctgacagcgaacagggcgaggccatcctgcctaggatcagcgtgatttcaaccgggccaacactgcaggctaggagaagacagtcagtgcttaacctgatgacacatagcgtgaatcagggacagaacatccatcgaaaaaccacagcctctactcgcaaagtgtcactggctcctcaggctaatctgacagagctggacatctatagcaggaggctgagccaggagacaggcctggagatcagtgaggagatcaacgaagaggacctgaaggagtgctttttcgatgacatggagagtatccccgccgtcaccacctggaatacctacctccggtacatcacagtgcacaagtccctcatctttgtgctgatttggtgcctcgtgatctttctcgcagaagtggccgcctccctggtggtgctgtggctgttggggaatactccactgcaggacaaaggcaattctacacacagcaggaataattcctatgccgtgattatcaccagcacatcctcttactacgtgttctacatctacgtgggagtggcagatactctgcttgcaatgggcttcttcagggggctgcccctggtgcacacactgatcacagtgtccaagatcctccaccataaaatgctccacagcgtgctgcaggcacccatgagcaccctgaacacactgaaggccggcggcatcctgaatcgcttttccaaagacatcgccatcctcgacgatctcctgccactgaccatcttcgattttatccagctgctgctgatcgtgatcggggccatcgccgtggtggccgtgctgcagccatacattttcgtggctacagtgcccgtgatcgttgcctttatcatgctgagagcctacttcctgcagacttctcagcagctgaagcagctggagagcgaagggagaagccccatcttcactcacctggtgacaagcctgaagggactctggaccctgagagccttcggccggcagccctatttcgagaccctgtttcacaaggccctcaacctgcacacagccaactggttcctctacctgtccaccctgaggtggttccagatgaggattgaaatgatcttcgtgatttttttcatcgccgtgacattcattagcattctgaccaccggcgagggggaggggagagtgggcatcatcctgacccttgccatgaacattatgagcacactgcagtgggccgtgaatagtagtatcgacgtggacagtctgatgaggtccgtgagccgggtgttcaagttcattgacatgcccacagaagggaaacccaccaaaagcaccaagccctacaagaacgggcagctgtccaaggttatgatcatcgagaactctcacgtgaagaaggacgacatttggcccagcggcggccagatgacagtgaaagatctgaccgccaaatacaccgagggaggcaacgccatcctcgaaaacattagcttctctatcagccctggacagagggtgggcctgctgggccggacaggctcagggaagagtactctgctgtcagcattcctgaggctcctgaacacagagggcgagatccagattgacggcgtgtcctgggactccatcaccctgcagcagtggcggaaggctttcggggtgatcccccagaaggtgttcatctttagcggcactttcagaaagaatctggacccttatgagcagtggagtgaccaggagatctggaaagtggccgatgaggtcggactgaggagcgtgatcgagcagtttccagggaagctggactttgtgctggtggatggcggatgcgtgctgtctcacggccataaacagctgatgtgtctggcccggtccgtgctgtctaaggccaagatcctgctgctggacgaaccctccgcccacctggaccccgtgacataccagatcatcaggagaactctcaagcaggccttcgccgactgtaccgtgattctgtgcgagcaccgcattgaagctatgctggagtgtcagcagttcctggtgatcgaggaaaataaggtgaggcagtacgacagcatccagaagctgctgaacgagcgctccctgttccgccaggctatctccccatcagaccgggtgaagctcttcccccacagaaactcctcaaagtgcaagtccaagccccagatcgccgccctgaaggaggagaccgaggaggaggtgcaggacaccaggctgtga

SEQ ID NO: 28 - DNA, 4443 bp, Homo sapiens CFTR sequence, codon optimized, hCFTR #3
atgcagcgctcgcctctggaaaaggcgagcgtcgtgtcaaagctattcttttcttggacccggcccattctcaggaagggctacaggcagaggctggagttgagcgacatctatcagattccttccgtggacagcgccgacaacctgagcgagaagctggaaagggagtgggaccgcgaactggcaagcaaaaagaaccccaagctgatcaatgccctgagaaggtgtttcttttggagattcatgttctacgggatctttctgtatctgggcgaggttacaaaggctgtgcagcccctgctgctcggcagaatcatcgcctcatacgatccagacaacaaggaagaaagaagcatcgccatctacctgggcattggcctctgcctcctgtttattgtgcggactctgctgctgcacccagcaattttcgggttgcatcatattggcatgcagatgcgcattgctatgttttccctcatctacaaaaagacactgaaactcagctcccgggtgctggacaagatctccatcggccaactggtgtctctcctgagcaataacttgaataagttcgacgaagggctggccctggcacacttcgtgtggattgcccccctgcaggtggccctgctgatgggactgatttgggaactgctgcaggctagcgctttctgcggcctggggttcctgatcgtgctggcactgtttcaggcaggcctgggccgtatgatgatgaagtacagagaccagagggccgggaagatctccgaacggctcgttattacctctgagatgatcgagaacattcagtctgtgaaagcctactgctgggaggaggctatggagaagatgatcgagaatctgagacagaccgagctgaagctgaccagaaaggccgcctacgtgaggtacttcaacagcagtgccttcttcttctctggcttcttcgttgtgtttctgagcgtgctgccatacgctctcatcaaaggcatcatcctgcggaagatcttcaccaccatcagcttttgcatcgtgcttagaatggccgtgacccggcagttcccatgggccgtgcaaacttggtatgattccctgggcgccatcaacaaaatccaggatttcctgcagaagcaggaatacaagacactcgaatataatctcacaactactgaggtggttatggagaacgtgactgccttctgggaggaggggttcggagagctttttgagaaggcaaaacagaataacaacaaccgcaaaaccagcaacggcgacgacagcctgttcttctccaatttttctctcctgggaacacccgtcctcaaagacatcaactttaagatcgagaggggacagctgctcgcagtcgccggatccacaggcgccggcaagacctctctgctgatggttatcatgggcgaactggagccatccgagggcaagattaagcacagtggaagaatctccttttgtagccagttcagttggattatgcccggcactattaaggagaatatcatttttggggtgagctatgatgagtatcggtatcggagcgttatcaaagcctgtcagctggaggaggatatcagcaaattcgcagagaaggataatatcgtgctgggggaggggggaatcaccctgagcggaggccagagagccagaatctcactggcccgggccgtctacaaggacgccgacctttaccttctggacagtccctttggatatctggatgtgctgactgaaaaggagatcttcgagtcttgtgtgtgcaagctgatggctaataagacccggatcctagtgaccagtaagatggagcacctgaagaaggcagacaagatcttgattctgcacgagggatcctcttacttttacggcacctttagcgagctgcagaatctccagcccgatttctcatctaagctgatgggctgtgatagcttcgaccagttctctgccgagcgcagaaacagcatcctgacagagacactgcaccggttttcactggagggcgacgcccctgtcagctggaccgagaccaaaaagcagtctttcaagcagacaggcgagttcggcgagaagcgcaaaaacagcatcctgaatccaatcaactctataaggaagtttagcatcgtgcagaagacacccctccagatgaacggcatcgaagaggacagtgacgagcccctggagcggcgcctgagcctcgtgcctgacagcgaacagggcgaggccatcctgcctaggatcagcgtgatttcaaccgggccaacactgcaggctaggagaagacagtcagtgcttaacctgatgacacatagcgtgaatcagggacagaacatccatcgaaaaaccacagcctctactcgcaaagtgtcactggctcctcaggctaatctgacagagctggacatctatagcaggaggctgagccaggagacaggcctggagatcagtgaggagatcaacgaagaggacctgaaggagtgctttttcgatgacatggagagtatccccgccgtcaccacctggaatacctacctccggtacatcacagtgcacaagtccctcatctttgtgctgatttggtgcctcgtgatctttctcgcagaagtggccgcctccctggtggtgctgtggctgttggggaatactccactgcaggacaaaggcaattctacacacagcaggaataattcctatgccgtgattatcaccagcacatcctcttactacgtgttctacatctacgtgggagtggcagatactctgcttgcaatgggcttcttcagggggctgcccctggtgcacacactgatcacagtgtccaagatcctccaccataaaatgctccacagcgtgctgcaggcacccatgagcaccctgaacacactgaaggccggcggcatcctgaatcgcttttccaaagacatcgccatcctcgacgatctcctgccactgaccatcttcgattttatccagctgctgctgatcgtgatcggggccatcgccgtggtggccgtgctgcagccatacattttcgtggctacagtgcccgtgatcgttgcctttatcatgctgagagcctacttcctgcagacttctcagcagctgaagcagctggagagcgaagggagaagccccatcttcactcacctggtgacaagcctgaagggactctggaccctgagagccttcggccggcagccctatttcgagaccctgtttcacaaggccctcaacctgcacacagccaactggtttctctacctgtccaccctgaggtggttccagatgaggattgaaatgatcttcgtgatttttttcatcgccgtgacattcattagcattctgaccaccggcgagggggaggggagagtgggcatcatcctgacccttgccatgaacattatgtccacactgcagtgggccgtgaatagttcaatcgacgtggacagtctgatgaggtccgtgagccgggtgttcaagttcattgacatgcccacagaggggaaacccaccaaaagcaccaagccctacaagaacgggcagctgtccaaggttatgatcatcgagaactctcacgtgaagaaggacgacatttggcccagcggcggccagatgacagtgaaagatctgaccgccaaatacaccgagggaggcaacgccatcctcgaaaacattagcttctctatcagccctggacagagggtgggcctgctgggccggacaggctcagggaagagtactctgctgtcagcattcctgaggctcctgaacacagagggcgagatccagattgacggcgtgtcctgggactccatcaccctgcagcagtggcggaaggctttcggggtgatcccccagaaggtgttcatctttagcggcactttcagaaagaatctggacccttatgagcagtggagtgaccaggagatctggaaagtggccgatgaggtcggactgaggagcgtgatcgagcagtttccagggaagctggactttgtgctggtggatggcggatgcgtgctgtctcacggccataaacagctgatgtgtctggcccggtccgtgctgtctaaggccaagatcctgctgctggacgaaccctccgcccacctggaccccgtgacataccagatcatcaggagaactctcaagcaggccttcgccgactgtaccgtgattctgtgcgagcaccgcattgaagctatgctggagtgtcagcagttcctggtgatcgaggaaaataaggtgaggcagtacgacagcatccagaagctgctgaacgagcgctccctgttccgccaggctatctccccatcagaccgggtgaagctcttcccccacagaaactcctcaaagtgcaagtccaagccccagatcgccgccctgaaggaggagaccgaggaggaggtgcaggacaccaggctgtga

SEQ ID NO: 29 - DNA, 2100 bp, Artificial Sequence
  Homo sapiens DNAI1 sequence, codon optimized, DNAI1 #1
atgatcccagcttctgccaaggccccacacaagcagccacacaaacagagcatttccattgggcgcggcacaaggaagagagacgaggactcaggcacagaggtgggcgaaggaaccgacgagtgggctcagagcaaagccacagtgaggcccccagatcagctggagctgacagacgccgagctgaaggaggagtttacccgcatcctgactgccaataacccacacgcaccccagaacatcgtgcgctattcttttaaggaaggaacctataagccaatcggctttgtcaatcagctggctgtgcactacacccaggttgggaacctgatccccaaggatagcgacgagggcaggagacagcattatagagacgagctcgtcgccggaagccaggagtctgtcaaagtgatcagcgaaacaggaaacctggaggaggatgaggagcccaaggaactggaaaccgagcctggcagccagacagatgtgccagccgcaggagccgcagagaaggtgacagaagaggagctcatgacccccaaacagccaaaggagcggaaactgacaaaccagttcaacttcagcgaaagagccagccagacctacaataaccccgtgcgggacagagaatgccagacagagcctccaccacgcaccaacttctccgcaacagctaaccagtgggagatctatgatgcctacgtggaggagctggaaaagcaggagaagaccaaagaaaaggagaaagccaagacccctgtcgccaagaagtccggcaaaatggctatgagaaagctgacatctatggaatcccagactgatgacctgatcaagctgtctcaggcagccaagattatggaaagaatggtgaatcagaacacctatgacgacatcgcccaggattttaagtactatgatgacgctgcagacgagtatagagatcaggtggggaccctgctgccactgtggaagttccagaatgacaaggctaagcgcctgtccgtgacagctctgtgctggaatccaaaatatagggacctcttcgccgtgggctacggctcttatgacttcatgaagcagtcacgcgggatgctgctgctgtacagcctgaaaaatccctcctttcccgagtacatgttcagctctaactccggggtcatgtgtctggatattcatgtggaccatccatacctggtggctgtcgggcactacgatggaaacgtggctatctacaatctgaagaagccacactcccagccctccttttgctcctccgccaagtccggcaagcactccgaccctgtgtggcaggtcaagtggcagaaggacgacatggaccagaacctgaacttcttttctgtgtctagcgatggcaggatcgtgtcctggaccctggtgaagagaaaactggtgcacatcgatgttatcaagctcaaagtcgagggaagcaccaccgaggttcctgagggcctgcagctgcacccagtgggctgcggcacagccttcgactttcataaagagattgactacatgttcctggtgggcacagaggaggggaagatctacaagtgctccaaatcctactccagccagtttctggacacttacgacgctcataatatgagcgtggacaccgtgtcctggaacccttaccacacaaaggtgttcatgagctgcagcagcgactggactgtgaagatttgggaccatactatcaaaaccccaatgtttatctatgatctcaattctgccgtgggcgacgtggcttgggccccctattcctccacagtgttcgcagccgtgactaccgacggaaaagcccacattttcgacctcgctattaacaagtatgaggccatttgtaaccagccagtggctgccaagaagaaccgcctgacccacgtgcagttcaacctgattcacccaattatcattgtgggggacgacagaggacacattatctcactgaagctgtctcctaatctgagaaagatgcctaaggagaagaaaggacaggaggtgcagaagggccctgccgtggaaattgccaaactcgacaagctgctgaacctggtgagggaggtgaagatcaagacatga

SEQ ID NO: 30 - DNA, 2100 bp, Artificial Sequence
  Homo sapiens DNAI1 sequence, codon optimized, DNAI1 #2
atgatccccgcatccgccaaagcccctcataaacagccccacaaacagtccatctccattggacgggggacccggaaaagggatgaggactctgggacggaagttggagaaggcactgacgaatgggcacagagtaaggctaccgtgagacctcccgaccagctggagctcactgacgcagaactgaaggaggagtttactaggatcctgacagcaaataacccccacgccccacagaatatcgtcagatatagcttcaaagagggcacatacaagcctattgggttcgtgaaccagctggctgtgcattacacacaggtggggaaccttattcctaaagactctgatgaaggccgcagacagcattatagagatgaactggttgcaggatcccaagagtctgtgaaagtgattagcgagaccggcaacctggaagaagatgaggaaccaaaagaactggagacagagcctgggtctcagacagacgtgccagcagctggcgctgccgagaaagtgacagaggaggagctgatgacacctaaacagccaaaagagaggaagctgacaaaccaattcaatttttccgaacgggcatcacagacctacaacaacccagtgcgcgaccgggagtgtcaaaccgaacctcctcctagaacaaacttttctgctactgcaaatcagtgggagatctacgatgcctacgtggaggagctggagaagcaggaaaagactaaggagaaggagaaggcaaagacccccgtggccaaaaaatccggcaaaatggcaatgcggaagctgacttctatggaaagccagactgatgacctgatcaaactgtcccaggcagctaagattatggaaaggatggtcaatcagaatacatatgacgacattgctcaggactttaagtattatgatgatgccgctgacgagtatcgggaccaagtggggacactgctgccactgtggaagtttcaaaacgacaaggctaaaaggctgtccgtgacagcactctgctggaatcccaagtaccgggacctctttgccgtggggtacggatcttacgacttcatgaaacagtccagaggcatgctgctgctgtacagcttgaagaacccctcctttcccgagtacatgttcagctctaattctggagtgatgtgcctggacatccacgtggatcacccttacctcgtggccgttggacactatgacggcaatgtggccatctacaacctgaaaaaaccacactctcagccttccttttgtagctctgcaaagtccggaaagcattccgaccccgtgtggcaagtgaaatggcagaaagacgacatggaccagaatctgaacttcttctccgtctcttcagacggcagaatcgtctcatggactctggtcaaacggaagctggttcacatcgacgtgatcaaactcaaggtcgaaggatcgactactgaggtgccagaaggactgcagctgcacccagtgggatgtggaactgcatttgatttccataaagaaatcgactacatgtttctggtgggaactgaagaggggaagatctataagtgtagcaaatcctattctagccagtttctggatacatacgacgctcacaacatgtccgtggacactgtaagctggaacccctatcataccaaggtgttcatgtcctgcagctccgattggactgttaagatttgggatcacacaatcaagacccctatgtttatctacgatctgaactctgccgtgggggatgtggcctgggcaccatatagctccacagtcttcgcagctgtcactaccgatggaaaggcccacatttttgacctggctatcaacaaatacgaggccatctgcaatcagcctgtggcagcaaagaagaaccgcctgactcacgtgcaattcaacctgattcaccctatcatcattgttggggatgataggggccacattatttctctaaagctgtccccaaatctgcggaaaatgcccaaggagaagaaaggccaggaggtgcagaaaggcccagccgttgaaatcgcaaagctggacaagctgctcaacctcgtccgggaggttaaaatcaaaacctga

SEQ ID NO: 31 - DNA, 2100 bp, Artificial Sequence
  Homo sapiens DNAI1 sequence, codon optimized, DNAI1 #3
atgatcccagcaagcgccaaggccccacacaaacagccccacaagcagtcgatcagcattggcagggggactcgcaagagagacgaggactccggaacagaagtgggggaggggacagatgaatgggcccagtctaaggccactgttcgccctccggatcagctggaactgacagatgccgagctgaaggaagagttcaccaggattctgactgcaaataatccacacgctccacagaacattgtgagatattcttttaaggagggcacttacaaacccatcgggtttgtgaatcagctggcagtgcattacactcaagtgggcaacctgatccccaaagactctgatgaagggaggcggcagcactatagggacgagctggtcgctgggtcccaagagagcgtgaaagtcatttctgagactggcaacctggaagaggatgaggagccaaaggagctggagactgaaccagggtctcagacagatgtgcccgccgctggagctgctgagaaggtgacagaggaggaactgatgacccctaaacagcctaaggaacggaagctcaccaaccagttcaacttcagcgaaagagctagccagacttataataaccctgtgcgcgaccgggagtgtcagactgagcccccaccaagaaccaatttctccgccactgccaaccagtgggaaatctatgacgcttacgtcgaggagctggagaaacaggagaaaactaaggagaaagaaaaggccaaaacacccgtcgccaaaaagtctggcaagatggccatgagaaaactgacctccatggagtctcagaccgacgacctgatcaaactgtcccaggcagccaagatcatggagaggatggtgaaccagaacacctatgatgacattgcccaggactttaaatactacgatgatgccgctgacgagtatcgggaccaggtggggactctgctgcctctgtggaaattccagaatgataaggctaaacgcctgtccgtgaccgccctctgctggaaccctaagtaccgcgacctctttgctgtggggtacggatcttacgacttcatgaaacagtccagaggcatgctgctgctgtacagcttgaagaacccctcctttcccgagtacatgttcagctctaattctggagtgatgtgcctggacatccacgtggatcacccttacctcgtggccgttggacactatgacggcaatgtggccatctacaacctgaaaaaaccacactctcagccttccttttgtagctctgcaaagtccggaaagcattccgaccccgtgtggcaagtgaaatggcagaaagacgacatggaccagaatctgaacttcttctccgtctcttcagacggcagaatcgtctcatggactctggtcaaacggaagctggttcacatcgacgtgatcaaactcaaggtcgaaggatcgactactgaggtgccagaaggactgcagctgcacccagtgggatgtggaactgcatttgatttccataaagaaatcgactacatgtttctggtgggaactgaagaggggaagatctataagtgtagcaaatcctattctagccagtttctggatacatacgacgctcacaacatgtccgtggacactgtaagctggaacccctatcataccaaggtgttcatgtcctgcagctccgattggactgttaagatttgggatcacacaatcaagacccctatgtttatctacgatctgaactctgccgtgggggatgtggcctgggcaccatatagctccacagtcttcgcagctgtcactaccgatggaaaggcccacatttttgacctggctatcaacaaatacgaggccatctgcaatcagcctgtggcagcaaagaagaaccgcctgactcacgtgcaattcaacctgattcaccctatcatcattgttggggatgataggggccacattatttctctaaagctgtccccaaatctgcggaaaatgcccaaggagaagaaaggccaggaggtgcagaaaggcccagccgttgaaatcgcaaagctggacaagctgctcaacctcgtccgggaggttaaaatcaaaacctga

SEQ ID NO: 32 - DNA, 2100 bp, Artificial Sequence
  Homo sapiens DNAI1 sequence, codon optimized, DNAI1 #4
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagacttaa